SEQUENCE LISTING (332 sequences)

SEQ ID NO: 1-299 - skipped (299 entries)

SEQ ID NO: 300 - DNA, 167 bp, Artificial Sequence
  Description of Artificial Sequence: Synthetic polynucleotide
aggaacccctagtgatggagttggccactccctctctgcgcgctcgctcgctcactgaggcaatttgataaaaatcgtcaaattataaacaggctttgcctgtttagcctcagtgagcgagcgagcgcgcagagagggagtggccaactccatcactaggggttcct

SEQ ID NO: 301 - DNA, 143 bp, Artificial Sequence
  Description of Artificial Sequence: Synthetic polynucleotide
aggaacccctagtgatggagttggccactccctctctgcgcgctcgctcgctcactgagggataaaaatccaggctttgcctgcctcagtgagcgagcgagcgcgcagagagggagtggccaactccatcactaggggttcct

SEQ ID NO: 302 - DNA, 143 bp, Artificial Sequence; identical to SEQ ID NO: 301
  Description of Artificial Sequence: Synthetic polynucleotide

SEQ ID NO: 303 - DNA, 208 bp, Artificial Sequence
  Description of Artificial Sequence: Synthetic polynucleotide
aggaacccctagtgatggagttggccactccctctctgggattgggattgcgcgctcgctcgcgggattgggattgggattgggattgggattgggattgataaaaatcaatcccaatcccaatcccaatcccaatcccaatcccgcgagcgagcgcgcaatcccaatcccagagagggagtggccaactccatcactaggggttcct

SEQ ID NO: 304 - DNA, 199 bp, Artificial Sequence
  Description of Artificial Sequence: Synthetic polynucleotide
aggaacccctagtgatggagttggccactccctctctgcgcgctcgctcgctcgggattgggattgggattgggattgggattgggattgataaaaatcaatcccaatcccaatcccaatcccaatcccaatcccgcgagcgagcgcgcaggagagggagtggccaactccatcactaggggttcctaagcttattata

SEQ ID NO: 305 - DNA, 154 bp, Artificial Sequence
  Description of Artificial Sequence: Synthetic polynucleotide
aggaacccctagtgatggagttggccactccctctctgcgcgctcgctcgctcactgagggcgcctataaagataaaaatccaggctttgcctgcctcagttagcgagcgagcgcgcagagagggagtggccaactccatcactaggggttcct

SEQ ID NO: 306 - DNA, 127 bp, Artificial Sequence
  Description of Artificial Sequence: Synthetic polynucleotide
ctagtgatggagttggccactccctctctgcgcgctcgctcgctcactgagggataaaaatccaggctttgcctgcctcagtgagcgagcgagcgcgcagagagggagtggccaactccatcactag

SEQ ID NO: 307 - protein, 1480 aa, Homo sapiens
MQRSPLEKASVVSKLFFSWTRPILRKGYRQRLELSDIYQIPSVDSADNLSEKLEREWDRELASKKNPKLINALRRCFFWRFMFYGIFLYLGEVTKAVQPLLLGRIIASYDPDNKEERSIAIYLGIGLCLLFIVRTLLLHPAIFGLHHIGMQMRIAMFSLIYKKTLKLSSRVLDKISIGQLVSLLSNNLNKFDEGLALAHFVWIAPLQVALLMGLIWELLQASAFCGLGFLIVLALFQAGLGRMMMKYRDQRAGKISERLVITSEMIENIQSVKAYCWEEAMEKMIENLRQTELKLTRKAAYVRYFNSSAFFFSGFFVVFLSVLPYALIKGIILRKIFTTISFCIVLRMAVTRQFPWAVQTWYDSLGAINKIQDFLQKQEYKTLEYNLTTTEVVMENVTAFWEEGFGELFEKAKQNNNNRKTSNGDDSLFFSNFSLLGTPVLKDINFKIERGQLLAVAGSTGAGKTSLLMVIMGELEPSEGKIKHSGRISFCSQFSWIMPGTIKENIIFGVSYDEYRYRSVIKACQLEEDISKFAEKDNIVLGEGGITLSGGQRARISLARAVYKDADLYLLDSPFGYLDVLTEKEIFESCVCKLMANKTRILVTSKMEHLKKADKILILHEGSSYFYGTFSELQNLQPDFSSKLMGCDSFDQFSAERRNSILTETLHRFSLEGDAPVSWTETKKQSFKQTGEFGEKRKNSILNPINSIRKFSIVQKTPLQMNGIEEDSDEPLERRLSLVPDSEQGEAILPRISVISTGPTLQARRRQSVLNLMTHSVNQGQNIHRKTTASTRKVSLAPQANLTELDIYSRRLSQETGLEISEEINEEDLKECFFDDMESIPAVTTWNTYLRYITVHKSLIFVLIWCLVIFLAEVAASLVVLWLLGNTPLQDKGNSTHSRNNSYAVIITSTSSYYVFYIYVGVADTLLAMGFFRGLPLVHTLITVSKILHHKMLHSVLQAPMSTLNTLKAGGILNRFSKDIAILDDLLPLTIFDFIQLLLIVIGAIAVVAVLQPYIFVATVPVIVAFIMLRAYFLQTSQQLKQLESEGRSPIFTHLVTSLKGLWTLRAFGRQPYFETLFHKALNLHTANWFLYLSTLRWFQMRIEMIFVIFFIAVTFISILTTGEGEGRVGIILTLAMNIMSTLQWAVNSSIDVDSLMRSVSRVFKFIDMPTEGKPTKSTKPYKNGQLSKVMIIENSHVKKDDIWPSGGQMTVKDLTAKYTEGGNAILENISFSISPGQRVGLLGRTGSGKSTLLSAFLRLLNTEGEIQIDGVSWDSITLQQWRKAFGVIPQKVFIFSGTFRKNLDPYEQWSDQEIWKVADEVGLRSVIEQFPGKLDFVLVDGGCVLSHGHKQLMCLARSVLSKAKILLLDEPSAHLDPVTYQIIRRTLKQAFADCTVILCEHRIEAMLECQQFLVIEENKVRQYDSIQKLLNERSLFRQAISPSDRVKLFPHRNSSKCKSKPQIAALKEETEEEVQDTRL

SEQ ID NO: 308 - DNA, 23 bp, Artificial Sequence
  Description of Artificial Sequence: Synthetic oligonucleotide
  modified_base (1)..(20): a, c, t, or g
  modified_base (21)..(21): a, c, t, g, unknown or other
nnnnnnnnnnnnnnnnnnnnngg

SEQ ID NO: 309 - DNA, 15 bp, Artificial Sequence
  Description of Artificial Sequence: Synthetic oligonucleotide
  modified_base (1)..(12): a, c, t, or g
  modified_base (13)..(13): a, c, t, g, unknown or other
nnnnnnnnnnnnngg

SEQ ID NO: 310 - DNA, 23 bp, Artificial Sequence
  Description of Artificial Sequence: Synthetic oligonucleotide
  modified_base (1)..(20): a, c, t, or g
  modified_base (21)..(21): a, c, t, g, unknown or other
nnnnnnnnnnnnnnnnnnnnngg

SEQ ID NO: 311 - DNA, 14 bp, Artificial Sequence
  Description of Artificial Sequence: Synthetic oligonucleotide
  modified_base (1)..(11): a, c, t, or g
  modified_base (12)..(12): a, c, t, g, unknown or other
nnnnnnnnnnnngg

SEQ ID NO: 312 - DNA, 27 bp, Artificial Sequence
  Description of Artificial Sequence: Synthetic oligonucleotide
  modified_base (1)..(20): a, c, t, or g
  modified_base (21)..(22): a, c, t, g, unknown or other
nnnnnnnnnnnnnnnnnnnnnnagaaw

SEQ ID NO: 313 - DNA, 19 bp, Artificial Sequence
  Description of Artificial Sequence: Synthetic oligonucleotide
  modified_base (1)..(12): a, c, t, or g
  modified_base (13)..(14): a, c, t, g, unknown or other
nnnnnnnnnnnnnnagaaw

SEQ ID NO: 314 - DNA, 27 bp, Artificial Sequence
  Description of Artificial Sequence: Synthetic oligonucleotide
  modified_base (1)..(20): a, c, t, or g
  modified_base (21)..(22): a, c, t, g, unknown or other
nnnnnnnnnnnnnnnnnnnnnnagaaw

SEQ ID NO: 315 - DNA, 18 bp, Artificial Sequence
  Description of Artificial Sequence: Synthetic oligonucleotide
  modified_base (1)..(11): a, c, t, or g
  modified_base (12)..(13): a, c, t, g, unknown or other
nnnnnnnnnnnnnagaaw

SEQ ID NO: 316 - DNA, 25 bp, Artificial Sequence
  Description of Artificial Sequence: Synthetic oligonucleotide
  modified_base (1)..(20): a, c, t, or g
  modified_base (21)..(21): a, c, t, g, unknown or other
  modified_base (24)..(24): a, c, t, g, unknown or other
nnnnnnnnnnnnnnnnnnnnnggng

SEQ ID NO: 317 - DNA, 17 bp, Artificial Sequence
  Description of Artificial Sequence: Synthetic oligonucleotide
  modified_base (1)..(12): a, c, t, or g
  modified_base (13)..(13): a, c, t, g, unknown or other
  modified_base (16)..(16): a, c, t, g, unknown or other
nnnnnnnnnnnnnggng

SEQ ID NO: 318 - DNA, 25 bp, Artificial Sequence
  Description of Artificial Sequence: Synthetic oligonucleotide
  modified_base (1)..(20): a, c, t, or g
  modified_base (21)..(21): a, c, t, g, unknown or other
  modified_base (24)..(24): a, c, t, g, unknown or other
nnnnnnnnnnnnnnnnnnnnnggng

SEQ ID NO: 319 - DNA, 16 bp, Artificial Sequence
  Description of Artificial Sequence: Synthetic oligonucleotide
  modified_base (1)..(11): a, c, t, or g
  modified_base (12)..(12): a, c, t, g, unknown or other
  modified_base (15)..(15): a, c, t, g, unknown or other
nnnnnnnnnnnnggng

SEQ ID NO: 320 - DNA, 137 bp, Artificial Sequence
  Description of Artificial Sequence: Synthetic polynucleotide
  modified_base (1)..(20): a, c, t, g, unknown or other
nnnnnnnnnnnnnnnnnnnngtttttgtactctcaagatttagaaataaatcttgcagaagctacaaagataaggcttcatgccgaaatcaacaccctgtcattttatggcagggtgttttcgttatttaatttttt

SEQ ID NO: 321 - DNA, 123 bp, Artificial Sequence
  Description of Artificial Sequence: Synthetic polynucleotide
  modified_base (1)..(20): a, c, t, g, unknown or other
nnnnnnnnnnnnnnnnnnnngtttttgtactctcagaaathcagaagctacaaagataaggcttcatgccgaaatcaacaccctgtcattttatggcagggtgttttcgttatttaatttttt

SEQ ID NO: 322 - DNA, 110 bp, Artificial Sequence
  Description of Artificial Sequence: Synthetic polynucleotide
  modified_base (1)..(20): a, c, t, g, unknown or other
nnnnnnnnnnnnnnnnnnnngtttttgtactctcagaaatgcagaagctacaaagataaggcttcatgccgaaatcaacaccctgtcattttatggcagggtgttttttt

SEQ ID NO: 323 - DNA, 102 bp, Artificial Sequence
  Description of Artificial Sequence: Synthetic polynucleotide
  modified_base (1)..(20): a, c, t, g, unknown or other
nnnnnnnnnnnnnnnnnnnngttttagagctagaaatagcaagttaaaataaggctagtccgttatcaacttgaaaaagtggcaccgagtcggtgctttttt

SEQ ID NO: 324 - DNA, 87 bp, Artificial Sequence
  Description of Artificial Sequence: Synthetic oligonucleotide
  modified_base (1)..(20): a, c, t, g, unknown or other
nnnnnnnnnnnnnnnnnnnngttttagagctagaaatagcaagttaaaataaggctagtccgttatcaacttgaaaaagtttttttt

SEQ ID NO: 325 - DNA, 76 bp, Artificial Sequence
  Description of Artificial Sequence: Synthetic oligonucleotide
  modified_base (1)..(20): a, c, t, g, unknown or other
nnnnnnnnnnnnnnnnnnnngttttagagctagaaatagcaagttaaaataaggctagtccgttatcatttttttt

SEQ ID NO: 326 - DNA, 20 bp, Homo sapiens
cgctctatcgcgatttatct

SEQ ID NO: 327 - DNA, 20 bp, Homo sapiens
gagcgttcctccttgttatc

SEQ ID NO: 328 - DNA, 20 bp, Homo sapiens
tccagaaaaaacatcgccga

SEQ ID NO: 329 - DNA, 20 bp, Homo sapiens
ggtatatgtctgacaattcc

SEQ ID NO: 330 - DNA, 20 bp, Artificial Sequence
  Description of Artificial Sequence: Synthetic oligonucleotide
cgctagagcaaatttggggc

SEQ ID NO: 331 - DNA, 20 bp, Artificial Sequence
  Description of Artificial Sequence: Synthetic oligonucleotide
gggcggcgagggagcgaagg

SEQ ID NO: 332 - DNA, 20 bp, Artificial Sequence
  Description of Artificial Sequence: Synthetic oligonucleotide
tggcgggggtgcgtagtggg

(19) United States
(12) Patent Application Publication
(10) Pub. No.: US 2022/0152221 A1
(43) Pub. Date: May 19, 2022

(54) A METHOD OF TREATING CYSTIC FIBROSIS

(71) Applicant: ASKLEPIOS BIOPHARMACEUTICAL, INC., Research Triangle Park, NC (US)

(72) Inventor: Michael W. O'CALLAGHAN, Research Triangle Park, NC (US)

(73) Assignee: ASKLEPIOS BIOPHARMACEUTICAL, INC., Research Triangle Park, NC (US)

(21) Appl. No.: 17/421,277

(22) PCT Filed: Jan. 7, 2020
(86) PCT No.: PCT/US2020/012574

§ 371 Date: Jul. 7, 2021

Related U.S. Application Data
(60) Provisional application No. 62/870,358, filed on Jul. 3, 2019.
(60) Provisional application No. 62/865,731, filed on Jun. 24, 2019.
(60) Provisional application No. 62/789,797, filed on Jan. 8, 2019.

(51) Int. Cl. A61K 48/00; C12N 15/86; A61P 11/00

(52) U.S. Cl. CPC A61K 48/005; A61P 11/00; C12N 15/86

(57) ABSTRACT

Described herein are methods and compositions related to vectors, including but not limited to a method for treating cystic fibrosis (CF) using adeno-associated vims (AAV) particles, using a catheter to administer a population of viral vectors to a plurality of target sites in a subject by bronchial artery catheterization delivery.

### CROSS-REFERENCE TO RELATED APPLICATIONS

This application claims benefit under 35 U.S.C. § 119(e) of U.S. Provisional Application Nos. 62/789,797 filed Jan. 8, 2019, 62/865,731 filed Jun. 24, 2019 and 62/870,358 filed Jul. 3, 2019 the content of each of which is incorporated herein by reference in its entirety.

### TECHNICAL FIELD

The present invention relates to using bronchial artery delivery to administer therapeutic vectors to the lungs, including but not limited to adeno-associated virus (AAV) particles, virions and vectors for the treatment of cystic fibrosis.

### BACKGROUND

Gene therapy has been shown to have the potential to not only cure genetic disorders, but to also facilitate the long-term non-invasive treatment of acquired and degenerative disease using a virus, such as an adeno-associated virus (AAV). AAV itself is a non-pathogenic-dependent parvovirus that needs helper viruses for efficient replication. AAV has been utilized as a virus vector for gene therapy because of its safety and simplicity. AAV has a broad host and cell type tropism capable of transducing both dividing and non-dividing cells. To date, 12 AAV serotypes and more than 100 variants have been identified. It has been shown that the different AAV serotypes can have differing abilities to infect cells of different tissues, either in vivo or in vitro and that these differences in infectivity are likely tied to the particular receptors and co-receptors located on the capsid surface of each AAV serotype or may be tied to the intracellular trafficking pathway itself.

Accordingly, as an alternative or adjunct to enzyme therapy, the feasibility of gene therapy approaches to treat diseases e.g. hemophilia have been investigated (High K. A., et. al., (2016) Hum. Mol. Genet. April 15; 25(R1):R36-41; Samelson-Jones B. J., et. al. (2018) Mol Ther Methods Clin Dev. 2018 Dec. 31; 12:184-201).

Cystic fibrosis (CF) is a disease characterized by airway infection, inflammation, remodeling, and obstruction that gradually destroy the lungs and is the most common fatal hereditary lung disease. CF is an autosomal recessive disorder characterized by abnormalities in water and electrolyte transport that lead to pancreatic and pulmonary insufficiency. It is one of the most common severe autosomal recessive disorders, having a 5% carrier frequency and affecting about 1 in 2500 live births in North America.

CF is a recessive disease caused by mutations in the cystic fibrosis transmembrane conductance regulator (CFTR) gene, which encodes an anion channel regulated by ATP hydrolysis and phosphorylation. CF is an attractive candidate for gene therapy because heterozygotes are phenotypically normal and the target cells lining the intrapulmonary airways are potentially accessible for vector delivery via aerosol, topical strategies, or vascular strategies.

There is no known cure for cystic fibrosis. The average life expectancy is between 42 and 50 years in the developed world. Lung problems are responsible for death in 80% of people with cystic fibrosis.

The following CF disease-specific therapies include KALYDECO® (ivacaftor) tablets for oral use. Initial U.S. Approval: 2012 directed to milder (and rarer) mutations that still produce CFTR protein on the epithelial cell surface, ORKAMBI® (lumacaftor/ivacaftor) tablets for oral use. U.S. Approval: 2015 for treatment of CF patients with two copies of the F508del mutation (F508del/F508del) directed to for the most common severe mutation, and SYMDEKO™ (tezacaftor/ivacaftor) tablets for oral use. Initial U.S. Approval: 2018 directed to treatment of single F508del heterozygotes and some other mutations not covered by Kalydeco

Symptomatic treatments include nebulized hypertonic saline, dornase alfa and mannitol dry powder to reduce viscosity of airway mucus; antibiotics (often nebulized) to treat endemic Pseudomonas aeruginosa infections; bronchodilators to improve airway patency, steroids, daily chest massage, vibration and pounding to loosen secretions.

Thus there is significant unmet medical need, particularly for the most common, severe mutations. Delivery of therapeutics to the target cell population of CF remains a major challenge. Therefore, there is a need in the art for methods for the treatment of CF using safe and efficient vector systems approaches targeting the basic ion transport defect in CF airways by delivery of the wildtype CFTR gene to the lung tissue.

### SUMMARY OF THE INVENTION

The technology described herein relates generally to a gene therapy approach using bronchial artery delivery to administer vectors, including but not limited to adeno-associated virus (AAV) particles, virions and vectors for the treatment of CF.

Accordingly, described herein are catheters being used to administer viral vectors, e.g., using rAAV vectors as an exemplary example, that comprises a nucleotide sequence containing inverted terminal repeats (ITRs), a promoter, a heterologous gene, a poly-A tail and potentially other regulator elements for use to treat cystic fibrosis.

CF is a disease characterized by airway infection, inflammation, remodeling, and obstruction that gradually destroy the lungs. Physical and host immune barriers in the lung present challenges for successful gene transfer to the respiratory tract. CF is inherited in an autosomal recessive manner. It is caused by the presence of mutations in both copies of the gene for the cystic fibrosis transmembrane conductance regulator (CFTR) protein. CFTR is a membrane protein and chloride channel in vertebrates that is encoded by the CFTR gene. Those with a single working copy of CFTR are carriers and otherwise mostly normal. CFTR is involved in production of sweat, digestive fluids, and mucus. When the CFTR is not functional, secretions which are usually thin and fluid instead become thick and viscous. The condition is diagnosed by a sweat test and genetic testing. Screening of infants at birth takes place in some areas of the world.

The CFTR gene is an attractive candidate for gene therapy because heterozygotes are phenotypically normal and the target cells lining the intrapulmonary airways are potentially accessible for vector delivery via aerosol or other topical strategies. Since the CFTR gene was first cloned in 1989, several gene therapy strategies for correction of CF lung disease have been investigated. However, the development of safe and efficient vector systems remains a major challenge. This is due, in part, to the multiple, sophisticated pulmonary barriers that have evolved to clear or prevent the uptake of foreign particles. Thick secretions and the secondary effects of chronic infection and inflammation in the CF lung present additional barriers to gene transfer.

As described herein, is a method for treating CF by direct delivery of the cystic fibrosis transmembrane conductance regulator (CFTR) gene to the lungs. Aspects of the present invention teach certain benefits in construction and use which give rise to the exemplary advantages described below.

In some embodiments, disclosed herein is a pharmaceutical formulation comprising a targeting viral vector, e.g the therapeutic construct can comprise (1) any of the 12 naturally occurring AAV capsids, any of the engineered variants thereof, or any related dependoviruses such as avian or canine AAV, (2) the cDNA transgene of CFTR or variants thereof, (3) promoter and enhancer elements tailored for best expression and (4) a pharmaceutically acceptable carrier or excipient.

Also, in some embodiments, relates to use of a viral vector, e.g., rAAV vectors, nucleic acid encoding a viral vector genome as disclosed herein, in the treatment of cystic fibrosis.

Aspects of the technology described herein are outlined here, wherein the viral vector comprises, in the 5′ to 3′ direction:

### a 5′ ITR,

a promoter sequence,
an intron sequence,
a therapeutic transgene (e.g. the wild-type CFTR gene),
a poly A sequence, and

### a 3′ ITR.

Accordingly, provided herein, in some aspects, a method for treating cystic fibrosis (CF) comprising: administering a population of vectors to a plurality of target sites in a subject wherein the vector contains a therapeutic nucleic acid, and wherein the vectors are administered by bronchial artery catheterization delivery comprising, placing a catheter into a first bronchial artery and administering a first dose of vector into the catheter to target the first basal lamina target sites in a first family of bronchioles, and placing the same or different catheter into a second bronchial artery to target a second set of basal laminar cells in the family of bronchioles subtending the second bronchial artery. As necessary a third or even fourth injection into a third or fourth variant brochial arteries to complete therapeutic delivery to all basal laminar cells.

In some embodiments of these methods and all such methods described herein, the first dose is proportional to the first bronchial artery volume (the bronchial vessel blood flow volume including the vessel branches) and the second, third or fourth dose is proportional to the total bronchial artery volume. In some embodiments of these methods and all such methods described herein, the first dose of vector is administered into the catheter to target basal lamina target sites of basal/progenitor cells, club cells, or ciliated cells in all of the bronchioles subtended by delivery to the first bronchial artery.

In some embodiments of these methods and all such methods described herein, the therapeutic nucleic acid is a therapeutic Cystic Fibrosis Transmembrane Conductance Regulator (CFTR) gene.

In some embodiments of these methods and all such methods described herein, the therapeutic nucleic acid is a truncated therapeutic Cystic Fibrosis Transmembrane Conductance Regulator (CFTR) gene.

In some embodiments of these methods and all such methods described herein, the truncated therapeutic Cystic Fibrosis Transmembrane Conductance Regulator (CFTR) gene is a N-tail processing mutants of CFTR.

In some embodiments of these methods and all such methods described herein, the truncated therapeutic Cystic Fibrosis Transmembrane Conductance Regulator (CFTR) gene can specifically rescue the processing of ΔF508-CFTR.

In some embodiments of these methods and all such methods described herein, the vector is a DNA or RNA nucleic acid vector.

In some embodiments of these methods and all such methods described herein, vector is a viral vector.

In some embodiments of these methods and all such methods described herein, viral vector is selected from any of: an adeno associated virus (AAV), adenovirus, lentivirus vector, or a herpes simplex virus (HSV).

In some embodiments of these methods and all such methods described herein, the viral vector is a recombinant AAV (rAAV).

In some embodiments of these methods and all such methods described herein, the therapeutic nucleic acid is a gene editing molecule.

In some embodiments of these methods and all such methods described herein, gene editing molecule is selected from a nuclease, a guide RNA (gRNA), a guide DNA (gDNA), and an activator RNA.

In some embodiments of these methods and all such methods described herein, at least one gene editing molecule is a gRNA or a gDNA.

In some embodiments of these methods and all such methods described herein, the guide RNA is targeting a pathology-causing CFTR gene.

In some embodiments of these methods and all such methods described herein, the guide RNA is selected from Table 4.

In some embodiments of these methods and all such methods described herein, the sequence specific nuclease is selected from a nucleic acid-guided nuclease, zinc finger nuclease (ZFN), a meganuclease, a transcription activator-like effector nuclease (TALEN), or a megaTAL.

In some embodiments of these methods and all such methods described herein, the sequence specific nuclease is a nucleic acid-guided nuclease selected from a single-base editor, an RNA-guided nuclease, and a DNA-guided nuclease.

In some embodiments of these methods and all such methods described herein, at least one gene editing molecule is an activator RNA.

In some embodiments of these methods and all such methods described herein, the nucleic acid-guided nuclease is a CRISPR nuclease.

In some embodiments of these methods and all such methods described herein, the CRISPR nuclease is a Cas nuclease.

In some embodiments of these methods and all such methods described herein, the bronchial artery delivery is accompanied by a separate pulmonary artery catheterization to obtain a a wedge pressure measurement.

In some embodiments of these methods and all such methods described herein, the population of viral vectors is administered by slow infusion over one to five minutes.

In some embodiments of these methods and all such methods described herein, pressure is applied to expiratory airflow either in periodic intervals or pulsed intervals during infusion.

In some embodiments of these methods and all such methods described herein, the pressure is supplied every second to fifth breath for up to 15 seconds.

In some embodiments of these methods and all such methods described herein, the pressure is 2-15 mmHg.

In some embodiments of these methods and all such methods described herein, the proximity of bronchial artery capillaries carrying the vector to the target cells is 5 to 10 microns.

In some embodiments of these methods and all such methods described herein, the AAV of the capsid proteins and ITR can be any natural or artificial serotype or modifications thereof. The proteins and ITRs can be the same or different serotypes. In one embodiment, at least one of the AAV of the capsid protein is AAV serotype 9.

In another embodiment of any of the aspects, all capsid proteins are from AAV9.

In some embodiments of these methods and all such methods described herein, further comprising administration of a permeabilization agent.

In some embodiments of any of the aspects, at least one of the capsid proteins is AAV serotype 9.

In some embodiments of any of the aspects, all the capsid proteins are AAV serotype 9.

In some embodiments of any of the aspects, one of the other capsid proteins is from a different serotype.

In some embodiments of any of the aspects, the AAV ITRs are from different serotypes than at least one capsid protein.

In some embodiments of any of the aspects, the AAV ITRs are from at least one of the same serotypes as the capsid proteins.

Other features and advantages of aspects of the present invention will become apparent from the following more detailed description, taken in conjunction with the accompanying drawings, which illustrate, by way of example, the principles of aspects of the invention.

### DETAILED DESCRIPTION

Described herein is a method for treating cystic fibrosis (CF) using a catheter to administer a population of viral vectors, wherein the viral vector contains a therapeutic transgene to a plurality of target sites in a subject by bronchial artery catheterization delivery, placing the catheter proximally in the first bronchial artery, wherein the target site is basal/progenitor cells in the family of brochioles subtended by said bronchial artery, then moving the catheter into a second bronchial artery to deliver a second dose of viral vectors to a second population of basal/progenitor cells in the second family of brochioles subtended by the second bronchial artery. As necessitated by individual anatomy a third or fourth injection into a third or fourth bronchial artery or branch thereof would complete vector delivery.

One aspect of the technology described herein relates to a rAAV vector that comprises a nucleotide sequence containing inverted terminal repeats (ITRs), a promoter, a heterologous gene, a poly-A tail and potentially other regulator elements for use to treat cystic fibrosis. The nucleic acid is typically encapsulated in an AAV capsid. In some embodiments, the capsid can be a modified capsid. The capsid proteins can be from any AAV serotypes different from either ITR. The technology described herein relates generally to a gene therapy approach using bronchial artery delivery to administer vectors, including but not limited to adeno-associated virus (AAV) particles, virions and vectors for the treatment of CF.

Accordingly, described herein are catheters being used to administer viral vectors, e.g., using rAAV vectors as an exemplary example, that comprises a nucleotide sequence containing inverted terminal repeats (ITRs), a promoter, a heterologous gene, a poly-A tail and potentially other regulator elements for use to treat cystic fibrosis.

CF is a disease characterized by airway infection, inflammation, remodeling, and obstruction that gradually destroy the lungs. Physical and host immune barriers in the lung present challenges for successful gene transfer to the respiratory tract. CF is inherited in an autosomal recessive manner. It is caused by the presence of mutations in both copies of the gene for the cystic fibrosis transmembrane conductance regulator (CFTR) protein. Cystic fibrosis transmembrane conductance regulator (CFTR) is a membrane protein and chloride channel in vertebrates that is encoded by the CFTR gene. Those with a single working copy of CFTR are carriers and otherwise mostly normal. CFTR is involved in production of sweat, digestive fluids, and mucus. When the CFTR is not functional, secretions which are usually thin instead become thick. The condition is diagnosed by a sweat test and genetic testing. Screening of infants at birth takes place in some areas of the world.

The CFTR gene is an attractive candidate for gene therapy because heterozygotes are phenotypically normal and the target cells lining the intrapulmonary airways are potentially accessible for vector delivery via aerosol or other topical strategies. Since the CFTR gene was first cloned in 1989, several gene therapy strategies for correction of CF lung disease have been investigated. However, the development of safe and efficient vector systems remains a major challenge. This is due, in part, to the multiple, sophisticated pulmonary airway barriers that have evolved to clear or prevent the uptake of foreign particles. Thick secretions and the secondary effects of chronic infection and inflammation in the CF lung present additional barriers to gene transfer.

As described herein, is a method for treating CF by direct delivery of the cystic fibrosis transmembrane conductance regulator (CFTR) gene to the lungs. Aspects of the present invention teach certain benefits in construction and use which give rise to the exemplary advantages described below.

In some embodiments, disclosed herein is a pharmaceutical formulation comprising a targeting viral vector, e.g., rAAV vectors, nucleic acid encoding a rAAV as disclosed herein, and a pharmaceutically acceptable carrier. Also, in some embodiments, relates to use of a viral vector, e.g., rAAV vectors, nucleic acid encoding a viral vector genome as disclosed herein, in the treatment of cystic fibrosis.

Aspects of the technology described herein are outlined here, wherein the rAAV genome comprises, in the 5′ to 3′ direction: a 5′ ITR, a promoter sequence, an intron sequence, a therapeutic transgene (e.g. the wild-type CFTR gene), a poly A sequence, and a 3′ ITR.

In an embodiment, the rAAV vector comprises a viral capsid and within the capsid a cassette containing a nucleotide sequence, herein referred to as the “rAAV vector. The rAAV genome includes multiple elements, including, but not limited to two inverted terminal repeats (ITRs, e.g., the 5′-ITR and the 3′-ITR), and located between the ITRs are additional elements, including a promoter, a heterologous gene and a poly-A tail. In a further embodiment, there can be additional elements between the ITRs including seed region sequences for the binding of miRNA or an shRNA sequence. rAAV vectors for packaging do not include the enzymatic genes in the genome such as the rep proteins or the structural genes such as vp1, 2, or 3 because of size limitations. Capsids are typically prepared in trans. Similarly, the appropriate rep protein is expressed in trans.

### I. Definitions

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in immunology and molecular biology can be found in The Merck Manual of Diagnosis and Therapy, 20th Edition, published by Merck Sharp & Dohme Corp., 2018 (ISBN 0911910190, 978-0911910421); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by werner Luttmann, published by Elsevier, 2006; Janeway's Immunobiology, Kenneth Murphy, Allan Mowat, Casey weaver (eds.), W. W. Norton & Company, 2016 (ISBN 0815345054, 978-0815345053); Lewin's Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

The following terms are used in the description herein and the appended claims:

The terms “a,” “an,” “the” and similar references used in the context of describing the present invention (especially in the context of the following claims) are to be construed to cover both the singular and the plural, unless otherwise indicated herein or clearly contradicted by context. Further, ordinal indicators—such as “first,” “second,” “third,” etc.—for identified elements are used to distinguish between the elements, and do not indicate or imply a required or limited number of such elements, and do not indicate a particular position or order of such elements unless otherwise specifically stated. All methods described herein can be performed in any suitable order unless otherwise indicated herein or otherwise clearly contradicted by context. The use of any and all examples, or exemplary language (e.g., “such as”) provided herein is intended merely to better illuminate the present invention and does not pose a limitation on the scope of the invention otherwise claimed. No language in the present specification should be construed as indicating any non-claimed element essential to the practice of the invention.

Furthermore, the term “about,” as used herein when referring to a measurable value such as an amount of the length of a polynucleotide or polypeptide sequence, dose, time, temperature, and the like, is meant to encompass variations of ±20%, ±10%, ±5%, ±1%, ±0.5%, or even ±0.1% of the specified amount.

Also as used herein, “and/or” refers to and encompasses any and all possible combinations of one or more of the associated listed items, as well as the lack of combinations when interpreted in the alternative (“or”).

As used herein, the transitional phrase “consisting essentially of” means that the scope of a claim is to be interpreted to encompass the specified materials or steps recited in the claim, “and those that do not materially affect the basic and novel characteristic(s)” of the claimed invention. See, In re Herz, 537 F.2d 549, 551-52, 190 USPQ 461,463 (CCPA 1976) (emphasis in the original); see also MPEP § 2111.03. Thus, the term “consisting essentially of’ when used in a claim of this invention is not intended to be interpreted to be equivalent to “comprising.” Unless the context indicates otherwise, it is specifically intended that the various features of the invention described herein can be used in any combination.

Moreover, the present invention also contemplates that in some embodiments of the invention, any feature or combination of features set forth herein can be excluded or omitted.

To illustrate further, if, for example, the specification indicates that a particular amino acid can be selected from A, G, I, Land/or V, this language also indicates that the amino acid can be selected from any subset of these amino acid(s) for example A, G, I or L; A, G, I or V; A or G; only L; etc. as if each such subcombination is expressly set forth herein. Moreover, such language also indicates that one or more of the specified amino acids can be disclaimed (e.g., by negative proviso). For example, in particular embodiments the amino acid is not A, G or I; is not A; is not G or V; etc. as if each such possible disclaimer is expressly set forth herein.

The term “parvovirus” as used herein encompasses the family Parvoviridae, including autonomously replicating parvoviruses and dependoviruses. The autonomous parvoviruses include members of the genera Parvovirus, Erythrovirus, Densovirus, Iteravirus, and Contravirus. Exemplary autonomous parvoviruses include, but are not limited to, minute virus of mouse, bovine parvovirus, canine parvovirus, chicken parvovirus, feline panleukopenia virus, feline parvovirus, goose parvovirus, H1 parvovirus, Muscovy duck parvovirus, B19 virus, and any other autonomous parvovirus now known or later discovered. Other autonomous parvoviruses are known to those skilled in the art. See, e.g., BERNARD N. FIELDS et al., VIROLOGY, volume 2, chapter 69 (4th ed., Lippincott-Raven Publishers).

As used herein, the term “adeno-associated virus” (AAV), includes but is not limited to, AAV type 1, AAV type 2, AAV type 3 (including types 3A and 3B), AAV type 4, AAV type 5, AAV type 6, AAV type 7, AAV type 8, AAV type 9, AAV type 10, AAV type 11, AAV type 12, AAV type 13, avian AAV, bovine AAV, canine AAV, equine AAV, ovine AAV, and any other AAV now known or later discovered. See, e.g., BERNARD N. FIELDS et al., VIROLOGY, volume 2, chapter 69 (4th ed., Lippincott-Raven Publishers). A number of relatively new AAV serotypes and clades have been identified (see, e.g., Gao et al., (2004) J. Virology 78:6381-6388; Mons et al., (2004) Virology 33-:375-383). Chimeric, hybrid, mosaic, or rational haploids, which include mixtures of serotypes can also be used.

The genomic sequences of various serotypes of AAV and the autonomous parvoviruses, as well as the sequences of the native inverted terminal repeats (ITRs), Rep proteins, and capsid subunits are known in the art. Such sequences may be found in the literature or in public databases such as GenBank. See, e.g., GenBank Accession Numbers NC_002077, NC_001401, NC_001729, NC_001863, NC_001829, NC_001862, NC_000883, NC_001701, NC_001510, NC_006152, NC_006261, AF063497, U89790, AF043303, AF028705, AF028704, J02275, J01901, J02275, X01457, AF288061, AH009962, AY028226, AY028223, NC_001358, NC_001540, AF513851, AF513852, AY530579; the disclosures of which are incorporated by reference herein for teaching parvovirus and AAV nucleic acid and amino acid sequences. See also, e.g., Srivistava et al., (1983) J Virology 45:555; Chiarini et al., (1998) J. Virology 71:6823; Chiarini et al., (1999) J. Virology 73:1309; Bantel-Schaal et al., (1999) J. Virology 73:939; Xiao et al., (1999) J. Virology 73:3994; Muramatsu et al., (1996) Virology 221:208; Shade et al., (1986) J. Viral. 58:921; Gao et al., (2002) Proc. Nat. Acad. Sci. USA 99:11854; Morris et al., (2004) Virology 33-:375-383; international patent publications WO 00/28061, WO 99/61601, WO 98/11244; and U.S. Pat. No. 6,156,303; the disclosures of which are incorporated by reference herein for teaching parvovirus and AAV nucleic acid and amino acid sequences.

The capsid structures of autonomous parvoviruses and AAV are described in more detail in BERNARD N. FIELDS et al., VIROLOGY, volume 2, chapters 69 & 70 (4th ed., Lippincott-Raven Publishers). See also, description of the crystal structure of AAV2 (Xie et al., (2002) Proc. Nat. Acad. Sci. 99:10405-10), AAV4 (Padron et al., (2005) J. Viral. 79: 5047-58), AAV5 (Walters et al., (2004) J. Viral. 78: 3361-71) and CPV (Xie et al., (1996) J Mal. Biol. 6:497-520 and Tsao et al., (1991) Science 251: 1456-64).

The term “tropism” as used herein refers to preferential entry of the virus into certain cells or tissues, optionally followed by expression (e.g., transcription and, optionally, translation) of a sequence(s) carried by the viral genome in the cell, e.g., for a recombinant virus, expression of a heterologous nucleic acid(s) of interest.

As used here, “systemic tropism” and “systemic transduction” (and equivalent terms) indicate that the virus capsid or virus vector of the invention exhibits tropism for and/or transduces tissues throughout the body (e.g., brain, lung, skeletal muscle, heart, liver, kidney and/or pancreas). In embodiments of the invention, systemic transduction of the central nervous system (e.g., brain, neuronal cells, etc.) is observed. In other embodiments, systemic transduction of cardiac muscle tissues is achieved.

As used herein, “selective tropism” or “specific tropism” means delivery of virus vectors to and/or specific transduction of certain target cells and/or certain tissues.

In some embodiments of this invention, an AAV particle comprising a capsid of this invention can demonstrate multiple phenotypes of efficient transduction of 30 certain tissues/cells and very low levels of transduction (e.g., reduced transduction) for certain tissues/cells, the transduction of which is not desirable.

As used herein, the term “polypeptide” encompasses both peptides and proteins, unless indicated otherwise.

As used herein, the term “bronchial artery delivery” refers to insertion of a catheter into the bronchial arteries. Bronchial arteries are the sole vascular supply of the airways (and airways epithelium) down to the respiratory bronchioles.

A “polynucleotide” is a sequence of nucleotide bases, and may be RNA, DNA or DNA-RNA hybrid sequences (including both naturally occurring and non-naturally occurring nucleotides), but in representative embodiments are either single or double stranded DNA sequences.

A “chimeric nucleic acid” comprises two or more nucleic acid sequences covalently linked together to encode a fusion polypeptide. The nucleic acids may be DNA, RNA, or a hybrid thereof.

The term “fusion polypeptide” comprises two or more polypeptides covalently linked together, typically by peptide bonding.

As used herein, an “isolated” polynucleotide (e.g., an “isolated DNA” or an “isolated RNA”) means a polynucleotide at least partially separated from at least some of the other components of the naturally occurring organism or virus, for example; the cell or viral structural components or other polypeptides or nucleic acids commonly found associated with the polynucleotide. In representative embodiments an “isolated” nucleotide is enriched by at least about 10-fold, 100′-fold, 1000-fold, 10,000-fold or more as compared with the starting material.

Likewise, an “isolated” polypeptide means a polypeptide that is at least partially separated from at least some of the other components of the naturally occurring organism or virus, for example, the cell or viral structural components or other polypeptides or nucleic acids commonly found associated with the polypeptide. In representative embodiments an “isolated” polypeptide is enriched by at least about 10-fold, 100-fold, 1000-fold, 10,000-fold or more as compared with the starting material.

An “isolated cell” refers to a cell that is separated from other components with which it is normally associated in its natural state. For example, an isolated cell can be a cell in culture medium and/or a cell in a pharmaceutically acceptable carrier of this invention. Thus, an isolated cell can be delivered to and/or introduced into a subject. In some embodiments, an isolated cell can be a cell that is removed from a subject and manipulated as described herein ex vivo and then returned to the subject.

As used herein, by “isolate” or “purify” (or grammatical equivalents) a virus vector or virus particle or population of virus particles, it is meant that the virus vector or virus particle or population of virus particles is at least partially separated from at least some of the other components in the starting material. In representative embodiments an “isolated” or “purified” virus vector or virus particle or population of virus particles is enriched by at least about 10-fold, 100-fold, 1000-fold, 10,000-fold or more as compared with the starting material.

Unless indicated otherwise, “efficient transduction” or “efficient tropism,” or similar terms, can be determined by reference to a suitable control (e.g., at least about 10%, 15%, 20%, 25%, 30%, 35%, 40%, 45%, 50%, 55%, 60%, 65%, 70%, 75%, 80%, 85%, 90%, 95%, 100%, 125%, 150%, 175%, 200%, 250%, 300%, 350%, 400%, 500% or more of the transduction or tropism, respectively, of the control). In particular embodiments, the virus vector efficiently transduces or has efficient tropism for neuronal cells and cardiomyocytes. Suitable controls will depend on a variety of factors including the desired tropism and/or transduction profile.

A “therapeutic polypeptide” is a polypeptide that can alleviate, reduce, prevent, delay and/or stabilize symptoms that result from an absence or defect in a protein in a cell or subject and/or is a polypeptide that otherwise confers a benefit to a subject, e.g., enzyme replacement to reduce or eliminate symptoms of a disease, or improvement in transplant survivability or induction of an immune response.

By the terms “treat,” “treating” or “treatment of’ (and grammatical variations thereof) it is meant that the severity of the subject's condition is reduced, at least partially improved or stabilized and/or that some alleviation, mitigation, decrease or stabilization in at least one clinical symptom is achieved and/or there is a delay in the progression of the disease or disorder.

The terms “prevent,” “preventing” and “prevention” (and grammatical variations thereof) refer to prevention and/or delay of the onset of a disease, disorder and/or a clinical symptom(s) in a subject and/or a reduction in the severity of the onset of the disease, disorder and/or clinical symptom(s) relative to what would occur in the absence of the methods of the invention. The prevention can be complete, e.g., the total absence of the disease, disorder and/or clinical symptom(s). The prevention can also be partial, such that the occurrence of the disease, disorder and/or clinical symptom(s) in the subject and/or the severity of onset is substantially less than what would occur in the absence of the present invention.

A “treatment effective” amount as used herein is an amount that is sufficient to provide some improvement or benefit to the subject. Alternatively stated, a “treatment effective” amount is an amount that will provide some alleviation, mitigation, decrease or stabilization in at least one clinical symptom in the subject. Those skilled in the art will appreciate that the therapeutic effects need not be complete or curative, as long as some benefit is provided to the subject.

A “prevention effective” amount as used herein is an amount that is sufficient to prevent and/or delay the onset of a disease, disorder and/or clinical symptoms in a subject and/or to reduce and/or delay the severity of the onset of a disease, disorder and/or clinical symptoms in a subject relative to what would occur in the absence of the methods of the invention. Those skilled in the art will appreciate that the level of prevention need not be complete, as long as some preventative benefit is provided to the subject.

The terms “heterologous nucleotide sequence” and “heterologous nucleic acid molecule” are used interchangeably herein and refer to a nucleic acid sequence that is not naturally occurring in the virus. Generally, the heterologous nucleic acid molecule or heterologous nucleotide sequence comprises an open reading frame that encodes a polypeptide and/or nontranslated RNA of interest (e.g., for delivery to a cell and/or subject), for example CFTR.

As used herein, the terms “virus vector,” “viral vector”, “vector” or “gene delivery vector” refer to a manufactured construct comprising a virus capsid (e.g., AAV) that functions as a nucleic acid delivery vehicle, containing the packaged cassette of elements necessary for expression of the effector DNA (e.g., ITRs, promoter, intron(s), cDNA, poly A tail among others) and which comprises the vector. Alternatively, in some contexts, the term “vector” may be used to refer to the vector genome/vDNA alone.

An “rAAV vector genome” or “rAAV genome” is an AAV genome (i.e., vDNA) that comprises one or more heterologous nucleic acid sequences. rAAV vectors generally require only the inverted terminal repeat(s) (TR(s)) in cis to generate virus. All other viral sequences are dispensable and may be supplied in trans (Muzyczka, (1992) Curr. Topics Microbial. Immunol. 158:97). Typically, the rAAV vector genome will only retain the one or more TR sequence so as to maximize the size of the transgene that can be efficiently packaged by the vector. The structural and non-structural protein coding sequences may be provided in trans (e.g., from a vector, such as a plasmid, or by stably integrating the sequences into a packaging cell). In embodiments of the invention the rAAV vector genome comprises at least one ITR sequence (e.g., AAV TR sequence), optionally two ITRs (e.g., two AAV TRs), which typically will be at the 5′ and 3′ ends of the vector genome and flank the heterologous nucleic acid, but need not be contiguous thereto. The TRs can be the same or different from each other.

The term “terminal repeat” or “TR” includes any viral terminal repeat or synthetic sequence that forms a hairpin structure and functions as an inverted terminal repeat (i.e., an ITR that mediates the desired functions such as replication, virus packaging, integration and/or provirus rescue, and the like). The TR can be an AAV TR or a non-AAV TR. For example, a non-AAV TR sequence such as those of other parvoviruses (e.g., canine parvovirus (CPV), mouse parvovirus (MVM), human parvovirus B-19) or any other suitable virus sequence (e.g., the SV40 hairpin that serves as the origin of SV40 replication) can be used as a TR, which can further be modified by truncation, substitution, deletion, insertion and/or addition. Further, the TR can be partially or completely synthetic, such as the “double-D sequence” as described in U.S. Pat. No. 5,478,745 to Samulski et al.

An “AAV terminal repeat” or “AAV TR,” including an “AAV inverted terminal repeat” or “AAV ITR” may be from any AAV, including but not limited to serotypes 1, 2, 3, 4, 5, 6, 7, 8, 9, 10, 11 or 12 or any other AAV now known or later discovered. The two ITRs can be from the same or a different serotype. An AAV terminal repeat need not have the native terminal repeat sequence (e.g., a native AAV TR or AAV ITR sequence may be altered by insertion, deletion, truncation and/or missense mutations), as long as the terminal repeat mediates the desired functions, e.g., replication, virus packaging, integration, and/or provirus rescue, and the like.

AAV proteins VP1, VP2 and VP3 are capsid proteins that interact together to form an AAV capsid of an icosahedral symmetry. VP1.5 is an AAV capsid protein described in US Publication No. 2014/0037585. However, the capsid's proteins can be modified and from any AAV serotype. In one embodiment, the capsid protein is from the same serotype as at least one AAV ITR. In another embodiment, at least one ITR and a capsid protein is from a different serotype.

The virus vectors of the invention can further be “targeted” virus vectors (e.g., having a directed tropism) and/or a “hybrid” parvovirus (i.e., in which the viral TRs and viral capsid are from different parvoviruses) as described in international patent publication WO 00/28004 and Chao et al., (2000) Molecular Therapy 2:619.

The virus vectors of the invention can further be duplexed parvovirus particles as described in international patent publication WO 01/92551 (the disclosure of which is incorporated herein by reference in its entirety). Thus, in some embodiments, double stranded (duplex) genomes can be packaged into the virus capsids of the invention.

Further, the viral capsid or genomic elements can contain other modifications, including insertions, deletions and/or substitutions.

A “chimeric’ capsid protein as used herein means an AAV capsid protein that has been modified by substitutions in one or more (e.g., 2, 3, 4, 5, 6, 7, 8, 9, 10, etc.) amino acid residues in the amino acid sequence of the capsid protein relative to wild type, as well as insertions and/or deletions of one or more (e.g., 2, 3, 4, 5, 6, 7, 8, 9, 10, etc.) amino acid residues in the amino acid sequence relative to wild type. In some embodiments, complete or partial domains, functional regions, epitopes, etc., from one AAV serotype can replace the corresponding wild type domain, functional region, epitope, etc. of a different AAV serotype, in any combination, to produce a chimeric capsid protein of this invention. Production of a chimeric capsid protein can be carried out according to protocols well known in the art and a significant number of chimeric capsid proteins are described in the literature as well as herein that can be included in the capsid of this invention.

As used herein, the term “haploid AAV” shall mean that AAV as described in PCT/US18/22725, which is incorporated herein.

The term a “hybrid” AAV vector or parvovirus refers to a rAAV vector where the viral TRs or ITRs and viral capsid are from different parvoviruses. Hybrid vectors are described in international patent publication WO 00/28004 and Chao et al., (2000) Molecular Therapy 2:619. For example, a hybrid AAV vector typically comprises the adenovirus 5′ and 3′ cis ITR sequences sufficient for adenovirus replication and packaging (i.e., the adenovirus terminal repeats and PAC sequence).

The term “polyploid AAV” refers to a AAV vector which is composed of capsids from two or more AAV serotypes, e.g., and can take advantages from individual serotypes for higher transduction but not in certain embodiments eliminate the tropism from the parents.

As used herein, the term “amino acid” encompasses any naturally occurring amino acid, modified forms thereof, and synthetic amino acids.

Additional patents incorporated for reference herein that are related to, disclose or describe an AAV or an aspect of an AAV, including the DNA vector that includes the gene of interest to be expressed are: U.S. Pat. Nos. 6,491,907; 7,229,823; 7,790,154; 7,201898; 7,071,172; 7,892,809; 7,867,484; 8,889,641; 9,169,494; 9,169,492; 9,441,206; 9,409,953; and, 9,447,433; 9,592,247; and, 9,737,618.

### II. rAAV Genome Elements

As disclosed herein, one aspect of the technology relates to a rAAV vector comprising a capsid, and within its capsid, a nucleotide sequence referred to as the “rAAV vector genome”. The rAAV vector genome (also referred to as “rAAV genome) includes multiple elements, including, but not limited to two inverted terminal repeats (ITRs, e.g., the 5′-ITR and the 3′-ITR), and located between the ITRs are additional elements, including a promoter, a heterologous gene and a poly-A tail.

In some embodiments, the rAAV genome disclosed herein comprises a 5′ ITR and 3′ ITR sequence, and located between the 5′ITR and the 3′ ITR, a promoter, e.g., a lung specific promoter sequence, which operatively linked to a heterologous a nucleic acid encoding a therapeutic protein, where the heterologous nucleic acid sequence can further comprise one or more of the following elements: an intron sequence, a nucleic acid encoding a secretory signal peptide, and a poly A sequence.

### F. Promoters

In some embodiments, to achieve appropriate levels of a therapeutic protein, the rAAV genotype comprises a promoter. A suitable promoter can be selected from any of a number of promoters known to one of ordinary skill in the art. In some embodiments, a promoter is a cell-type specific promotor. In a further embodiment, a promoter is an inducible promotor. In an embodiment, a promotor is located upstream 5′ and is operatively linked to the heterologous nucleic acid sequence. In some embodiments, the promotor is a liver cell-type specific promotor, a heart muscle cell-type specific promoter, a neuron cell-type specific promoter, a nerve cell-type specific promoter, a muscle cell-type specific promoter, or a lung-specific promoter or another cell-type specific promoter.

In some embodiments, a constitutive promoter can be selected from a group of constitutive promoters of different strengths and tissue specificity. Some examples of these promoters are set forth in Table 6. A viral vector such as rAAV vector genome can include one or more constitutive promoters, such as viral promoters or promoters from mammalian genes that are generally active in promoting transcription. Examples of constitutive viral promoters are: Herpes Simplex virus (HSV) promoter, thymidine kinase (TK) promoter. Rous Sarcoma Virus (RSV) promoter, Simian Virus 40 (SV40) promoter, Mouse Mammary Tumor Virus (MMTV) promoter, Ad EIA promoter and cytomegalovirus (CMV) promoters. Examples of constitutive mammalian promoters include various housekeeping gene promoters, as exemplified by the β-actin promoter and the chicken beta-actin (CB) promoter, wherein the CB promoter has proven to be a particularly useful constitutive promoter for expressing CFTR.

In an embodiment, the promoter is a tissue-specific promoter such as a lung-specific promoter, including but not limited to promoter sequences, including the lung-specific SP-C promoter that mediates strong and lung-specific transgene expression as described in Degiulio J V et al. Gene Ther. 2010 April; 17(4):541-549.ID

In an embodiment, a promoter is an inducible promoter. Examples of suitable inducible promoters include those from genes such as cytochrome P450 genes, heat shock protein genes, metallothionein genes, and hormone-inducible genes, including the estrogen gene promoter. Another example of an inducible promoter is the tetVP16 promoter that is responsive to tetracycline.

Promoters in a rAAV genome according to the disclosure herein include, but are not limited to neuron-specific promoters, such as synapsin 1 (SYN) promoter; muscle creatine kinase (MCK) promoters; and desmin (DES) promoters. In one embodiment, the AAV-mediated expression of heterologous nucleic acids (such as a human CFTR) can be achieved in neurons via a Synapsin promoter or in skeletal muscles via an MCK promoter. Other promoters that can be used include, EF, B19p6, CAG, neurone specific enolase gene promoter; chicken beta-actin/CMV hybrid promoter; platelet derived growth factor gene promoter; bGH, EF1a, CamKIIa, GFAP, RPE, ALB, TBG, MBP, MCK, TNT, aMHC, GFP, RFP, mCherry, CFP and YFP promoters.

TABLE 1
Exemplary promoters.

| Promoter | Description/Loci | | | | | |
|---|---|---|---|---|---|---|
| name | (plasmid names) | Size | | Target cell type | notes | references |
| CMV | Cytomegalovirus | ~600 | bps | most cell types | Can undergo | Zolotukhin et al. |
| | immediate early | | | | silencing in- | 1996; Zolotukhin |
| | promoter(pTR-UF5) | | | | vivo | et a. 1999 |
| CBAaka: CB, | Hybrid CMV/Chicken | 1720 | bps | most cell types | Contains | Acland et al. |
| CAG | beta actin | | | | 381 bps version | 2001; Cideciyan |
| | promoter(pTR-UF11, | | | | of CMV i.e. | et al. 2008 |
| | pTR-UF-SB) | | | | enhancer | |
| smCBAaka: | Truncated CBA | 953 | bps | most cell types | Chimeric | Pang et al. 2008; |
| small CBA | promoter | | | | Intron | |
| | | | | | collapsed. Used | |
| | | | | | for ScAAV | |
| MOPS aka: | Proximal murine | ~500 | bps | Photoreceptors, | | Flannery et al. |
| mOP, mRHO, | rhodopsin promoter | | | primarily rods | | 1997; |
| MOPS500 | | | | | | |
| GRK1aka: | Human rhodopsin | 292 | bps | Photoreceptor, | Does not | Khani et al. 2007; |
| hGRK, hRK, | kinase 1 promoter | | | rods and cones | transduce cones | Boye et al. 2010; |
| RK1 | | | | (mouse and primate) | in dog | Boye et al. 2012 |
| IRBPaka: | Human inter- | 241 | bps | Photoreceptors, | | Beltran et al. |
| hIRBP241 | photoreceptor retinoid | | | rods and cones | | 2012 |
| | binding | | | (mouse and dog) | | |
| | protein/Retinol-binding | | | | | |
| | protein 3 | | | | | |
| PR2.1aka: | Human red opsin | ~2100 | bps | L and M cones | | Alexander et al. |
| CHOPS2053 | promoter | | | | | 2007; Mancuso et |
| | | | | | | al. 2009; |
| | | | | | | Komaromy et al. |
| | | | | | | 2010 |
| IRBP/GNAT2 | hIRBP enhancer fused | 524 | bps | L/M and S cones | Efficiently | |
| | to cone transducin | | | | transduces all | |
| | alpha promoter | | | | classes of cones | |
| VMD2Aka: | Human vitelliform | 625 | bps | RPE | Highly | Deng et al. 2012 |
| BEST1 | macular | | | | selective for | |
| | dystrophy/Bestrophin 1 | | | | RPE | |
| | promoter | | | | | |
| VEcadaka: | VE-cadherin/Cadherin | 2530 | bps | Vascular | | Cai et al. 2011; |
| VEcadherin | 5 (CDH5)/CD144 | | | endothelial cells | | Qi et al. 2012 |
| | promoter | | | | | |
| SP-B | Surfactant protein B | | | Bronchiolar and | | Strayer M. et al. |
| | | | | alveolar | | 2002; Venkatesh |
| | | | | epithelial cells of | | V C et al. 1995 |
| | | | | the lung. | | |

### H. Poly-A

In some embodiments, an viral vector genome, e.g., a rAAV vector genome includes at least one poly-A tail that is located 3′ and downstream from the heterologous nucleic acid gene encoding the in one embodiment, a CFTR fusion polypeptide. In some embodiments, the polyA signal is 3′ of a stability sequence or CS sequence as defined herein. Any polyA sequence can be used, including but not limited to hGH poly A, synpA polyA and the like. In some embodiments, the polyA is a synthetic polyA sequence. In some embodiments, the rAAV vector genome comprises two poly-A tails, e.g., a hGH poly A sequence and another polyA sequence, where a spacer nucleic acid sequence is located between the two poly A sequences. In some embodiments, the first poly A sequence is a hGH poly A sequence and the second poly A sequence is a synthetic sequence, or vice versa—that is, in alternative embodiments, the first poly A sequence is a synthetic poly A sequence and the second poly A sequence is a hGH polyA sequence. An exemplary poly A sequence is, for example, hGH poly A sequence, or a poly A nucleic acid sequence having at least sequence at least 80%, 85%, 90%, 95%, 96%, 97%, 98% or 99% nucleotide sequence identity to the hGH poly A sequence. In some embodiments, the hGHpoly sequence encompassed for use is described in Anderson et al. J. Biol. Chem 264(14); 8222-8229, 1989 (See, e.g. p. 8223, 2nd column, first paragraph) which is incorporated herein in its entirety by reference.

In some embodiments, a poly-A tail can be engineered to stabilize the RNA transcript that is transcribed from an rAAV vector genome, including a transcript for a heterologous gene, and in alternative embodiments, the poly-A tail can be engineered to include elements that are destabilizing.

In an embodiment, a poly-A tail can be engineered to become a destabilizing element by altering the length of the poly-A tail. In an embodiment, the poly-A tail can be lengthened or shortened. In a further embodiment, the 3′ untranslated region that lies between the heterologous gene, in one embodiment a CFTR gene, and the poly-A tail can be lengthened or shortened to alter the expression levels of the heterologous gene or alter the final polypeptide that is produced. In some embodiments, the 3′ untranslated region comprises GAA 3′ UTR.

In another embodiment, a destabilizing element is a microRNA (miRNA) that has the ability to silence (repress translation and promote degradation) the RNA transcripts the miRNA bind to that encode a heterologous gene. Modulation of the expression of a heterologous gene, e.g., IGF2(V43M)-CFTRfusion polypeptide, can be undertaken by modifying, adding or deleting seed regions within the poly-A tail to which the miRNA bind. In an embodiment, addition or deletion of seed regions within the poly-A tail can increase or decrease expression of a protein, e.g., IGF2(V43M)-CFTRfusion polypeptide, encoded by a heterologous gene in an rAAV vector genome. In a further embodiment, such increase or decrease in expression resultant from the addition or deletion of seed regions is dependent on the cell type transduced by the AAV containing an rAAV vector genome.

In another embodiment, seed regions can also be engineered into the 3′ untranslated regions located between the heterologous gene and the poly-A tail. In a further embodiment, the destabilizing agent can be an siRNA. The coding region of the siRNA can be included in an rAAV vector genome and is generally located downstream, 3′ of the poly-A tail.

### I. Terminal Repeats

The rAAV genome as disclosed here comprises AAV ITRs that have desirable characteristics and can be designed to modulate the activities of, and cellular responses to vectors that incorporate the ITRs. In another embodiment, the AAV ITRs are synthetic AAV ITRs that has desirable characteristics and can be designed to manipulate the activities of and cellular responses to vectors comprising one or two synthetic ITRs, including, as set forth in U.S. Pat. No. 9,447,433, which is incorporated herein by reference. Lentiviruses have long terminal repeats LTRs that also assist in packaging.

The AAV ITRs for use in the rAAV and the LTRs for use with lentiviruses such as HIV flank the transgene genome as disclosed herein may be of any serotype suitable for a particular application. In some embodiments, the AAV vector genome is flanked by AAV ITRs. In some embodiments, the rAAV vector genome is flanked by AAV ITRs, wherein an ITR comprises a full length ITR sequence, an ITR with sequences comprising CPG islands removed, an ITR with sequences comprising CPG sequences added, a truncated ITR sequence, an ITR sequence with one or more deletions within an ITR, an ITR sequence with one or more additions within an ITR, or a combination of comprising any portion of the aforementioned ITRs linked together to form a hybrid ITR.

In order to facilitate long term expression, in an embodiment, the polynucleotide encoding GAA is interposed between an AAV inverted terminal repeats (ITRs) (e.g., the first or 5′ and second 3′ AAV ITRs) or an LTR, e.g. an HIV LTR. AAV ITRs are found at both ends of a WT rAAV vector genome, and serve as the origin and primer of DNA replication. ITRs are required in cis for AAV DNA replication as well as for rescue, or excision, from prokaryotic plasmids. In an embodiment, the AAV ITR sequences that are contained within the nucleic acid of the rAAV genome can be derived from any AAV serotype (e.g. 1, 2, 3, 3b, 4, 5, 6, 7, 8, 9, and 10) or can be derived from more than one serotype, including combining portions of two or more AAV serotypes to construct an ITR. In an embodiment, for use in the rAAV vector, including an rAAV vector genome, the first and second ITRs should include at least the minimum portions of a WT or engineered ITR that are necessary for packaging and replication. In some embodiments, an rAAV vector genome is flanked by AAV ITRs.

In some embodiments, the rAAV vector genome comprises at least one AAV ITR, wherein said ITR comprises, consists essentially of, or consists of; (a) an AAV rep binding element; (b) an AAV terminal resolution sequence; and (c) an AAV RBE (Rep binding element); wherein said ITR does not comprise any other AAV ITR sequences. In another embodiment, elements (a), (b), and (c) are from an AAV9 ITR and the ITR does not comprise any other AAV9 ITR sequences. In a further embodiment, elements (a), (b) and (c) are from any AAV ITR, including but not limited to AAV2, AAV8 and AAV9. In some embodiments, the polynucleotide comprises two synthetic ITRs, which may be the same or different.

In some embodiments, the polynucleotide in the rAAV vector, including an rAAV vector genome comprises two ITRs, which may be the same or different. The three elements in the ITR have been determined to be sufficient for ITR function. This minimal functional ITR can be used in all aspects of AAV vector production and transduction. Additional deletions may define an even smaller minimal functional ITR. The shorter length advantageously permits the packaging and transduction of larger transgenic cassettes.

In another embodiment, each of the elements that are present in a synthetic ITR can be the exact sequence as exists in a naturally occurring AAV ITR (the WT sequence) or can differ slightly (e.g., differ by addition, deletion, and/or substitution of 1, 2, 3, 4, 5 or more nucleotides) so long as the functioning of the elements of the AAV ITR continue to function at a level sufficient to are not substantially different from the functioning of these same elements as they exist in a naturally occurring AAV ITR.

In a further embodiment, rAAV vector, including an rAAV vector genome can comprise, between the ITRs, one or more additional non-AAV cis elements, e.g., elements that initiate transcription, mediate enhancer function, allow replication and symmetric distribution upon mitosis, or alter the persistence and processing of transduced genomes. Such elements are well known in the art and include, without limitation, promoters, enhancers, chromatin attachment sequences, telomeric sequences, cis-acting microRNAs (miRNAs), and combinations thereof.

In another embodiment, an ITR exhibits modified transcription activity relative to a naturally occurring ITR, e.g., ITR9 from AAV9. It is known that the ITR9 sequence inherently has promoter activity. It also inherently has termination activity, similar to a poly(A) sequence. The minimal functional ITR of the present invention exhibits transcription activity as shown in the examples, although at a diminished level relative to ITR2. Thus, in some embodiments, the ITR is functional for transcription. In other embodiments, the ITR is defective for transcription. In certain embodiments, the ITR can act as a transcription insulator, e.g., preventing transcription of a transgenic cassette present in the vector when the vector is integrated into a host chromosome.

One aspect of the invention relates to an rAAV vector genome comprising at least one synthetic AAV ITR, wherein the nucleotide sequence of one or more transcription factor binding sites in the ITR is deleted and/or substituted, relative to the sequence of a naturally occurring AAV ITR such as ITR2. In some embodiments, it is the minimal functional ITR in which one or more transcription factor binding sites are deleted and/or substituted. In some embodiments at least 1 transcription factor binding site is deleted and/or substituted, e.g., at least 5 or more or 10 or more transcription factor binding sites, e.g., at least 1, 2, 3, 4, 5, 6, 7, 8, 9, 10, 11, 12, 13, 14, 15, 16, 17, 18, 19, 20, or 21 transcription factor binding sites.

Another embodiment, a rAAV vector, including an rAAV vector genome as described herein comprises a polynucleotide comprising at least one synthetic AAV ITR, wherein one or more CpG islands (a cytosine base followed immediately by a guanine base (a CpG) in which the cytosines in such arrangement tend to be methylated) that typically occur at, or near the transcription start site in an ITR are deleted and/or substituted. In an embodiment, deletion or reduction in the number of CpG islands can reduce the immunogenicity of the rAAV vector. This results from a reduction or complete inhibition in TLR-9 binding to the rAAV vector DNA sequence, which occurs at CpG islands. It is also well known that methylation of CpG motifs results in transcriptional silencing. Removal of CpG motifs in the ITR is expected to result in decreased TLR-9 recognition and/or decreased methylation and therefore decreased transgene silencing. In some embodiments, it is the minimal functional ITR in which one or more CpG islands are deleted and/or substituted. In an embodiment, AAV ITR2 is known to contain 16 CpG islands of which one or more, or all 16 can be deleted.

In some embodiments, at least 1 CpG motif is deleted and/or substituted, e.g., at least 4 or more or 8 or more CpG motifs, e.g., at least 1, 2, 3, 4, 5, 6, 7, 8, 9, 10, 11, 12, 13, 14, 15, or 16 CpG motifs. The phrase “deleted and/or substituted” as used herein means that one or both nucleotides in the CpG motif is deleted, substituted with a different nucleotide, or any combination of deletions and substitutions.

In another embodiment, the synthetic ITR comprises, consists essentially of, or consists of one of the nucleotide sequences listed below. In other embodiments, the synthetic ITR comprises, consist essentially of, or consist of a nucleotide sequence that is at least 80% identical, e.g., at least 85%, 90%, 95%, 96%, 97%, 98%, or 99% identical to one of the nucleotide sequences listed below.

MH-257
(SEQ ID NO: 300)
AGGAACCCCTAGTGATGGAGTTGGCCACTCCCTCTCTGCGCGCTCGCTC
GCTCACTGAGGCAATTTGATAAAAATCGTCAAATTATAAACAGGCTTTG
CCTGTTTAGCCTCAGTGAGCGAGCGAGCGCGCAGAGAGGGAGTGGCCAA
CTCCATCACTAGGGGTTCCT
MH-258
(SEQ ID NO: 301)
AGGAACCCCTAGTGATGGAGTTGGCCACTCCCTCTCTGCGCGCTCGCTC
GCTCACTGAGGGATAAAAATCCAGGCTTTGCCTGCCTCAGTGAGCGAGC
GAGCGCGCAGAGAGGGAGTGGCCAACTCCATCACTAGGGGTTCCT
MH Delta 258
(SEQ ID NO: 302)
AGGAACCCCTAGTGATGGAGTTGGCCACTCCCTCTCTGCGCGCTCGCTC
GCTCACTGAGGGATAAAAATCCAGGCTTTGCCTGCCTCAGTGAGCGAGC
GAGCGCGCAGAGAGGGAGTGGCCAACTCCATCACTAGGGGTTCCT
MH Telomere-1 ITR
(SEQ ID NO: 303)
AGGAACCCCTAGTGATGGAGTTGGCCACTCCCTCTCTGGGATTGGGATT
GCGCGCTCGCTCGCGGGATTGGGATTGGGATTGGGATTGGGATTGGGAT
TGATAAAAATCAATCCCAATCCCAATCCCAATCCCAATCCCAATCCCGC
GAGCGAGCGCGCAATCCCAATCCCAGAGAGGGAGTGGCCAACTCCATCA
CTAGGGGTTCCT
MH Telomere-2 ITR
(SEQ ID NO: 304)
AGGAACCCCTAGTGATGGAGTTGGCCACTCCCTCTCTGCGCGCTCGCTC
GCTCGGGATTGGGATTGGGATTGGGATTGGGATTGGGATTGATAAAAAT
CAATCCCAATCCCAATCCCAATCCCAATCCCAATCCCGCGAGCGAGCGC
GCAGGAGAGGGAGTGGCCAACTCCATCACTAGGGGTTCCTAAGCTTATT
ATA
MH PolII 258 ITR
(SEQ ID NO: 305)
AGGAACCCCTAGTGATGGAGTTGGCCACTCCCTCTCTGCGCGCTCGCTC
GCTCACTGAGGGCGCCTATAAAGATAAAAATCCAGGCTTTGCCTGCCTC
AGTTAGCGAGCGAGCGCGCAGAGAGGGAGTGGCCAACTCCATCACTAGG
GGTTCCT
MH 258 Delta D conservative
(SEQ ID NO: 306)
CTAGTGATGGAGTTGGCCACTCCCTCTCTGCGCGCTCGCTCGCTCACTG
AGGGATAAAAATCCAGGCTTTGCCTGCCTCAGTGAGCGAGCGAGCGCGC
AGAGAGGGAGTGGCCAACTCCATCACTAG

In certain embodiments, a rAAV vector genome as described herein comprises a synthetic ITR that is capable of producing AAV virus particles that can transduce host cells. Such ITRs can be used, for example, for viral delivery of heterologous nucleic acids. Examples of such ITRs include MH-257, MH-258, and MH Delta 258 listed above.

In other embodiments, a rAAV vector genome as described herein containing a synthetic ITR is not capable of producing AAV virus particles. Such ITRs can be used, for example, for non-viral transfer of heterologous nucleic acids. Examples of such ITRs include MH Telomere-1, MH Telomere-2, and MH Pol II 258 listed above.

In a further embodiment, an rAAV vector genome as described herein comprising the synthetic ITR of the invention further comprises a second ITR which may be the same as or different from the first ITR. In one embodiment, an rAAV vector genome further comprises a heterologous nucleic acid, e.g., a sequence encoding a protein or a functional RNA. In an additional embodiment, a second ITR cannot be resolved by the Rep protein, i.e., resulting in a double stranded viral DNA.

In an embodiment, an rAAV vector genome comprises a polynucleotide comprising a synthetic ITR of the invention. In a further embodiment, the viral vector can be a parvovirus vector, e.g., an AAV vector. In another embodiment, a recombinant parvovirus particle (e.g., a recombinant AAV particle) containing a vector genome having at least one synthetic ITR.

Another embodiment of the invention relates to a method of increasing the transgenic DNA packaging capacity of an AAV capsid, comprising generating an rAAV vector genome comprising at least one synthetic AAV ITR, wherein said ITR comprises: (a) an AAV rep binding element; (b) an AAV terminal resolution sequence; and (c) an AAV RBE element; wherein said ITR does not comprise any other AAV ITR sequences.

A further embodiment of the invention relates to a method of altering the cellular response to infection by an rAAV vector genome, comprising generating an rAAV vector genome comprising at least one synthetic ITR, wherein the nucleotide sequence of one or more transcription factor binding sites in said ITR is deleted and/or substituted, and further wherein an rAAV vector genome comprises at least one synthetic ITR that produces an altered cellular response to infection.

An additional embodiment of the invention relates to a method of altering the cellular response to infection by an rAAV vector genome, comprising generating an rAAV vector genome comprising at least one synthetic ITR, wherein one or more CpG motifs in said ITR are deleted and/or substituted, wherein the vector comprising at least one synthetic ITR produces an altered cellular response to infection.

### III. Vectors And Virions

A targeted viral vector can be any viral vector useful for gene therapy, e.g., including but not limited to lentivirus, adenovirus (Ad), adeno-associated viruses (AAV), HSV etc.

The choice of delivery vector can be made based on a number of factors known in the art, including age and species of the target host, in vitro vs. in vivo delivery, level and persistence of expression desired, intended purpose (e.g., for therapy or polypeptide production), the target cell or organ, route of delivery, size of the isolated nucleic acid, safety concerns, and the like.

Suitable vectors include virus vectors (e.g., retrovirus, alphavirus; vaccinia virus; adenovirus, adeno-associated virus, or herpes simplex virus), lipid vectors, poly-lysine vectors, synthetic polyamino polymer vectors that are used with nucleic acid molecules, such as plasmids, and the like.

Any viral vector that is known in the art can be used in the present invention. Examples of such viral vectors include, but are not limited to vectors derived from: Adenoviridae; Birnaviridae; Bunyaviridae; Caliciviridae, Capillovirus group; Carlavirus group; Carmovirus virus group; Group Caulimovirus; Closterovirus Group; Commelina yellow mottle virus group; Comovirus virus group; Coronaviridae; PM2 phage group; Corcicoviridae; Group Cryptic virus; group Cryptovirus; Cucumovirus virus group Family ([PHgr]6 phage group; Cysioviridae; Group Carnation ringspot; Dianthovirus virus group; Group Broad bean wilt; Fabavirus virus group; Filoviridae; Flaviviridae; Furovirus group; Group Germinivirus; Group Giardiavirus; Hepadnaviridae; Herpesviridae; Hordeivirus virus group; Illarvirus virus group; lnoviridae; Iridoviridae; Leviviridae; Lipothrixviridae; Luteovirus group; Marafivirus virus group; Maize chlorotic dwarf virus group; icroviridae; Myoviridae; Necrovirus group; Nepovirus virus group; Nodaviridae; Orthomyxoviridae; Papovaviridae; Paramyxoviridae; Parsnip yellow fleck virus group; Partitiviridae; Parvoviridae; Pea enation mosaic virus group; Phycodnaviridae; Picomaviridae; Plasmaviridae; Prodoviridae; Polydnaviridae; Potexvirus group; Potyvirus; Poxviridae; Reoviridae; Retroviridae; Rhabdoviridae; Group Rhizidiovirus; Siphoviridae; Sobemovirus group; SSV 1-Type Phages; Tectiviridae; Tenuivirus; Tetraviridae; Group Tobamovirus; Group Tobravirus; Togaviridae; Group Tombusvirus; Group Tobovirus; Totiviridae; Group Tymovirus; and Plant virus satellites.

Protocols for producing recombinant viral vectors and for using viral vectors for nucleic acid delivery can be found in Bouard, D. et al, Br J. Pharmacol 2009 May, 157(2) 153-165 “Viral Vectors: from virology to transgene expression”, Current Protocols in Molecular Biology, Ausubel, F. M. et al. (eds.) Greene Publishing Associates, (1989) and other standard laboratory manuals (e.g., Vectors for Gene Therapy. In: Current Protocols in Human Genetics. John Wiley and Sons, Inc.: 1997).

Particular examples of viral vectors for the delivery of nucleic acids include, for example, retrovirus, lentivirus, adenovirus, AAV and other parvoviruses, herpes virus, and poxvirus vectors. Lentiviruses are a type of retrovirus that can infect both dividing and non-dividing cells. They include human immunodeficiency virus (HIV), simian immunodeficiency virus (SIV), feline immunodeficiency virus (FIV), bovine immunodeficiency virus (BIV). The transgene is flanked by LTRs that can be the same or different, synthetic, chimerics, etc. In addition elements like tat and rev can enhance expression of the transgene.

Retroviruses also include γ-retroviral vectors such as maurine leukemia virus (MLV) wherein the transgene is also flanked on both sides by LTRs.

The term “parvovirus” as used herein encompasses the family Parvoviridae, including autonomously-replicating parvoviruses and dependoviruses. The autonomous parvoviruses include members of the genera Parvovirus, Erythrovirus, Densovirus, Iteravirus, and Contravirus. Exemplary autonomous parvoviruses include, but are not limited to, minute virus of mouse, bovine parvovirus, canine parvovirus, chicken parvovirus, feline panleukopenia virus, feline parvovirus, goose parvovirus, H1 parvovirus, muscovy duck parvovirus, and B19 virus, and any other virus classified by the International Committee on Taxonomy of Viruses (ICTV) as a parvovirus.

Other autonomous parvoviruses are known to those skilled in the art. See, e.g., BERNARD N. FIELDS et al., VIROLOGY, volume 2, chapter 69 (4th ed., Lippincott-Raven Publishers).

The genus Dependovirus contains the adeno-associated viruses (AAV), including but not limited to, AAV type 1, AAV type 2, AAV type 3, AAV type 4, AAV type 5, AAV type 6, AAV type 7, AAV type 8, AAV type 9, AAV type 10, AAV type 11, AAV type 12, AAV type 13, avian AAV, bovine AAV, canine AAV, equine AAV, and ovine AAV, and any other virus classified by the International Committee on Taxonomy of Viruses (ICTV) as a dependovirus (e.g., AAV). See, e.g., BERNARD N. FIELDS et al., VIROLOGY, volume 2, chapter 69 (4th ed., Lippincott-Raven Publishers).

In particular embodiments, the delivery vector comprises an AAV capsid including but not limited to a capsid from AAV type 1, AAV type 2, AAV type 3, AAV type 4, AAV type 5, AAV type 6, AAV type 7 or AAV type 8, AAV type 9, AAV type 10, AAV type 11, AAV type 12, AAV type 13. The capsid proteins can be from the same or different serotypes.

Table 2 describe exemplary AAV Serotypes and exemplary published corresponding capsid sequence that can be used as the AAV capsid in the rAAV vector described herein, or with any combination with wild type capsid proteins and/or other chimeric or variant capsid proteins now known or later identified and each is incorporated herein.

TABLE 2
AAV Serotypes and exemplary published corresponding capsid sequence
The sequences listed in this table are known in the art and
are incorporated hereby by reference only in their entirety.

| Serotype and where capsid sequence is published | Serotype and where capsid sequence is published |
|---|---|
| AAV3.3b See US20030138772 SEQ ID NO: 72 | AAV3-3 See US20150315612 SEQ ID NO: 200 |
| AAV3-3 See US20150315612 SEQ ID NO: 217 | AAV3a See U.S. Pat. No. 6,156,303 SEQ ID NO: 5 |
| AAV3a See U.S. Pat. No. 6,156,303 SEQ ID NO: 9 | AAV3b See U.S. Pat. No. 6,156,303 SEQ ID NO: 6 |
| AAV3b See U.S. Pat. No. 6,156,303 SEQ ID NO: 10 | AAV3b See U.S. Pat. No. 6,156,303 SEQ ID NO: 1 |
| AAV4 See US20140348794 SEQ ID NO: 17 | AAV4 See US20140348794 SEQ ID NO: 5 |
| AAV4 See US20140348794 SEQ ID NO: 3 | AAV4 See US20140348794 SEQ ID NO: 14 |
| AAV4 See US20140348794 SEQ ID NO: 15 | AAV4 See US20140348794 SEQ ID NO: 19 |
| AAV4 See US20140348794 SEQ ID NO: 12 | AAV4 See US20140348794 SEQ ID NO: 13 |
| AAV4 See US20140348794 SEQ ID NO: 7 | AAV4 See US20140348794 SEQ ID NO: 8 |
| AAV4 See US20140348794 SEQ ID NO: 9 | AAV4 See US20140348794 SEQ ID NO: 2 |
| AAV4 See US20140348794 SEQ ID NO: 10 | AAV4 See US20140348794 SEQ ID NO: 11 |
| AAV4 See US20140348794 SEQ ID NO: 18 | AAV4 See US20030138772 SEQ ID NO: 63, US20160017295 |
| | SEQ ID NO: See US20140348794 SEQ ID NO: 4 |
| | AAV4 See US20140348794 SEQ ID NO: 16 |
| AAV4 See US20140348794 SEQ ID NO: 20 | AAV4 See US20140348794 SEQ ID NO: 6 |
| AAV4 See US20140348794 SEQ ID NO: 1 | AAV42.2 See US20030138772 SEQ ID NO: 9 |
| AAV42.2 See US20030138772 SEQ ID NO: 102 | AAV42.3b See US20030138772 SEQ ID NO: 36 |
| AAV42.3B See US20030138772 SEQ ID NO: 107 | AAV42.4 See US20030138772 SEQ ID NO: 33 |
| AAV42.4 See US20030138772 SEQ ID NO: 88 | AAV42.8 See US20030138772 SEQ ID NO: 27 |
| AAV42.8 See US20030138772 SEQ ID NO: 85 | AAV43.1 See US20030138772 SEQ ID NO: 39 |
| AAV43.1 See US20030138772 SEQ ID NO: 92 | AAV43.12 See US20030138772 SEQ ID NO: 41 |
| AAV43.12 See US20030138772 SEQ ID NO: 93 | AAV8 See US20150159173 SEQ ID NO: 15 |
| AAV8 See US20150376240 SEQ ID NO: 7 | AAV8 See US20030138772 SEQ ID NO: 4, US20150315612 |
| | SEQ ID NO: 182 |
| | AAV8 See US20030138772 SEQ ID NO: 95, US20140359799 |
| | SEQ ID NO: 1, US20150159173 SEQ ID NO: 31, |
| | US20160017295 SEQ ID NO: 8, U.S. Pat. No. 7,198,951 |
| | SEQ ID NO: 7, US20150315612 SEQ ID NO: 223 |
| AAV8 See US20150376240 SEQ ID NO: 8 | AAV8 See US20150315612 SEQ ID NO: 214 |
| AAV-8b See US20150376240 SEQ ID NO: 5 | AAV-8b See US20150376240 SEQ ID NO: 3 |
| AAV-8h See US20150376240 SEQ ID NO: 6 | AAV-8h See US20150376240 SEQ ID NO: 4 |
| AAV9 See US20030138772 SEQ ID NO: 5 | AAV9 See U.S. Pat. No. 7,198,951 SEQ ID NO: 1 |
| AAV9 See US20160017295 SEQ ID NO: 9 | AAV9 See US20030138772 SEQ ID NO: 100, U.S. Pat. No. |
| | 7,198,951 SEQ ID NO: 2 |
| | AAV9 See U.S. Pat. No. 7,198,951 SEQ ID NO: 3 |
| AAV9 (AAVhu.14) See US20150315612 SEQ ID NO: 3 | AAV9 (AAVhu.14) See US20150315612 SEQ ID NO: 123 |
| AAVA3.1 See US20030138772 SEQ ID NO: 120 | AAVA3.3 See US20030138772 SEQ ID NO: 57 |
| AAVA3.3 See US20030138772 SEQ ID NO: 66 | AAVA3.4 See US20030138772 SEQ ID NO: 54 |
| AAVA3.4 See US20030138772 SEQ ID NO: 68 | AAVA3.5 See US20030138772 SEQ ID NO: 55 |
| AAVA3.5 See US20030138772 SEQ ID NO: 69 | AAVA3.7 See US20030138772 SEQ ID NO: 56 |
| AAVA3.7 See US20030138772 SEQ ID NO: 67 | AAV29. See (AAVbb. l) 161 US20030138772 SEQ ID NO: 11 |
| AAVC2 See US20030138772 SEQ ID NO: 61 | AAVCh.5 See US20150159173 SEQ ID NO: 46, US20150315612 |
| | SEQ ID NO: 234 |
| | AAVcy.2 (AAV13.3) See US20030138772 SEQ ID NO: 15 |
| AAV24.1 See US20030138772 SEQ ID NO: 101 | AAVcy.3 (AAV24.1) See US20030138772 SEQ ID NO: 16 |
| AAV27.3 See US20030138772 SEQ ID NO: 104 | AAVcy.4 (AAV27.3) See US20030138772 SEQ ID NO: 17 |
| AAVcy.5 See US20150315612 SEQ ID NO: 227 | AAV7.2 See US20030138772 SEQ ID NO: 103 |
| AAVcy.5 (AAV7.2) See US20030138772 SEQ ID NO: 18 | AAV16.3 See US20030138772 SEQ ID NO: 105 |
| AAVcy.6 (AAV16.3) See US20030138772 SEQ ID NO: 10 | AAVcy.5 See US20150159173 SEQ ID NO: 8 |
| AAVcy.5 See US20150159173 SEQ ID NO: 24 | AAVCy.5Rl See US20150159173 |
| AAVCy.5R2 See US20150159173 | AAVCy.5R3 See US20150159173 |
| AAVCy.5R4 See US20150159173 | AAVDJ See US20140359799 SEQ ID NO: 3, U.S. Pat. No. |
| | 7,588,772 SEQ ID NO: 2 |
| | AAVDJ See US20140359799 SEQ ID NO: 2, U.S. Pat. No. |
| | 7,588,772 SEQ ID NO: 1 |
| | AAVDJ-8 See U.S. Pat. No. 7,588,772; Grimm et al 2008 |
| AAVDJ-8 See U.S. Pat. No. 7,588,772; Grimm et al 2008 | AAVF5 See US20030138772 SEQ ID NO: 110 |
| AAVH2 See US20030138772 SEQ ID NO: 26 | AAVH6 See US20030138772 SEQ ID NO: 25 |
| AAVhEl. l See U.S. Pat. No. 9,233,131 SEQ ID NO: 44 | AAVhErl.14 See U.S. Pat. No. 9,233,131 SEQ ID NO: 46 |
| AAVhErl.16 See U.S. Pat. No. 9,233,131 SEQ ID NO: 48 | AAVhErl.18 See U.S. Pat. No. 9,233,131 SEQ ID NO: 49 |
| AAVhErl.23 (AAVhEr2.29) See U.S. Pat. No. 9,233,131 | AAVhErl.35 See U.S. Pat. No. 9,233,131 SEQ ID NO: 50 |
| SEQ ID NO: 53 | |
| AAVhErl.36 See U.S. Pat. No. 9,233,131 SEQ ID NO: 52 | AAVhErl.5 See U.S. Pat. No. 9,233,131 SEQ ID NO: 45 |
| AAVhErl.7 See U.S. Pat. No. 9,233,131 SEQ ID NO: 51 | AAVhErl.8 See U.S. Pat. No. 9,233,131 SEQ ID NO: 47 |
| AAVhEr2.16 See U.S. Pat. No. 9,233,131 SEQ ID NO: 55 | AAVhEr2.30 See U.S. Pat. No. 9,233,131 SEQ ID NO: 56 |
| AAVhEr2.31 See U.S. Pat. No. 9,233,131 SEQ ID NO: 58 | AAVhEr2.36 See U.S. Pat. No. 9,233,131 SEQ ID NO: 57 |
| AAVhEr2.4 See U.S. Pat. No. 9,233,131 SEQ ID NO: 54 | AAVhEr3.1 See U.S. Pat. No. 9,233,131 SEQ ID NO: 59 |
| AAVhu.l See US20150315612 SEQ ID NO: 46 | AAVhu.l See US20150315612 SEQ ID NO: 144 |
| AAVhu.lO (AAV16.8) See US20150315612 SEQ ID NO: 56 | AAVhu.lO (AAV16.8) See US20150315612 SEQ ID NO: 156 |
| AAVhu.l l (AAV16.12) See US20150315612 SEQ ID NO: 57 | AAVhu.l l (AAV16.12) See US20150315612 SEQ ID NO: 153 |
| AAVhu.12 See US20150315612 SEQ ID NO: 59 | AAVhu.12 See US20150315612 SEQ ID NO: 154 |
| AAVhu.13 See US20150159173 SEQ ID NO: 16, US20150315612 | |
| SEQ ID NO: 71 | |
| AAVhu.13 See US20150159173 SEQ ID NO: 32, US20150315612 | |
| SEQ ID NO: 129 | |
| AAVhu.136.1 See US20150315612 SEQ ID NO 165 | AAVhu.140.1 See US20150315612 SEQ ID NO 166 |
| AAVhu.140.2 See US20150315612 SEQ ID NO 167 | AAVhu.145.6 See US20150315612 SEQ ID No: 178 |
| AAVhu.15 See US20150315612 SEQ ID NO: 147 | AAVhu.15 (AAV33.4) See US20150315612 SEQ ID NO: 50 |
| AAVhu.156.1 See US20150315612 SEQ ID No: 179 | AAVhu.16 See US20150315612 SEQ ID NO 148 |
| AAVhu.l6 (AAV33.8) See US20150315612 SEQ ID NO 51 | AAVhu.17 See US20150315612 SEQ ID NO 83 |
| AAVhu.l7 (AAV33.12) See US20150315612 SEQ ID NO 4 | AAVhu.172.1 See US20150315612 SEQ ID NO 171 |
| AAVhu.172.2 See US20150315612 SEQ ID NO 172 | AAVhu.173.4 See US20150315612 SEQ ID NO 173 |
| AAVhu.173.8 See US20150315612 SEQ ID NO 175 | AAVhu.18 See US20150315612 SEQ ID NO 52 |
| AAVhu.18 See US20150315612 SEQ ID NO 149 | AAVhu.19 See US20150315612 SEQ ID NO 62 |
| AAVhu.19 See US20150315612 SEQ ID NO 133 | AAVhu.2 See US20150315612 SEQ ID NO 48 |
| AAVhu.2 See US20150315612 SEQ ID NO 143 | AAVhu.20 See US20150315612 SEQ ID NO 63 |
| AAVhu.20 See US20150315612 SEQ ID NO 134 | AAVhu.21 See US20150315612 SEQ ID NO 65 |
| AAVhu.21 See US20150315612 SEQ ID NO 135 | AAVhu.22 See US20150315612 SEQ ID NO 67 |
| AAVhu.22 239 US20150315612 SEQ ID NO 138 | AAVhu.23 See US20150315612 SEQ ID NO 60 |
| AAVhu.23.2 See US20150315612 SEQ ID NO 137 | AAVhu.24 See US20150315612 SEQ ID NO 66 |
| AAVhu.24 See US20150315612 SEQ ID NO 136 | AAVhu.25 See US20150315612 SEQ ID NO 49 |
| AAVhu.25 See US20150315612 SEQ ID NO 146 | AAVhu.26 See US20150159173 SEQ ID NO 17, US20150315612 |
| | SEQ ID NO: 61 |
| | AAVhu.26 See US20150159173 SEQ ID NO: 33, US20150315612 |
| | SEQ |
| | AAVhu.27 See US20150315612 SEQ ID NO: 64 |
| AAVhu.27 See US20150315612 SEQ ID NO: 140 | AAVhu.28 See US20150315612 SEQ ID NO: 68 |
| AAVhu.28 See US20150315612 SEQ ID NO: 130 | AAVhu.29 See US20150315612 SEQ ID NO: 69 |
| AAVhu.29 See US20150159173 SEQ ID NO: 42, US20150315612 | |
| SEQ ID NO: 132 | |
| AAVhu.29 See US20150315612 SEQ ID NO: 225 | AAVhu.29R See US20150159173 |
| AAVhu.3 See US20150315612 SEQ ID NO: 44 | AAVhu.3 See US20150315612 SEQ ID NO: 145 |
| AAVhu.30 See US20150315612 SEQ ID NO: 70 | AAVhu.30 See US20150315612 SEQ ID NO: 131 |
| AAVhu.31 See US20150315612 SEQ ID NO: 1 | AAVhu.31 See US20150315612 SEQ ID NO: 121 |
| AAVhu.32 See US20150315612 SEQ ID NO: 2 | AAVhu.32 See US20150315612 SEQ ID NO: 122 |
| AAVhu.33 See US20150315612 SEQ ID NO: 75 | AAVhu.33 See US20150315612 SEQ ID NO: 124 |
| AAVhu.34 See US20150315612 SEQ ID NO: 72 | AAVhu.34 See US20150315612 SEQ ID NO: 125 |
| AAVhu.35 See US20150315612 SEQ ID NO: 73 | AAVhu.35 See US20150315612 SEQ ID NO: 164 |
| AAVhu.36 See US20150315612 SEQ ID NO: 74 | AAVhu.36 See US20150315612 SEQ ID NO: 126 |
| AAVhu.37 See US20150159173 SEQ ID NO: 34, US20150315612 | |
| SEQ ID NO: 88 | |
| AAVhu.37 (AAV106.1) See US20150315612 SEQ ID NO: 10, | |
| US20150159173 SEQ ID NO: 18 | |
| AAVhu.38 See US20150315612 SEQ ID NO 161 | AAVhu.39 See US20150315612 SEQ ID NO 102 |
| AAVhu.39 (AAVLG-9) See US20150315612 SEQ ID NO 24 | AAVhu.4 See US20150315612 SEQ ID NO 47 |
| AAVhu.4 See US20150315612 SEQ ID NO 141 | AAVhu.40 See US20150315612 SEQ ID NO 87 |
| AAVhu.40 (AAV114.3) See US20150315612 SEQ ID No: 11 | AAVhu.41 See US20150315612 SEQ ID NO: 91 |
| AAVhu.41 (AAV127.2) See US20150315612 SEQ ID NO: 6 | AAVhu.42 See US20150315612 SEQ ID NO: 85 |
| AAVhu.42 (AAV127.5) See US20150315612 SEQ ID NO: 8 | AAVhu.43 See US20150315612 SEQ ID NO: 160 |
| AAVhu.43 See US20150315612 SEQ ID NO: 236 | AAVhu.43 (AAV128.1) See US20150315612 SEQ ID NO: 80 |
| AAVhu.44 See US20150159173 SEQ ID NO: 45, US20150315612 | |
| SEQ ID NO: 158 | |
| AAVhu.44 (AAV128.3) See US20150315612 SEQ ID NO: 81 | AAVhu.44Rl See US20150159173 |
| AAVhu.44R2 See US20150159173 | AAVhu.44R3 See US20150159173 |
| AAVhu.45 See US20150315612 SEQ ID NO: 76 | AAVhu.45 See US20150315612 SEQ ID NO: 127 |
| AAVhu.46 See US20150315612 SEQ ID NO: 82 | AAVhu.46 See US20150315612 SEQ ID NO: 159 |
| AAVhu.46 See US20150315612 SEQ ID NO: 224 | AAVhu.47 See US20150315612 SEQ ID NO: 77 |
| AAVhu.47 See US20150315612 SEQ ID NO: 128 | AAVhu.48 See US20150159173 SEQ ID NO: 38 |
| AAVhu.48 See US20150315612 SEQ ID NO: 157 | AAVhu.48 (AAV130.4) See US20150315612 SEQ ID NO: 78 |
| AAVhu.48Rl See US20150159173 | AAVhu.48R2 See US20150159173 |
| AAVhu.48R3 See US20150159173 | AAVhu.49 See US20150315612 SEQ ID NO 209 |
| AAVhu.49 See US20150315612 SEQ ID NO 189 | AAVhu.5 See US20150315612 SEQ ID NO 45 |
| AAVhu.5 See US20150315612 SEQ ID NO 142 | AAVhu.51 See US20150315612 SEQ ID NO 208 |
| AAVhu.51 See US20150315612 SEQ ID NO 190 | AAVhu.52 See US20150315612 SEQ ID NO 210 |
| AAVhu.52 See US20150315612 SEQ ID NO 191 | AAVhu.53 See US20150159173 SEQ ID NO 19 |
| AAVhu.53 See US20150159173 SEQ ID NO 35 | AAVhu.53 (AAV145.1) See US20150315612 SEQ ID NO 176 |
| AAVhu.54 See US20150315612 SEQ ID NO 188 | AAVhu.54 (AAV145.5) See US20150315612 SEQ ID No: 177 |
| AAVhu.55 See US20150315612 SEQ ID NO 187 | AAVhu.56 See US20150315612 SEQ ID NO 205 |
| AAVhu.56 (AAV145.6) See US20150315612 SEQ ID NO 168 | AAVhu.56 (AAV145.6) See US20150315612 SEQ ID NO 192 |
| AAVhu.57 See US20150315612 SEQ ID NO 206 | AAVhu.57 See US20150315612 SEQ ID NO 169 |
| AAVhu.57 See US20150315612 SEQ ID NO 193 | AAVhu.58 See US20150315612 SEQ ID NO 207 |
| AAVhu.58 See US20150315612 SEQ ID NO 194 | AAVhu.6 (AAV3.1) See US20150315612 SEQ ID NO: 5 |
| AAVhu.6 (AAV3.1) See US20150315612 SEQ ID NO: 84 | AAVhu.60 See US20150315612 SEQ ID NO: 184 |
| AAVhu.60 (AAV161.10) See US20150315612 SEQ ID NO: 170 | AAVhu.61 See US20150315612 SEQ ID NO: 185 |
| AAVhu.61 (AAV161.6) See US20150315612 SEQ ID NO: 174 | AAVhu.63 See US20150315612 SEQ ID NO: 204 |
| AAVhu.63 See US20150315612 SEQ ID NO: 195 | AAVhu.64 See US20150315612 SEQ ID NO: 212 |
| AAVhu.64 See US20150315612 SEQ ID NO: 196 | AAVhu.66 See US20150315612 SEQ ID NO: 197 |
| AAVhu.67 See US20150315612 SEQ ID NO: 215 | AAVhu.67 See US20150315612 SEQ ID NO: 198 |
| AAVhu.7 See US20150315612 SEQ ID NO: 226 | AAVhu.7 See US20150315612 SEQ ID NO: 150 |
| AAVhu.7 (AAV7.3) See US20150315612 SEQ ID NO: 55 | AAVhu.71 See US20150315612 SEQ ID NO: 79 |
| AAVhu.8 See US20150315612 SEQ ID NO: 53 | AAVhu.8 See US20150315612 SEQ ID NO: 12 |
| AAVhu.8 See US20150315612 SEQ ID NO: 151 | AAVhu.9 (AAV3.1) See US20150315612 SEQ ID NO: 58 |
| AAVhu.9 (AAV3.1) See US20150315612 SEQ ID NO: 155 | AAV-LK01 See US20150376607 SEQ ID NO: 2 |
| AAV-LK01 See US20150376607 SEQ ID NO: 29 | AAV-LK02 See US20150376607 SEQ ID NO: 3 |
| AAV-LK02 See US20150376607 SEQ ID NO: 30 | AAV-LK03 See US20150376607 SEQ ID NO: 4 |
| AAV-LK03 See WO2015121501 SEQ ID NO: 12, US20150376607 | |
| SEQ ID NO: 31 | |
| AAV-LK04 See US20150376607 SEQ ID NO: 5 | AAV-LK04 See US20150376607 SEQ ID NO: 32 |
| AAV-LK05 See US20150376607 SEQ ID NO: 6 | AAV-LK05 See US20150376607 SEQ ID NO: 33 |
| AAV-LK06 See US20150376607 SEQ ID NO: 7 | AAV-LK06 See US20150376607 SEQ ID NO: 34 |
| AAV-LK07 See US20150376607 SEQ ID NO: 8 | AAV-LK07 See US20150376607 SEQ ID NO: 35 |
| AAV-LK08 See US20150376607 SEQ ID NO: 9 | AAV-LK08 See US20150376607 SEQ ID NO: 36 |
| AAV-LK09 See US20150376607 SEQ ID NO: 10 | AAV-LK09 See US20150376607 SEQ ID NO: 37 |
| AAV-LK10 See US20150376607 SEQ ID NO: 11 | AAV-LK10 See US20150376607 SEQ ID NO: 38 |
| AAV-LK11 See US20150376607 SEQ ID NO: 12 | AAV-LK11 See US20150376607 SEQ ID NO: 39 |
| AAV-LK12 See US20150376607 SEQ ID NO: 13 | AAV-LK12 See US20150376607 SEQ ID NO: 40 |
| AAV-LK13 See US20150376607 SEQ ID NO: 14 | AAV-LK13 See US20150376607 SEQ ID NO: 41 |
| AAV-LK14 See US20150376607 SEQ ID NO: 15 | AAV-LK14 See US20150376607 SEQ ID NO: 42 |
| AAV-LK15 See US20150376607 SEQ ID NO: 16 | AAV-LK15 See US20150376607 SEQ ID NO: 43 |
| AAV-LK16 See US20150376607 SEQ ID NO: 17 | AAV-LK16 See US20150376607 SEQ ID NO: 44 |
| AAV-LK17 See US20150376607 SEQ ID NO: 18 | AAV-LK17 See US20150376607 SEQ ID NO: 45 |
| AAV-LK18 See US20150376607 SEQ ID NO: 19 | AAV-LK18 See US20150376607 SEQ ID NO: 46 |
| AAV-LK19 See US20150376607 SEQ ID NO: 20 | AAV-LK19 See US20150376607 SEQ ID NO: 47 |
| AAV-PAEC See US20150376607 SEQ ID NO: 1 | AAV-PAEC See US20150376607 SEQ ID NO: 48 |
| AAV-PAEC11 See US20150376607 SEQ ID NO: 26 | AAV-PAEC11 See US20150376607 SEQ ID NO: 54 |
| AAV-PAEC 12 See US20150376607 SEQ ID NO: 27 | AAV-PAEC 12 See US20150376607 SEQ ID NO: 51 |
| AAV-PAEC 13 See US20150376607 SEQ ID NO: 28 | AAV-PAEC 13 See US20150376607 SEQ ID NO: 49 |
| AAV-PAEC2 See US20150376607 SEQ ID NO: 21 | AAV-PAEC2 See US20150376607 SEQ ID NO: 56 |
| AAV-PAEC4 See US20150376607 SEQ ID NO: 22 | AAV-PAEC4 See US20150376607 SEQ ID NO: 55 |
| AAV-PAEC6 See US20150376607 SEQ ID NO: 23 | AAV-PAEC6 See US20150376607 SEQ ID NO: 52 |
| AAV-PAEC7 See US20150376607 SEQ ID NO: 24 | AAV-PAEC7 See US20150376607 SEQ ID NO: 53 |
| AAV-PAEC8 See US20150376607 SEQ ID NO: 25 | AAV-PAEC8 See US20150376607 SEQ ID NO: 50 |
| AAVpi.l See US20150315612 SEQ ID NO: 28 | AAVpi.l See US20150315612 SEQ ID NO: 93 |
| | AAVpi.2 408 US20150315612 SEQ ID NO: 30 |
| AAVpi.2 See US20150315612 SEQ ID NO: 95 | AAVpi.3 See US20150315612 SEQ ID NO: 29 |
| AAVpi.3 See US20150315612 SEQ ID NO: 94 | AAVrh.10 See US20150159173 SEQ ID NO: 9 |
| AAVrh.10 See US20150159173 SEQ ID NO: 25 | AAV44.2 See US20030138772 SEQ ID NO: 59 |
| AAVrh.10 (AAV44.2) See US20030138772 SEQ ID NO: 81 | AAV42.1B See US20030138772 SEQ ID NO: 90 |
| AAVrh.l2 (AAV42.1b) See US20030138772 SEQ ID NO: 30 | AAVrh.13 See US20150159173 SEQ ID NO: 10 |
| AAVrh.13 See US20150159173 SEQ ID NO: 26 | AAVrh.13 See US20150315612 SEQ ID NO: 228 |
| AAVrh.l3R See US20150159173 | AAV42.3A See US20030138772 SEQ ID NO: 87 |
| AAVrh.l4 (AAV42.3a) See US20030138772 SEQ ID NO: 32 | AAV42.5A See US20030138772 SEQ ID NO: 89 |
| AAVrh.l7 (AAV42.5a) See US20030138772 SEQ ID NO: 34 | AAV42.5B See US20030138772 SEQ ID NO: 91 |
| AAVrh.l8 (AAV42.5b) See US20030138772 SEQ ID NO: 29 | AAV42.6B See US20030138772 SEQ ID NO: 112 |
| AAVrh.l9 (AAV42.6b) See US20030138772 SEQ ID NO: 38 | AAVrh.2 See US20150159173 SEQ ID NO: 39 |
| AAVrh.2 See US20150315612 SEQ ID NO: 231 | AAVrh.20 See US20150159173 SEQ ID NO: 1 |
| AAV42.10 See US20030138772 SEQ ID NO: 106 | AAVrh.21 (AAV42.10) See US20030138772 SEQ ID NO: 35 |
| AAV42.11 See US20030138772 SEQ ID NO: 108 | AAVrh.22 (AAV42.11) See US20030138772 SEQ ID NO: 37 |
| AAV42.12 See US20030138772 SEQ ID NO: 113 | AAVrh.23 (AAV42.12) See US20030138772 SEQ ID NO: 58 |
| AAV42.13 See US20030138772 SEQ ID NO: 86 | AAVrh.24 (AAV42.13) See US20030138772 SEQ ID NO: 31 |
| AAV42.15 See US20030138772 SEQ ID NO: 84 | AAVrh.25 (AAV42.15) See US20030138772 SEQ ID NO: 28 |
| AAVrh.2R See US20150159173 | AAVrh.31 (AAV223.1) See US20030138772 SEQ ID NO: 48 |
| AAVC1 See US20030138772 SEQ ID NO: 60 | AAVrh.32 (AAVC1) See 446 US20030138772 SEQ ID NO: 19 |
| AAVrh.32/33 See US20150159173 SEQ ID NO: 2 | AAVrh.51 (AAV2-5) See US20150315612 SEQ ID NO: 104 |
| AAVrh.52 (AAV3-9) See US20150315612 SEQ ID NO: 18 | AAVrh.52 (AAV3-9) See US20150315612 SEQ ID NO: 96 |
| AAVrh.53 See US20150315612 SEQ ID NO: 97 | AAVrh.53 (AAV3-11) See US20150315612 SEQ ID NO: 17 |
| AAVrh.53 (AAV3-11) See US20150315612 SEQ ID NO: 186 | AAVrh.54 See US20150315612 SEQ ID NO: 40 |
| AAVrh.54 See US20150159173 SEQ ID NO: 49, US20150315612 | |
| SEQ ID NO: 116 | |
| AAVrh.55 See US20150315612 SEQ ID NO: 37 | AAVrh.55 (AAV4-19) See US20150315612 SEQ ID NO: 117 |
| AAVrh.56 v US20150315612 SEQ ID NO: 54 | AAVrh.56 See US20150315612 SEQ ID NO: 152 |
| AAVrh.57 See 497 US20150315612 SEQ ID NO: 26 | AAVrh.57 See US20150315612 SEQ ID NO: 105 |
| AAVrh.58 See US20150315612 SEQ ID NO: 27 | AAVrh.58 See US20150159173 SEQ ID NO: 48, US20150315612 |
| | SEQ ID NO: 106 |
| | AAVrh.58 See US20150315612 SEQ ID NO: 232 |
| AAVrh.59 See US20150315612 SEQ ID NO: 42 | AAVrh.59 See US20150315612 SEQ ID NO: 110 |
| AAVrh.60 See US20150315612 SEQ ID NO: 31 | AAVrh.60 See US20150315612 SEQ ID NO: 120 |
| AAVrh.61 See US20150315612 SEQ ID NO: 107 | AAVrh.61 (AAV2-3) See US20150315612 SEQ ID NO: 21 |
| AAVrh.62 (AAV2-15) See US20150315612 SEQ ID No: 33 | AAVrh.62 (AAV2-15) See US20150315612 SEQ ID NO: 114 |
| AAVrh.64 See US20150315612 SEQ ID No: 15 | AAVrh.64 See US20150159173 SEQ ID NO: 43, US20150315612 |
| | SEQ ID NO: 99 |
| | AAVrh.64 See US20150315612 SEQ ID NO: 233 |
| AAVRh.64Rl See US20150159173 | AAVRh.64R2 See US20150159173 |
| AAVrh.65 See US20150315612 SEQ ID NO: 35 | AAVrh.65 See US20150315612 SEQ ID NO: 112 |
| AAVrh.67 See US20150315612 SEQ ID NO: 36 | AAVrh.67 See US20150315612 SEQ ID NO: 230 |
| AAVrh.67 See US20150159173 SEQ ID NO: 47, US20150315612 | |
| SEQ ID NO: 113 | |
| AAVrh.68 See US20150315612 SEQ ID NO: 16 | AAVrh.68 See US20150315612 SEQ ID NO: 100 |
| AAVrh.69 See US20150315612 SEQ ID NO: 39 | AAVrh.69 See US20150315612 SEQ ID NO: 119 |
| AAVrh.70 See US20150315612 SEQ ID NO: 20 | AAVrh.70 See US20150315612 SEQ ID NO: 98 |
| AAVrh.71 See US20150315612 SEQ ID NO: 162 | AAVrh.72 See US20150315612 SEQ ID NO: 9 |
| AAVrh.73 See US20150159173 SEQ ID NO: 5 | AAVrh.74 See US20150159173 SEQ ID NO: 6 |
| AAVrh.8 See US20150159173 SEQ ID NO: 41 | AAVrh.8 See US20150315612 SEQ ID NO: 235 |
| AAVrh.8R See US20150159173, WO2015168666 SEQ ID NO: 9 | AAVrh.8R A586R mutant See WO2015168666 SEQ ID NO: 10 |
| AAVrh.8R R533A mutant See WO2015168666 SEQ ID NO: 11 | BAAV (bovine AAV) See U.S. Pat. No. 9,193,769 SEQ ID NO: 8 |
| BAAV (bovine AAV) See U.S. Pat. No. 9,193,769 SEQ ID NO: 10 | BAAV (bovine AAV) See U.S. Pat. No. 9,193,769 SEQ ID NO: 4 |
| BAAV (bovine AAV) See U.S. Pat. No. 9,193,769 SEQ ID NO: 2 | BAAV (bovine AAV) See U.S. Pat. No. 9,193,769 SEQ ID NO: 6 |
| BAAV (bovine AAV) See U.S. Pat. No. 9,193,769 SEQ ID NO: 1 | BAAV (bovine AAV) See U.S. Pat. No. 9,193,769 SEQ ID NO: 5 |
| BAAV (bovine AAV) See U.S. Pat. No. 9,193,769 SEQ ID NO: 3 | BAAV (bovine AAV) See U.S. Pat. No. 9,193,769 SEQ ID NO: 11 |
| BAAV (bovine AAV) See U.S. Pat. No. 7,427,396 SEQ ID NO: 5 | BAAV (bovine AAV) See U.S. Pat. No. 7,427,396 SEQ ID NO: 6 |
| BAAV (bovine AAV) See U.S. Pat. No. 9,193,769 SEQ ID NO: 7 | BAAV (bovine AAV) See U.S. Pat. No. 9,193,769 SEQ ID NO: 9 |
| BNP61 AAV See US20150238550 SEQ ID NO: 1 | BNP61 AAV See US20150238550 SEQ ID NO: 2 |
| BNP62 AAV See US20150238550 SEQ ID NO: 3 | BNP63 AAV See US20150238550 SEQ ID NO: 4 |
| caprine AAV See U.S. Pat. No. 7,427,396 SEQ ID NO: 3 | caprine AAV See U.S. Pat. No. 7,427,396 SEQ ID NO: 4 |
| true type AAV (ttAAV) See WO2015121501 SEQ ID NO: 2 | AAAV (Avian AAV) See U.S. Pat. No. 9,238,800 SEQ ID NO: 12 |
| AAAV (Avian AAV) See U.S. Pat. No. 9,238,800 SEQ ID NO: 2 | AAAV (Avian AAV) See U.S. Pat. No. 9,238,800 SEQ ID NO: 6 |
| AAAV (Avian AAV) See U.S. Pat. No. 9,238,800 SEQ ID NO: 4 | AAAV (Avian AAV) See U.S. Pat. No. 9,238,800 SEQ ID NO: 8 |
| AAAV (Avian AAV) See U.S. Pat. No. 9,238,800 SEQ ID NO: 14 | AAAV (Avian AAV) See U.S. Pat. No. 9,238,800 SEQ ID NO: 10 |
| AAAV (Avian AAV) See U.S. Pat. No. 9,238,800 SEQ ID NO: 15 | AAAV (Avian AAV) See U.S. Pat. No. 9,238,800 SEQ ID NO: 5 |
| AAAV (Avian AAV) See U.S. Pat. No. 9,238,800 SEQ ID NO: 9 | AAAV (Avian AAV) See U.S. Pat. No. 9,238,800 SEQ ID NO: 3 |
| AAAV (Avian AAV) See U.S. Pat. No. 9,238,800 SEQ ID NO: 7 | AAAV (Avian AAV) See U.S. Pat. No. 9,238,800 SEQ ID NO: 11 |
| AAAV (Avian AAV) See U.S. Pat. No. 9,238,800 SEQ ID NO: 13 | AAAV (Avian AAV) See U.S. Pat. No. 9,238,800 SEQ ID NO: 1 |
| AAV Shuffle 100-1 See US20160017295 SEQ ID NO: 23 | AAV Shuffle 100-1 See US20160017295 SEQ ID NO: 11 |
| AAV Shuffle 100-2 See US20160017295 SEQ ID NO: 37 | AAV Shuffle 100-2 See US20160017295 SEQ ID NO: 29 |
| AAV Shuffle 100-3 See US20160017295 SEQ ID NO: 24 | AAV Shuffle 100-3 See US20160017295 SEQ ID NO: 12 |
| AAV Shuffle 100-7 See US20160017295 SEQ ID NO: 25 | AAV Shuffle 100-7 See US20160017295 SEQ ID NO: 13 |
| AAV Shuffle 10-2 See US20160017295 SEQ ID NO: 34 | AAV Shuffle 10-2 See US20160017295 SEQ ID NO: 26 |
| AAV Shuffle 10-6 See US20160017295 SEQ ID NO: 35 | AAV Shuffle 10-6 See US20160017295 SEQ ID NO: 27 |
| AAV Shuffle 10-8 See US20160017295 SEQ ID NO: 36 | AAV Shuffle 10-8 See US20160017295 SEQ ID NO: 28 |
| AAV SM 100-10 See US20160017295 SEQ ID NO: 41 | AAV SM 100-10 See US20160017295 SEQ ID NO: 33 |
| AAV SM 100-3 See US20160017295 SEQ ID NO: 40 | AAV SM 100-3 See US20160017295 SEQ ID NO: 32 |
| AAV SM 10-1 See US20160017295 SEQ ID NO: 38 | AAV SM 10-1 See US20160017295 SEQ ID NO: 30 |
| AAV SM 10-2 See US20160017295 SEQ ID NO: 10 | AAV SM 10-2 See US20160017295 SEQ ID NO: 22 |
| AAV SM 10-8 See US20160017295 SEQ ID NO: 39 | AAV SM 10-8 See US20160017295 SEQ ID NO: 31 |
| AAV CBr-7.1 See WO2016065001 SEQ ID NO: 4 | AAV CBr-7.1 See WO2016065001 SEQ ID NO: 54 |
| AAV CBr-7.10 See WO2016065001 SEQ ID NO: 11 | AAV CBr-7.10 See WO2016065001 SEQ ID NO: 61 |
| AAV CBr-7.2 See WO2016065001 SEQ ID NO: 5 | AAV CBr-7.2 See WO2016065001 SEQ ID NO: 55 |
| AAV CBr-7.3 See WO2016065001 SEQ ID NO: 6 | AAV CBr-7.3 See WO2016065001 SEQ ID NO: 56 |
| AAV CBr-7.4 See WO2016065001 SEQ ID NO: 7 | AAV CBr-7.4 See WO2016065001 SEQ ID NO: 57 |
| AAV CBr-7.5 See WO2016065001 SEQ ID NO: 8 | AAV CHt-6.6 See WO2016065001 SEQ ID NO: 35 |
| AAV CHt-6.6 See WO2016065001 SEQ ID NO: 85 | AAV CHt-6.7 See WO2016065001 SEQ ID NO: 36 |
| AAV CHt-6.7 See WO2016065001 SEQ ID NO: 86 | AAV CHt-6.8 See WO2016065001 SEQ ID NO: 37 |
| AAV CHt-6.8 See WO2016065001 SEQ ID NO: 87 | AAV CHt-Pl See WO2016065001 SEQ ID NO: 29 |
| AAV CHt-Pl See WO2016065001 SEQ ID NO: 79 | AAV CHt-P2 See WO2016065001 SEQ ID NO: 1 |
| AAV CHt-P2 See WO2016065001 SEQ ID NO: 51 | AAV CHt-P5 See WO2016065001 SEQ ID NO: 2 |
| AAV CHt-P5 See WO2016065001 SEQ ID NO: 52 | AAV CHt-P6 See WO2016065001 SEQ ID NO: 30 |
| AAV CHt-P6 See WO2016065001 SEQ ID NO: 80 | AAV CHt-P8 See WO2016065001 SEQ ID NO: 31 |
| AAV CHt-P8 See WO2016065001 SEQ ID NO: 81 | AAV CHt-P9 See WO2016065001 SEQ ID NO: 3 |
| AAV CHt-P9 See WO2016065001 SEQ ID NO: 53 | AAV CKd-1 See U.S. Pat. No. 8,734,809 SEQ ID NO 57 |
| AAV CKd-1 See U.S. Pat. No. 8,734,809 SEQ ID NO 131 | AAV CKd-10 See U.S. Pat. No. 8,734,809 SEQ ID NO 58 |
| AAV CKd-10 See U.S. Pat. No. 8,734,809 SEQ ID NO 132 | AAV CKd-2 See U.S. Pat. No. 8,734,809 SEQ ID NO 59 |
| AAV CKd-2 See U.S. Pat. No. 8,734,809 SEQ ID NO 133 | AAV CKd-3 See U.S. Pat. No. 8,734,809 SEQ ID NO 60 |
| AAV CKd-3 See U.S. Pat. No. 8,734,809 SEQ ID NO 134 | AAV CKd-4 See U.S. Pat. No. 8,734,809 SEQ ID NO 61 |
| AAV CKd-4 See U.S. Pat. No. 8,734,809 SEQ ID NO 135 | AAV CKd-6 See U.S. Pat. No. 8,734,809 SEQ ID NO 62 |
| AAV CKd-6 See U.S. Pat. No. 8,734,809 SEQ ID NO 136 | AAV CKd-7 See U.S. Pat. No. 8,734,809 SEQ ID NO 63 |
| AAV CKd-7 See U.S. Pat. No. 8,734,809 SEQ ID NO 137 | AAV CKd-8 See U.S. Pat. No. 8,734,809 SEQ ID NO 64 |
| AAV CKd-8 See U.S. Pat. No. 8,734,809 SEQ ID NO 138 | AAV CKd-B 1 See U.S. Pat. No. 8,734,809 SEQ ID NO 73 |
| AAV CKd-B 1 See U.S. Pat. No. 8,734,809 SEQ ID NO 147 | AAV CKd-B2 See U.S. Pat. No. 8,734,809 SEQ ID NO 74 |
| AAV CKd-B2 See U.S. Pat. No. 8,734,809 SEQ ID NO 148 | AAV CKd-B3 See U.S. Pat. No. 8,734,809 SEQ ID NO 75 |
| AAV CKd-B3 See U.S. Pat. No. 8,734,809 | AAV CKd-B3 See U.S. Pat. No. 8,734,809 SEQ ID NO 149 |
| AAV CLv-1 See U.S. Pat. No. 8,734,809 SEQ ID NO: 65 | AAV CLv-1 See U.S. Pat. No. 8,734,809 SEQ ID NO: 139 |
| AAV CLvl-1 See U.S. Pat. No. 8,734,809 SEQ ID NO: 171 | AAV Civ 1-10 See U.S. Pat. No. 8,734,809 SEQ ID NO: 178 |
| AAV CLvl-2 See U.S. Pat. No. 8,734,809 SEQ ID NO: 172 | AAV CLv-12 See U.S. Pat. No. 8,734,809 SEQ ID NO: 66 |
| AAV CLv-12 See U.S. Pat. No. 8,734,809 SEQ ID NO: 140 | AAV CLvl-3 See U.S. Pat. No. 8,734,809 SEQ ID NO: 173 |
| AAV CLv-13 See U.S. Pat. No. 8,734,809 SEQ ID NO: 67 | AAV CLv-13 See U.S. Pat. No. 8,734,809 SEQ ID NO: 141 |
| AAV CLvl-4 See U.S. Pat. No. 8,734,809 SEQ ID NO: 174 | AAV Civ 1-7 See U.S. Pat. No. 8,734,809 SEQ ID NO: 175 |
| AAV Civ 1-8 See U.S. Pat. No. 8,734,809 SEQ ID NO: 176 | AAV Civ 1-9 See U.S. Pat. No. 8,734,809 SEQ ID NO: 177 |
| AAV CLv-2 See U.S. Pat. No. 8,734,809 SEQ ID NO: 68 | AAV CLv-2 See U.S. Pat. No. 8,734,809 SEQ ID NO: 142 |
| AAV CLv-3 See U.S. Pat. No. 8,734,809 SEQ ID NO: 69 | AAV CLv-3 See U.S. Pat. No. 8,734,809 SEQ ID NO: 143 |
| AAV CLv-4 See U.S. Pat. No. 8,734,809 SEQ ID NO: 70 | AAV CLv-4 See U.S. Pat. No. 8,734,809 SEQ ID NO: 144 |
| AAV CLv-6 See U.S. Pat. No. 8,734,809 SEQ ID NO: 71 | AAV CLv-6 See U.S. Pat. No. 8,734,809 SEQ ID NO: 145 |
| AAV CLv-8 See U.S. Pat. No. 8,734,809 SEQ ID NO: 72 | AAV CLv-8 See U.S. Pat. No. 8,734,809 SEQ ID NO: 146 |
| AAV CLv-Dl See U.S. Pat. No. 8,734,809 SEQ ID NO: 22 | AAV CLv-Dl See U.S. Pat. No. 8,734,809 SEQ ID NO: 96 |
| AAV CLv-D2 See U.S. Pat. No. 8,734,809 SEQ ID NO: 23 | AAV CLv-D2 See U.S. Pat. No. 8,734,809 SEQ ID NO: 97 |
| AAV CLv-D3 See U.S. Pat. No. 8,734,809 SEQ ID NO: 24 | AAV CLv-D3 See U.S. Pat. No. 8,734,809 SEQ ID NO: 98 |
| AAV CLv-D4 See U.S. Pat. No. 8,734,809 SEQ ID NO: 25 | AAV CLv-D4 See U.S. Pat. No. 8,734,809 SEQ ID NO: 99 |
| AAV CLv-D5 See U.S. Pat. No. 8,734,809 SEQ ID NO: 26 | AAV CLv-D5 See U.S. Pat. No. 8,734,809 SEQ ID NO: 100 |
| AAV CLv-D6 See U.S. Pat. No. 8,734,809 SEQ ID NO: 27 | AAV CLv-D6 See U.S. Pat. No. 8,734,809 SEQ ID NO: 101 |
| AAV CLv-D7 See U.S. Pat. No. 8,734,809 SEQ ID NO: 28 | AAV CLv-D7 See U.S. Pat. No. 8,734,809 SEQ ID NO: 102 |
| AAV CLv-D8 See U.S. Pat. No. 8,734,809 SEQ ID NO: 29 | AAV CLv-D8 See U.S. Pat. No. 8,734,809 SEQ ID NO: 103 |
| | AAV CLv-Kl 762 WO2016065001 SEQ ID NO: 18 |
| AAV CLv-Kl See WO2016065001 SEQ ID NO: 68 | AAV CLv-K3 See WO2016065001 SEQ ID NO: 19 |
| AAV CLv-K3 See WO2016065001 SEQ ID NO: 69 | AAV CLv-K6 See WO2016065001 SEQ ID NO: 20 |
| AAV CLv-K6 See WO2016065001 SEQ ID NO: 70 | AAV CLv-L4 See WO2016065001 SEQ ID NO: 15 |
| AAV CLv-L4 See WO2016065001 SEQ ID NO: 65 | AAV CLv-L5 See WO2016065001 SEQ ID NO: 16 |
| AAV CLv-L5 See WO2016065001 SEQ ID NO: 66 | AAV CLv-L6 See WO2016065001 SEQ ID NO: 17 |
| AAV CLv-L6 See WO2016065001 SEQ ID NO: 67 | AAV CLv-Ml See WO2016065001 SEQ ID NO: 21 |
| AAV CLv-Ml See WO2016065001 SEQ ID NO: 71 | AAV CLv-Mll See WO2016065001 SEQ ID NO: 22 |
| AAV CLv-Ml 1 See WO2016065001 SEQ ID NO: 72 | AAV CLv-M2 See WO2016065001 SEQ ID NO: 23 |
| AAV CLv-M2 See WO2016065001 SEQ ID NO: 73 | AAV CLv-M5 See WO2016065001 SEQ ID NO: 24 |
| AAV CLv-M5 See WO2016065001 SEQ ID NO: 74 | AAV CLv-M6 See WO2016065001 SEQ ID NO: 25 |
| AAV CLv-M6 See WO2016065001 SEQ ID NO: 75 | AAV CLv-M7 See WO2016065001 SEQ ID NO: 26 |
| AAV CLv-M7 See WO2016065001 SEQ ID NO: 76 | AAV CLv-M8 See WO2016065001 SEQ ID NO: 27 |
| AAV CLv-M8 See WO2016065001 SEQ ID NO: 77 | AAV CLv-M9 See WO2016065001 SEQ ID NO: 28 |
| AAV CLv-M9 See WO2016065001 SEQ ID NO: 78 | AAV CLv-Rl See U.S. Pat. No. 8,734,809 SEQ ID NO 30 |
| AAV CLv-Rl See U.S. Pat. No. 8,734,809 SEQ ID NO 104 | AAV CLv-R2 See U.S. Pat. No. 8,734,809 SEQ ID NO 31 |
| AAV CLv-R2 See U.S. Pat. No. 8,734,809 SEQ ID NO 105 | AAV CLv-R3 See U.S. Pat. No. 8,734,809 SEQ ID NO 32 |
| AAV CLv-R3 See U.S. Pat. No. 8,734,809 SEQ ID NO 106 | AAV CLv-R4 See U.S. Pat. No. 8,734,809 SEQ ID NO 33 |
| AAV CLv-R4 See U.S. Pat. No. 8,734,809 SEQ ID NO 107 | AAV CLv-R5 See U.S. Pat. No. 8,734,809 SEQ ID NO 34 |
| AAV CLv-R5 See U.S. Pat. No. 8,734,809 SEQ ID NO 108 | AAV CLv-R6 See U.S. Pat. No. 8,734,809 SEQ ID NO 35 |
| AAV CLv-R6 See U.S. Pat. No. 8,734,809 SEQ ID NO 109 | AAV CLv-R7 See U.S. Pat. No. 8,734,809 SEQ ID NO 110 |
| AAV CLv-R7 802 U.S. Pat. No. 8,734,809 SEQ ID NO 36 | |
| AAV CLv-R8 See U.S. Pat. No. 8,734,809 SEQ ID NO 37 | AAV CLv-R8 See U.S. Pat. No. 8,734,809 SEQ ID NO 111 |
| AAV CLv-R9 See U.S. Pat. No. 8,734,809 SEQ ID NO 38 | AAV CLv-R9 See U.S. Pat. No. 8,734,809 SEQ ID NO 112 |
| AAV CSp-1 See U.S. Pat. No. 8,734,809 SEQ ID NO 45 | AAV CSp-1 See U.S. Pat. No. 8,734,809 SEQ ID NO 119 |
| AAV CSp-10 See U.S. Pat. No. 8,734,809 SEQ ID NO 46 | AAV CSp-10 See U.S. Pat. No. 8,734,809 SEQ ID NO 120 |
| AAV CSp-11 See U.S. Pat. No. 8,734,809 SEQ ID NO 47 | AAV CSp-11 See U.S. Pat. No. 8,734,809 SEQ ID NO 121 |
| AAV CSp-2 See U.S. Pat. No. 8,734,809 SEQ ID NO 48 | AAV CSp-2 See U.S. Pat. No. 8,734,809 SEQ ID NO 122 |
| AAV CSp-3 See U.S. Pat. No. 8,734,809 SEQ ID NO 49 | AAV CSp-3 See U.S. Pat. No. 8,734,809 SEQ ID NO 123 |
| AAV CSp-4 See U.S. Pat. No. 8,734,809 SEQ ID NO 50 | AAV CSp-4 See U.S. Pat. No. 8,734,809 SEQ ID NO 124 |
| AAV CSp-6 See U.S. Pat. No. 8,734,809 SEQ ID NO 51 | AAV CSp-6 See U.S. Pat. No. 8,734,809 SEQ ID NO 125 |
| AAV CSp-7 See U.S. Pat. No. 8,734,809 SEQ ID NO 52 | AAV CSp-7 See U.S. Pat. No. 8,734,809 SEQ ID NO 126 |
| AAV CSp-8 See U.S. Pat. No. 8,734,809 SEQ ID NO 53 | AAV CSp-8 See U.S. Pat. No. 8,734,809 SEQ ID NO 127 |
| AAV CSp-8.10 See WO2016065001 SEQ ID NO: 38 | AAV CSp-8.10 See WO2016065001 SEQ ID NO: 88 |
| AAV CSp-8.2 See WO2016065001 SEQ ID NO: 39 | AAV CSp-8.2 See WO2016065001 SEQ ID NO: 89 |
| AAV CSp-8.4 See WO2016065001 SEQ ID NO: 40 | AAV CSp-8.4 See WO2016065001 SEQ ID NO: 90 |
| AAV CSp-8.5 See WO2016065001 SEQ ID NO: 41 | AAV CSp-8.5 See WO2016065001 SEQ ID NO: 91 |
| AAV CSp-8.6 See WO2016065001 SEQ ID NO: 42 | AAV CSp-8.6 See WO2016065001 SEQ ID NO: 92 |
| AAV CSp-8.7 See WO2016065001 SEQ ID NO: 43 | AAV CSp-8.7 See WO2016065001 SEQ ID NO: 93 |
| AAV CSp-8.8 See WO2016065001 SEQ ID NO: 44 | AAV CSp-8.8 See WO2016065001 SEQ ID NO: 94 |
| AAV CSp-8.9 See WO2016065001 SEQ ID NO: 45 | AAV CSp-8.9 See WO2016065001 SEQ ID NO: 95 |
| AAV CSp-9 842 U.S. Pat. No. 8,734,809 SEQ ID NO: 54 | AAV CSp-9 See U.S. Pat. No. 8,734,809 SEQ ID NO: 128 |
| AAV.hu.48R3 See U.S. Pat. No. 8,734,809 SEQ ID NO: 183 | AAV.VR-355 See U.S. Pat. No. 8,734,809 SEQ ID NO: 181 |
| AAV3B See WO2016065001 SEQ ID NO: 48 | AAV3B See WO2016065001 SEQ ID NO: 98 |
| AAV4 See WO2016065001 SEQ ID NO: 49 | AAV4 See WO2016065001 SEQ ID NO: 99 |
| AAV5 See WO2016065001 SEQ ID NO: 50 | AAV5 See WO2016065001 SEQ ID NO: 100 |
| AAVF1/HSC1 See WO2016049230 SEQ ID NO: 20 | AAVF1/HSC1 See WO2016049230 SEQ ID NO: 2 |
| AAVF11/HSC11 See WO2016049230 SEQ ID NO: 26 | AAVF11/HSC11 See WO2016049230 SEQ ID NO: 4 |
| AAVF12/HSC12 See WO2016049230 SEQ ID NO: 30 | AAVF12/HSC12 See WO2016049230 SEQ ID NO: 12 |
| AAVF13/HSC13 See WO2016049230 SEQ ID NO: 31 | AAVF13/HSC13 See WO2016049230 SEQ ID NO: 14 |
| AAVF14/HSC14 See WO2016049230 SEQ ID NO: 32 | AAVF14/HSC14 See WO2016049230 SEQ ID NO: 15 |
| AAVF15/HSC15 See WO2016049230 SEQ ID NO: 33 | AAVF15/HSC15 See WO2016049230 SEQ ID NO: 16 |
| AAVF16/HSC16 See WO2016049230 SEQ ID NO: 34 | AAVF16/HSC16 See WO2016049230 SEQ ID NO: 17 |
| AAVF17/HSC17 See WO2016049230 SEQ ID NO: 35 | AAVF17/HSC17 See WO2016049230 SEQ ID NO: 13 |
| AAVF2/HSC2 See WO2016049230 SEQ ID NO: 21 | AAVF2/HSC2 See WO2016049230 SEQ ID NO: 3 |
| AAVF3/HSC3 See WO2016049230 SEQ ID NO: 22 | AAVF3/HSC3 See WO2016049230 SEQ ID NO: 5 |
| AAVF4/HSC4 See WO2016049230 SEQ ID NO: 23 | AAVF4/HSC4 See WO2016049230 SEQ ID NO: 6 |
| AAVF5/HSC5 See WO2016049230 SEQ ID NO: 25 | AAVF5/HSC5 See WO2016049230 SEQ ID NO: 11 |
| AAVF6/HSC6 See WO2016049230 SEQ ID NO: 24 | AAVF6/HSC6 See WO2016049230 SEQ ID NO: 7 |
| AAVF7/HSC7 See WO2016049230 SEQ ID NO: 27 | AAVF7/HSC7 See WO2016049230 SEQ ID NO: 8 |
| AAVF8/HSC8 See WO2016049230 SEQ ID NO: 28 | AAVF8/HSC8 See WO2016049230 SEQ ID NO: 9 |
| | AAVF9/HSC9 882 WO2016049230 SEQ ID NO: 29 |
| AAVF9/HSC9 See WO2016049230 SEQ ID NO: 10 | |

The genomic sequences of the various serotypes of AAV and the autonomous parvoviruses, as well as the sequences of the terminal repeats (TRs), Rep proteins, and capsid subunits are known in the art. Such sequences may be found in the literature or in public databases such as GenBank. See, e.g., GenBank Accession Numbers NC_002077, NC_001401, NC_001729, NC_001863, NC_001829, NC_001862, NC 000883, NC_001701, NC_001510, AF063497, U89790, AF043303, AF028705, AF028704, J02275, J01901, J02275, X01457, AF288061, AH009962, AY028226, AY028223, NC_001358, NC_001540, AF513851, AF513852; the disclosures of which are incorporated herein in their entirety. See also, e.g., Srivistava et al., (1983) J. Virology 45:555; Chiorini et al., (1998) J. Virology 71:6823; Chiorini et al., (1999) J. Virology 73:1309; Bantel-Schaal et al., (1999) J. Virology 73:939; Xiao et al., (1999) J. Virology 73:3994; Muramatsu et al., (1996) Virology 221:208; Shade et al., (1986) J. Virol. 58:921; Gao et al., (2002) Proc. Nat. Acad. Sci. USA 99:11854; international patent publications WO 00/28061, WO 99/61601, WO 98/11244; U.S. Pat. No. 6,156,303; the disclosures of which are incorporated herein in their entirety. An early description of the AAV1, AAV2 and AAV3 terminal repeat sequences is provided by Xiao, X., (1996), “Characterization of Adeno-associated virus (AAV) DNA replication and integration,” Ph.D. Dissertation, University of Pittsburgh, Pittsburgh, Pa. (incorporated herein it its entirety).

The parvovirus AAV particles of the invention may be “hybrid” parvovirus or AAV particles in which the viral terminal repeats and viral capsid are from different parvoviruses or AAV, respectively. Hybrid parvoviruses are described in more detail in international patent publication WO 00/28004; Chao et al., (2000) Molecular Therapy 2:619; and Chao et al., (2001) Mol. Ther. 4:217 (the disclosures of which are incorporated herein in their entireties). In representative embodiments, the viral terminal repeats and capsid are from different serotypes of AAV (i.e., a “hybrid AAV particle”).

The parvovirus or AAV capsid may further be a “chimeric” capsid (e.g., containing sequences from different parvoviruses, preferably different AAV serotypes) or a “targeted” capsid (e.g., having a directed tropism) as described in international patent publication WO 00/28004.

Further, the parvovirus or AAV vector may be a duplexed parvovirus particle or duplexed AAV particle as described in international patent publication WO 01/92551.

Adeno-associated viruses (AAV) have been employed as nucleic acid delivery vectors. For a review, see Muzyczka et al. Curr. Topics in Micro. and Immunol. (1992) 158:97-129). AAV are parvoviruses and have small icosahedral virions, 18-26 nanometers in diameter and contain a single stranded genomic DNA molecule 4-5 kilobases in size. The viruses contain either the sense or antisense strand of the DNA molecule and either strand is incorporated into the virion. Two open reading frames encode a series of Rep and Cap polypeptides. Rep polypeptides (Rep50, Rep52, Rep68 and Rep78) are involved in replication, rescue and integration of the AAV genome, although significant activity can be observed in the absence of all four Rep polypeptides. The Cap proteins (VP1, VP2, VP3) form the virion capsid. Flanking the rep and cap open reading frames at the 5′ and 3′ ends of the genome are 145 basepair inverted terminal repeats (ITRs), the first 125 basepairs of which are capable of forming Y- or T-shaped duplex structures. It has been shown that the ITRs represent the minimal cis sequences required for replication, rescue, packaging and integration of the AAV genome. All other viral sequences are dispensable and may be supplied in trans (Muzyczka, (1992) Curr. Topics Microbiol. Immunol. 158:97).

AAV are among the few viruses that can integrate their DNA into non-dividing cells, and exhibit a high frequency of stable integration into human chromosome 19 (see, for example, Flotte et al. (1992) Am. J. Respir. Cell. Mol. Biol. 7:349-356; Samulski et al., (1989) J Virol. 63:3822-3828; and McLaughlin et al., (1989) J. Virol. 62:1963-1973). A variety of nucleic acids have been introduced into different cell types using AAV vectors (see, for example, Hermonat et al., (1984) Proc. Nat. Acad. Sci. USA 81:6466-6470; Tratschin et al., (1985) Mol. Cell. Biol. 4:2072-2081; Wondisford et al., (1988) Mol. Endocrinol. 2:32-39; Tratschin et al., (1984) J. Virol. 51:611-619; and Flotte et al., (1993) J. Biol. Chem. 268:3781-3790).

Generally, a rAAV vector genome will only retain the terminal repeat (TR) sequence(s) so as to maximize the size of the transgene that can be efficiently packaged by the vector. The structural and non-structural protein coding sequences may be provided in trans (e.g., from a vector, such as a plasmid, or by stably integrating the sequences into a packaging cell). Typically, the rAAV vector genome comprises at least one AAV terminal repeat, more typically two AAV terminal repeats, which generally will be at the 5′ and 3′ ends of the heterologous nucleotide sequence(s).

Table 3 describe exemplary chimeric or variant capsid proteins that can be used as the AAV capsid in the rAAV vector described herein, or with any combination with wild type capsid proteins and/or other chimeric or variant capsid proteins now known or later identified and each is incorporated herein. In some embodiments, the rAAV vector encompassed for use is a chimeric vector, e.g., as disclosed in 9,012,224 and U.S. Pat. No. 7,892,809, which are incorporated herein in their entirety by reference.

In some embodiments, the rAAV vector is a haploid rAAV vector, as disclosed in PCT/US18/22725, or polyploid rAAV vector, e.g., as disclosed in PCT/US2018/044632 filed on Jul. 31, 2018 and in U.S. application Ser. No. 16/151,110, each of which are incorporated herein in their entirety by reference. In some embodiments, the rAAV vector is a rAAV3 vector, as disclosed in 9,012,224 and WO 2017/106236 which are incorporated herein in their entirety by reference.

TABLE 3
Exemplary chimeric or variant capsid proteins that can be
used as the AAV capsid in the rAAV vector described herein.

| Chimeric or | | Chimeric or | |
|---|---|---|---|
| variant capsid | reference | variant capsid | Reference |
| LK03 and others | Lisowski et al. [REF 1] | AAV-leukemia targeting | Michelfelder S [REF 30] |
| LK0-19 | | | |
| AAV-DJ | Grimm et al., [REF 2] | AAV-tumor targeting | Muller O J, et al., [REF 31] |
| Olig001 | Powell S K et al., [REF 3] | AAV-tumor targeting | Grifman M et al., [REF 32] |
| rAAV2-retro | Tervo D et al., [REF 4] | AAV2 efficient targeting | Girod et al., [REF 33] |
| AAV-LiC | Marsic D et al., [REF 5] | AAVpo2.1, -po4, -poS, | Bello A, et al., [REF 34] |
| | | and -po6). | |
| (AAV-Keral, AAV- | Sallach et al., [REF 6] | AAV rh and AAV Hu | Gao G, et al., [REF 35] |
| Kera2, and AAV- | | | |
| Kera3) | | | |
| AAV 7m8 | Dalkara et al., [REF 7] | AAV-Go.1 | Arbetman A E et al., [REF 36] |
| (AAV1.9 | Asuri P et al., [REF 8] | AAV-mo.1 | Lochrie M A et al., [REF 37] |
| AAV r3.45 | Jang J H et al., [REF 9] | BAAV | Schmidt M, et al., [REF 38] |
| AAV clone 32 and | Gray S J, et al., [REF 10] | AAAV | Bossis I et al., [REF 39] |
| 83) | | | |
| AAV-U87R7-C5 | Maguire et al., [REF 11] | AAV variants | Chen C L et al., [REF 40] |
| AAV ShH13, AAV | Koerber et al., [REF 12] | AAV8 K137R | Sen D et al., [REF 41] |
| ShH19, AAVLl-12 | | | |
| AAV HAE-1, AAV | Li W et al., [REF 13] | AAV2 Y | Li B, et al., [REF 42] |
| HAE-2 | | | |
| AAV variant ShH10 | Klimczak et al., [REF 14] | AAV2 | Gabriel N et al., [REF 43] |
| AAV2.5T | Excoffon et al., [REF 15] | AAV Anc80L65 | Zinn E, et al., [REF 44] |
| AAV LS1-4, AAV | Sellner L et al., [REF 16] | AAV2G9 | Shen S et al., [REF 45] |
| Lsm | | | |
| AAV1289 | Li W, et al., [REF 17] | AAV2 265 insertion- | Li C, et al., [REF 46] |
| | | AAV2/265D | |
| AAVHSC 1-17 | Charbel Issa P et al., [REF 18] | AAV2.5 | Bowles D E, et al., [REF 47] |
| AAV2 Rec 1-4 | Huang W, et al., [REF 19] | AAV3 SASTG | Messina E L et al., [REF 48] |
| | | | and [REF 55]. (Piacentio et al., |
| | | | (Hum Gen Ther, 2012, 23: 635-646)) |
| AAV8BP2 | Cronin T, et al., [REF 20] | AAV2i8 | Asokan A et al., [REF 49] |
| AAV-B1 | Choudhury S R, et al., [REF 21] | AAV8G9 | Vance M, et al., [REF 50] |
| AAV-PHP.B | Deverman B E, et al., [REF 22] | AAV2 tyrosine | Zhong L et al., [REF 51] |
| | | mutants AAV2 Y-F | |
| AAV9.45, AAV9.61, | Pulicherla N[REF 23], et al., | AAV8 Y-F and AAV9 | Petrs-Silva H et al., [REF 52] |
| AAV9.47 | | Y-F | |
| AAVM41 | Yang L et al., [REF 24] | AAV6 Y-F | Qiao C et al., [REF 53] |
| AAV2 displayed | Korbelin J et al. [REF 25], | (AAV6.2) PCT | Carlon M, et al., [REF 54] |
| peptides) | | Publication No. | |
| | | WO2013158879Al | |
| | | (lysine mutants) | |
| AAV2-GMN | Geoghegan J C [REF 26] | | |
| AAV9-peptide | Varadi K, et al., [REF 27] | | |
| displayed | | | |
| AAV8 and AAV9 | Michelfelder et al., [REF 28] | | |
| peptide displayed | | | |
| AAV2-muscle | Yu C Y et al., [REF 29] | | |
| targeting peptide | | | |

In one embodiment, the rAAV vector as disclosed herein comprises a capsid protein, associated with any of the following biological sequence files listed in the file wrappers of USPTO issued patents and published applications, which describe chimeric or variant capsid proteins that can be incorporated into the AAV capsid of this invention in any combination with wild type capsid proteins and/or other chimeric or variant capsid proteins now known or later identified (for demonstrative purposes, 11486254 corresponds to U.S. patent application Ser. No. 11/486,254 and the other biological sequence files are to be read in a similar manner): 11486254.raw, 11932017.raw, 12172121.raw, 12302206.raw, 12308959.raw, 12679144.raw, 13036343.raw, 13121532.raw, 13172915.raw, 13583920.raw, 13668120.raw, 13673351.raw, 13679684.raw, 14006954.raw, 14149953.raw, 14192101.raw, 14194538.raw, 14225821.raw, 14468108.raw, 14516544.raw, 14603469.raw, 14680836.raw, 14695644.raw, 14878703.raw, 14956934.raw, 15191357.raw, 15284164.raw, 15368570.raw, 15371188.raw, 15493744.raw, 15503120.raw, 15660906.raw, and 15675677.raw. In an embodiment, the AAV capsid proteins and virus capsids of this invention can be chimeric in that they can comprise all or a portion of a capsid subunit from another virus, optionally another parvovirus or AAV, e.g., as described in international patent publication WO 00/28004, which is incorporated by reference.

In some embodiments, an rAAV vector genome is single stranded or a monomeric duplex as described in U.S. Pat. No. 8,784,799, which is incorporated herein.

As a further embodiment, the AAV capsid proteins and virus capsids of this invention can be polyploid (also referred to as haploid) in that they can comprise different combinations of VP1, VP2 and VP3 AAV serotypes in a single AAV capsid as described in PCT/US18/22725, which is incorporated by reference.

In an embodiment, an rAAV vector useful in the treatment of CF as disclosed herein is an AAV3b capsid. AAV3b capsids encompassed for use are described in 2017/106236, and 9,012,224 and 7,892,809, which are incorporated herein in its entirety by reference.

In an embodiment, the AAV capsid can be used for the treatment of CF can be a modified AAV capsid that is derived in whole or in part from the AAV capsid set forth. In some embodiments, the amino acids from an AAV3b capsid can be, or are substituted with amino acids from another capsid of a different AAV serotype, wherein the substituted and/or inserted amino acids can be from any AAV serotype, and can include either naturally occurring or partially or completely synthetic amino acids.

Methods of Treatment

Cystic Fibrosis (CF)

The disease is caused by mutations in the Cystic Fibrosis Transmenbrane Conductance Regulator (CFTR) gene, leading to production of defective CFTR protein, which disrupts chloride transport resulting in markedly impaired water fluxes across various epithelial layers. This leads to ‘sticky’ mucous secretions which obstruct the secretory glands of the lungs, digestive tract and other organs.

Cystic Fibrosis Transmembrane Conductance Regulator (CFTR) Gene.

In some embodiments, the therapeutic transgene is the Cystic Fibrosis Transmembrane Conductance Regulator (CFTR) gene.

As used herein, “cystic fibrosis transmembrane conductance regulator” or “CFTR” refers to a chloride and bicarbonate ion channel that regulates salt and fluid homeostasis. Sequences for CFTR nucleic acids and polypeptides are known for a number of species, including, e.g., human CFTR (NCBI Gene ID: 1080) mRNA (e.g, NCBI Ref Seq: 1.NM_000492.3) and polypeptides (e.g., NP_000483.3). The CFTR glycoprotein has multiple membrane-integrated subunits that form two membrane spanning domains (MSD), two intracellular nucleotide-binding domains (NBD) and a regulatory (R) domain, which acts as a phosphorylation site. MSD1 and MSD2 form the channel pore walls. Opening and closing of the pore is through ATP interactions with cytoplasmic NBD domains, leading to conformational changes of MSD1 and MSD2. Gating and conductance is regulated through R domain phosphorylation with protein kinase A (PKA). The intricate regions of CFTR require processing and maturation to allow precise folding. CFTR structure must satisfy rigorous quality standards to be exported from the endoplasmic reticulum and subsequently transported to the cell surface. CFTR that fails to meet these standards is destined to endoplasmic reticulum-associated protein degradation (ERAD). Such a complex quality control system operates at the detriment of efficiency, decreasing export production of even wild type CFTR to 33% of similar family cell transporters. Cystic fibrosis is a result of mutations that alter CFTR in these domains or the way these domains interact with each other.

The sequence for the CFTR gene product for Homo sapiens is as follows (NP_000483.3):

| (SEQ ID NO: 307) | |
|---|---|
| 1 | mqrsplekas vvsklffswt rpilrkgyrq rlelsdiyqi psvdsadnls eklerewdre |
| 61 | laskknpkli nalrrcffwr fmfygiflyl geytkavqpl llgriiasyd pdnkeersia |
| 121 | iylgiglcll fivrtlllhp aifglhhigm qmriamfsli ykktlklssr vldkisigql |
| 181 | vsllsnnlnk fdeglalahf vwiaplqval lmgliwellq asafcglgfl ivlalfqagl |
| 241 | grmmmkyrdq ragkiserlv itsemieniq svkaycweea mekmienlrq telkltrkaa |
| 301 | yvryfnssaf ffsgffvvfl svlpyalikg iilrkiftti sfcivlrmay trqfpwavqt |
| 361 | wydslgaink iqdflqkqey ktleynittt evymenvtaf weegfgelfe kakqnnnnrk |
| 421 | tsngddslff snfsllgtpv lkdinfkier gqllavagst gagktsllmv imgelepseg |
| 481 | kikhsgrisf csqfswimpg tikeniifgv sydeyryrsv ikacqleedi skfaekdniv |
| 541 | lgeggitlsg gqrarislar avykdadlyl ldspfgyldv ltekeifesc vcklmanktr |
| 601 | ilvtskmehl kkadkililh egssyfygtf selqnlqpdf ssklmgcdsf dqfsaerrns |
| 661 | iltetlhrfs legdapvswt etkkqsfkqt gefgekrkns ilnpinsirk fsivqktplq |
| 721 | mngieedsde plerrlslyp dseqgeailp risvistgpt lqarrrqsvl nlmthsvnqg |
| 781 | qnihrkttas trkvslapqa niteldiysr rlsqetglei seeineedlk ecffddmesi |
| 841 | payttwntyl ryitvhksli fvliwclvif laevaaslyv lwllgntplq dkgnsthsrn |
| 901 | nsyaviitst ssyyvfyiyv gvadtllamg ffrglplyht litvskilhh kmlhsvlqap |
| 961 | mstlntlkag gilnrfskdi ailddllplt ifdfiqllli vigaiavvav lqpyifvatv |
| 1021 | pvivafimlr ayflqtsqql kqlesegrsp ifthlvtslk glwtlrafgr qpyfetlfhk |
| 1081 | alnlhtanwf lylstlrwfq mriemifvif fiavtfisil ttgegegrvg iiltlamnim |
| 1141 | stlqwavnss idvdslmrsv srvfkfidmp tegkptkstk pykngqlsky miienshvkk |
| 1201 | ddiwpsggqm tvkdltakyt eggnaileni sfsispgqry gllgrtgsgk stllsaflrl |
| 1261 | lntegeiqid gvswdsitlq qwrkafgvip qkvfifsgtf rknldpyeqw sdqeiwkvad |
| 1321 | evglrsvieq fpgkldfvly dggcvlshgh kqlmclarsv lskakillld epsahldpvt |
| 1381 | yqiirrtlkq afadctvilc ehrieamlec qqflvieenk vrqydsiqkl lnerslfrqa |
| 1441 | ispsdrvklf phrnsskcks kpqiaalkee teeevqdtrl |

In some embodiments, the therapeutic transgene is a truncated Cystic Fibrosis Transmembrane Conductance Regulator (CFTR) gene including but not limited to N-tail processing mutants of human CFTR (e.g., E60A; A264 or A27-264) (NP_000483.3) as described in e.g. Cebotaru L et al. (2013) J Biol Chem. April 12; 288(15):10505-12. The truncated CFTR mutants described herein can specifically rescue the processing of ΔF508-CFTR, resulting in functional CFTR chloride channels at the cell surface in vitro.

As used herein, mutations in the CFTR gene result in reduced or absent levels of CFTR protein in secretary epithelial cells, primarily in the airways, pancreas and bile duct system of the liver. More than 1900 different mutations in the CFTR gene have been reported. Mutations capable of regulator activity, including, but not limited to, AF508 CFTR and G551D CFTR (see, e.g., http://www.gen-et.sickkids.on.ca/cfni, for CFTR mutations).

TABLE 4
Incidence of 10 most common CFTR mutations

| CFTR Mutation | Allele frequency (%) |
|---|---|
| ΔF508 | 67.9 |
| 394delTT | 7.1 |
| 3659delC | 6.4 |
| S945L | 1.2 |
| R117C | 1.0 |
| R117H | 0.55 |
| T338I | 0.55 |
| G551D | 0.55 |
| R553X | 0.55 |
| I506L | 0.41 |

Impaired function of CFTR reduces the level of chloride ions (CO escaping from the epithelial cells into the overlying mucous layer. Reduced secretion of the ion into the mucus results in a Na⁺:Cl⁻ imbalance which in turn reduces the amount of water absorbed into the mucous layer. As a result, the mucus becomes thick, tacky and resistant to movement by the mucociliary elevator. Retained mucus in the lung becomes a favorable medium for bacterial infection, notably Pseudomonas aeruginosa, fostering repeated pneumonias, lung damage and ultimately lung failure in >95% of patients with CF. Retained mucus in other ductal systems of the pancreas, intestine and the liver biliary system cause obstructions, organ dysfunction and in some cases organ failure.

Gene Editing Molecule

In some embodiments the therapeutic nucleic acid is a gene editing molecule.

Aspects of the technology described herein are outlined here, wherein the rAAV genome comprises, in the 5′ to 3′ direction:

### a 5′ ITR,

a promoter sequence,
an intron sequence,
a therapeutic nucleic acid (e.g. a gene editing molecule)
a poly A sequence, and

### a 3′ ITR.

A therapeutic nucleic acid molecule, as described herein, can be a vector, an expression vector, an inhibitory nucleic acid, an aptamer, a template molecule or cassette (e.g., for gene editing), or a targeting molecule (e.g., for CRISPR-Cas technologies), or any other nucleic acid molecule that one wishes to deliver to a cell. The nucleic acid molecule can be RNA, DNA, or synthetic or modified versions thereof.

In all aspects provided herein, the gene editing nucleic acid sequence encodes a gene editing molecule selected from the group consisting of: a sequence specific nuclease, one or more guide RNA, CRISPR/Cas, a ribonucleoprotein (RNP), or deactivated CAS for CRISPRi or CRISPRa systems, or any combination thereof.

In some embodiments the gene editing molecule is selected from a nuclease, a guide RNA (gRNA), a guide DNA (gDNA), and an activator RNA.

In general, a guide sequence is any polynucleotide sequence having sufficient complementarity with a target polynucleotide sequence to hybridize with the target sequence and direct sequence-specific targeting of an RNA-guided endonuclease complex to the selected genomic target sequence. In some embodiments, a guide RNA binds and e.g., a Cas protein can form a ribonucleoprotein (RNP), for example, a CRISPR/Cas complex.

In some embodiments, the guide RNA (gRNA) sequence comprises a targeting sequence that directs the gRNA sequence to a desired site in the genome, fused to a crRNA and/or tracrRNA sequence that permit association of the guide sequence with the RNA-guided endonuclease. In some embodiments, the degree of complementarity between a guide sequence and its corresponding target sequence, when optimally aligned using a suitable alignment algorithm, is at least 50%, 60%, 75%, 80%, 85%, 90%, 95%, 97.5%, 99%, or more. Optimal alignment can be determined with the use of any suitable algorithm for aligning sequences, such as the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies, ELAND (Illumina, San Diego, Calif.), SOAP, and Maq. In some embodiments, a guide sequence is 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. It is contemplated herein that the targeting sequence of the guide RNA and the target sequence on the target nucleic acid molecule can comprise 1, 2, 3, 4, 5, 6, 7, 8, 9, or 10 mismatches. In some embodiments, the guide RNA sequence comprises a palindromic sequence, for example, the self-targeting sequence comprises a palindrome. The targeting sequence of the guide RNA is typically 19-21 base pairs long and directly precedes the hairpin that binds the entire guide RNA (targeting sequence+hairpin) to a Cas such as Cas9. Where a palindromic sequence is employed as the self-targeting sequence of the guide RNA, the inverted repeat element can be e.g., 9, 10, 11, 12, or more nucleotides in length. Where the targeting sequence of the guide RNA is most often 19-21 bp, a palindromic inverted repeat element of 9 or 10 nucleotides provides a targeting sequence of desirable length. The Cas9-guide RNA hairpin complex can then recognize and cut any nucleotide sequence (DNA or RNA) e.g., a DNA sequence that matches the 19-21 base pair sequence and is followed by a “PAM” sequence e.g., NGG or NGA, or other PAM.

The ability of a guide sequence to direct sequence-specific binding of an RNA-guided endonuclease complex to a target sequence can be assessed by any suitable assay. For example, the components of an RNA-guided endonuclease system sufficient to form an RNA-guided endonuclease complex can be provided to a host cell having the corresponding target sequence, such as by transfection with vectors encoding the components of the RNA-guided endonuclease sequence, followed by an assessment of preferential cleavage within the target sequence, such as by Surveyor assay (Transgenomic™, New Haven, Conn.). Similarly, cleavage of a target polynucleotide sequence can be evaluated in a test tube by providing the target sequence, components of an RNA-guided endonuclease complex, including the guide sequence to be tested and a control guide sequence different from the test guide sequence, and comparing binding or rate of cleavage at the target sequence between the test and control guide sequence reactions. One of ordinary skill in the art will appreciate that other assays can also be used to test gRNA sequences.

A guide sequence can be selected to target any target sequence. In some embodiments, the target sequence is a sequence within a genome of a cell. In some embodiments, the target sequence is the sequence encoding a first guide RNA in a self-cloning plasmid, as described herein. Typically, the target sequence in the genome will include a protospacer adjacent (PAM) sequence for binding of the RNA-guided endonuclease. It will be appreciated by one of skill in the art that the PAM sequence and the RNA-guided endonuclease should be selected from the same (bacterial) species to permit proper association of the endonuclease with the targeting sequence. For example, the PAM sequence for CAS9 is different than the PAM sequence for cpF1. Design is based on the appropriate PAM sequence. To prevent degradation of the guide RNA, the sequence of the guide RNA should not contain the PAM sequence. In some embodiments, the length of the targeting sequence in the guide RNA is 12 nucleotides; in other embodiments, the length of the targeting sequence in the guide RNA is 13, 14, 15, 16, 17, 18, 19, 20, 21, 22, 23, 24, 25, 26, 27, 28, 29, 30, 35 or 40 nucleotides. The guide RNA can be complementary to either strand of the targeted DNA sequence. In some embodiments, when modifying the genome to include an insertion or deletion, the gRNA can be targeted closer to the N-terminus of a protein coding region.

It will be appreciated by one of skill in the art that for the purposes of targeted cleavage by an RNA-guided endonuclease, target sequences that are unique in the genome are preferred over target sequences that occur more than once in the genome. Bioinformatics software can be used to predict and minimize off-target effects of a guide RNA (see e.g., Naito et al. “CRISPRdirect: software for designing CRISPR/Cas guide RNA with reduced off-target sites” Bioinformatics (2014), epub; Heigwer, F., et al. “E-CRISP: fast CRISPR target site identification” Nat. Methods 11, 122-123 (2014); Bae et al. “Cas-OFFinder: a fast and versatile algorithm that searches for potential off-target sites of Cas9 RNA-guided endonucleases” Bioinformatics 30(10):1473-1475 (2014); Aach et al. “CasFinder: Flexible algorithm for identifying specific Cas9 targets in genomes” BioRxiv (2014), among others).

For the S. pyogenes Cas9, a unique target sequence in a genome can include a Cas9 target site of the form MMMMMMMMNNNNNNNNNNNNXGG (SEQ ID NO: 308) where NNNNNNNNNNNNXGG N (SEQ ID NO: 309) is A, G, T, or C; and X can be any nucleotide) has a single occurrence in the genome. A unique target sequence in a genome can include an S. pyogenes Cas9 target site of the form MMMMMMMMMNNNNNNNNNNNXGG (SEQ ID NO: 310) where NNNNNNNNNNNXGG (SEQ ID NO: 311) (N is A, G, T, or C; and X can be any nucleotide) has a single occurrence in the genome. For the S. thermophilus CRISPR1 Cas9, a unique target sequence in a genome can include a Cas9 target site of the form MMMMMMMMNNNNNNNNNNXXAGAAW (SEQ ID NO: 312) where NNNNNNNNNNNNXXAGAAW (SEQ ID NO: 313) (N is A, G, T, or C; X can be any nucleotide; and W is A or T) has a single occurrence in the genome. A unique target sequence in a genome can include an S. thermophilus CRISPR 1 Cas9 target site of the form MMMMMMMMMNNNNNNNNNNXXAGAAW (SEQ ID NO: 314) where NNNNNNNNNNNXXAGAAW (SEQ ID NO: 315) (N is A, G, T, or C; X can be any nucleotide; and W is A or T) has a single occurrence in the genome. For the S. pyogenes Cas9, a unique target sequence in a genome can include a Cas9 target site of the form MMMMMMMMMNNNNNNNNNNXGGXG (SEQ ID NO: 316) where NNNNNNNNNNNNXGGXG (SEQ ID NO: 317) (N is A, G, T, or C; and X can be any nucleotide) has a single occurrence in the genome. A unique target sequence in a genome can include an S. pyogenes Cas9 target site of the form MMMMMMMMMNNNNNNNNNNNXGGXG (SEQ ID NO: 318) where NNNNNNNNNNNXGGXG (SEQ ID NO: 319) (N is A, G, T, or C; and X can be any nucleotide) has a single occurrence in the genome. In each of these sequences “M” may be A, G, T, or C, and need not be considered in identifying a sequence as unique.

In general, a “crRNA/tracrRNA fusion sequence,” as that term is used herein refers to a nucleic acid sequence that is fused to a unique targeting sequence and that functions to permit formation of a complex comprising the guide RNA and the RNA-guided endonuclease. Such sequences can be modeled after CRISPR RNA (crRNA) sequences in prokaryotes, which comprise (i) a variable sequence termed a “protospacer” that corresponds to the target sequence as described herein, and (ii) a CRISPR repeat. Similarly, the tracrRNA (“transactivating CRISPR RNA”) portion of the fusion can be designed to comprise a secondary structure similar to the tracrRNA sequences in prokaryotes (e.g., a hairpin), to permit formation of the endonuclease complex. In some embodiments, the fusion has sufficient complementarity with a tracrRNA sequence to promote one or more of: (1) excision of a guide sequence flanked by tracrRNA sequences in a cell containing the corresponding tracr sequence; and (2) formation of an endonuclease complex at a target sequence, wherein the complex comprises the crRNA sequence hybridized to the tracrRNA sequence. In general, degree of complementarity is with reference to the optimal alignment of the crRNA sequence and tracrRNA sequence, along the length of the shorter of the two sequences. Optimal alignment can be determined by any suitable alignment algorithm, and can further account for secondary structures, such as self-complementarity within either the tracrRNA sequence or crRNA sequence. In some embodiments, the degree of complementarity between the tracrRNA sequence and crRNA sequence along the length of the shorter of the two when optimally aligned is about or more than about 25%, 30%, 40%, 50%, 60%, 70%, 80%, 90%, 95%, 97.5%, 99%, or higher. In some embodiments, the tracrRNA sequence is at least 5, 6, 7, 8, 9, 10, 11, 12, 13, 14, 15, 16, 17, 18, 19, 20, 25, 30, 40, 50, 60, 70, 80, 90, 100, or more nucleotides in length (e.g., 70-80, 70-75, 75-80 nucleotides in length). In one embodiment, the crRNA is less than 60, less than 50, less than 40, less than 30, or less than 20 nucleotides in length. In other embodiments, the crRNA is 30-50 nucleotides in length; in other embodiments the crRNA is 30-50, 35-50, 40-50, 40-45, 45-50 or 50-55 nucleotides in length. In some embodiments, the crRNA sequence and tracrRNA sequence are contained within a single transcript, such that hybridization between the two produces a transcript having a secondary structure, such as a hairpin. In some embodiments, the loop forming sequences for use in hairpin structures are four nucleotides in length, for example, the sequence GAAA. However, longer or shorter loop sequences can be used, as can alternative sequences. The sequences preferably include a nucleotide triplet (for example, AAA), and an additional nucleotide (for example C or G). Examples of loop forming sequences include CAAA and AAAG. In one embodiment, the transcript or transcribed gRNA sequence comprises at least one hairpin. In one embodiment, the transcript or transcribed polynucleotide sequence has at least two or more hairpins. In other embodiments, the transcript has two, three, four or five hairpins. In a further embodiment, the transcript has at most five hairpins. In some embodiments, the single transcript further includes a transcription termination sequence, such as a polyT sequence, for example six T nucleotides. Non-limiting examples of single polynucleotides comprising a guide sequence, a crRNA sequence, and a tracr sequence are as follows (listed 5′ to 3′), where “N” represents a base of a guide sequence, the first block of lower case letters represent the crRNA sequence, and the second block of lower case letters represent the tracr sequence, and the final poly-T sequence represents the transcription terminator: (i) NNNNNNNNNNNNNNNNNNNNgtttttgtactctcaagatttaGAAAtaaatcttgcagaagctacaaagataaggctt catgccgaaatcaacaccctgtcattttatggcagggtgttttcgttatttaaTTTTTT (SEQ ID NO: 320); (ii) NNNNNNNNNNNNNNNNNNNNgtttttgtactctcaGAAAthcagaagctacaaagataaggcttcatgccgaaatca acaccctgtcattttatggcagggtgttttcgttatttaaTTTTTT (SEQ ID NO: 321); (iii) NNNNNNNNNNNNNNNNNNNNgtttttgtactctcaGAAAtgcagaagctacaaagataaggcttcatgccgaaatca acaccctgtcattttatggcagggtgtTTTTTT (SEQ ID NO: 322); (iv) NNNNNNNNNNNNNNNNNNNNgttttagagctaGAAAtagcaagttaaaataaggctagtccgttatcaacttgaaaa agtggcaccgagtcggtgcTTTTTT (SEQ ID NO: 323); (v) NNNNNNNNNNNNNNNNNNNNgttttagagctaGAAATAGcaagttaaaataaggctagtccgttatcaacttgaa aaagtTTTTTTT (SEQ ID NO: 324); and (vi) NNNNNNNNNNNNNNNNNNNNgttttagagctagAAATAGcaagttaaaataaggctagtccgttatcaTTTTTTTTT (SEQ ID NO: 325). In some embodiments, sequences (i) to (iii) are used in combination with Cas9 from S. thermophilus CRISPR1. In some embodiments, sequences (iv) to (vi) are used in combination with Cas9 from S. pyogenes. In some embodiments, the tracrRNA sequence is a separate transcript from a transcript comprising the crRNA sequence.

In some embodiments, a guide RNA can comprise two RNA molecules and is referred to herein as a “dual guide RNA” or “dgRNA.” In some embodiments, the dgRNA may comprise a first RNA molecule comprising a crRNA, and a second RNA molecule comprising a tracrRNA. The first and second RNA molecules may form a RNA duplex via the base pairing between the flagpole on the crRNA and the tracrRNA. When using a dgRNA, the flagpole need not have an upper limit with respect to length.

In other embodiments, a guide RNA can comprise a single RNA molecule and is referred to herein as a “single guide RNA” or “sgRNA.” In some embodiments, the sgRNA can comprise a crRNA covalently linked to a tracrRNA. In some embodiments, the crRNA and tracrRNA can be covalently linked via a linker. In some embodiments, the sgRNA can comprise a stem-loop structure via the base-pairing between the flagpole on the crRNA and the tracrRNA. In some embodiments, a single-guide RNA is at least 50, at least 60, at least 70, at least 80, at least 90, at least 100, at least 110, at least 120 or more nucleotides in length (e.g., 75-120, 75-110, 75-100, 75-90, 75-80, 80-120, 80-110, 80-100, 80-90, 85-120, 85-110, 85-100, 85-90, 90-120, 90-110, 90-100, 100-120, 100-120 nucleotides in length). In some embodiments, a vector or composition thereof comprises a nucleic acid that encodes at least 1 gRNA. For example, the second polynucleotide sequence may encode at least 1 gRNA, at least 2 gRNAs, at least 3 gRNAs, at least 4 gRNAs, at least 5 gRNAs, at least 6 gRNAs, at least 7 gRNAs, at least 8 gRNAs, at least 9 gRNAs, at least 10 gRNAs, at least 11 gRNA, at least 12 gRNAs, at least 13 gRNAs, at least 14 gRNAs, at least 15 gRNAs, at least 16 gRNAs, at least 17 gRNAs, at least 18 gRNAs, at least 19 gRNAs, at least 20 gRNAs, at least 25 gRNA, at least 30 gRNAs, at least 35 gRNAs, at least 40 gRNAs, at least 45 gRNAs, or at least 50 gRNAs. The second polynucleotide sequence may encode between 1 gRNA and 50 gRNAs, between 1 gRNA and 45 gRNAs, between 1 gRNA and 40 gRNAs, between 1 gRNA and 35 gRNAs, between 1 gRNA and 30 gRNAs, between 1 gRNA and 25 different gRNAs, between 1 gRNA and 20 gRNAs, between 1 gRNA and 16 gRNAs, between 1 gRNA and 8 different gRNAs, between 4 different gRNAs and 50 different gRNAs, between 4 different gRNAs and 45 different gRNAs, between 4 different gRNAs and 40 different gRNAs, between 4 different gRNAs and 35 different gRNAs, between 4 different gRNAs and 30 different gRNAs, between 4 different gRNAs and 25 different gRNAs, between 4 different gRNAs and 20 different gRNAs, between 4 different gRNAs and 16 different gRNAs, between 4 different gRNAs and 8 different gRNAs, between 8 different gRNAs and 50 different gRNAs, between 8 different gRNAs and 45 different gRNAs, between 8 different gRNAs and 40 different gRNAs, between 8 different gRNAs and 35 different gRNAs, between 8 different gRNAs and 30 different gRNAs, between 8 different gRNAs and 25 different gRNAs, between 8 different gRNAs and 20 different gRNAs, between 8 different gRNAs and 16 different gRNAs, between 16 different gRNAs and 50 different gRNAs, between 16 different gRNAs and 45 different gRNAs, between 16 different gRNAs and 40 different gRNAs, between 16 different gRNAs and 35 different gRNAs, between 16 different gRNAs and 30 different gRNAs, between 16 different gRNAs and 25 different gRNAs, or between 16 different gRNAs and 20 different gRNAs. Each of the polynucleotide sequences encoding the different gRNAs may be operably linked to a promoter. The promoters that are operably linked to the different gRNAs may be the same promoter. The promoters that are operably linked to the different gRNAs may be different promoters. The promoter may be a constitutive promoter, an inducible promoter, a repressible promoter, or a regulatable promoter.

In some experiments, the guide RNAs will target CFTR sequence targeted regions successful for knock-ins, or knock-out deletions, or for correction of defective genes. Multiple gRNA sequences that bind known CFTR target regions have been designed. Non-limiting examples of gRNA sequences targeting CFTR are listed in Table 3.

In some embodiments the therapeutic nucleic acid is a gene editing molecule targeting CFTR.

In some embodiments the gRNAs target the most common CFTR mutation, a deletion of phenylalanine at position 508 (CFTR F508 del) in exon 11, which causes misfolding, endoplasmic reticulum retention, and early degradation of the CFTR protein.

In some embodiments the gRNAs target CFTR including but not limited to gRNAs targeting CFTR exon 11 or intron 11 together with a donor plasmid encoding wild-type CFTR sequences.

In some embodiments the gRNAs target CFTR mutations including but not limited to gRNAs targeting CFTR exon 11 or intron 11.

In some embodiments the gRNAs target CFTR including but not limited to gRNAs targeting CFTR exon 11 or intron 11 together with a donor plasmid encoding wild-type CFTR sequences.

In some embodiments the gRNAs target a CFTR mutation including but not limited to gRNAs targeting CFTR exon 11 or intron 11 together with a donor plasmid encoding wild-type CFTR sequences.

The gRNA sequences listed in Table 4 uniquely target the CFTR gene within the human genome. These gRNA sequences are for use with WT SpCas9, or as crRNA for use with WT SpCas9 protein, to introduce a DSB for genome editing. These sgRNA sequences were validated in Sanjana N. E., Shalem O., Zhang F. Improved vectors and genome-wide libraries for CRISPR screening. Nat Methods. 2014 August; 11(8):783-4.

TABLE 5
guide RNAs targeting the CFTR gene
(see e.g. https://www.genscript.com/gRNA-
detail/1080/CFTR-CRISPR-guide-RNA.html)

| CFTR CRISPR guide | |
|---|---|
| RNA sequences | gRNA target sequences |
| crRNA1 | CGCTCTATCGCGATTTATCT (SEQ ID NO: |
| | 326) |
| crRNA2 | GAGCGTTCCTCCTTGTTATC (SEQ ID NO: |
| | 327) |
| crRNA3 | TCCAGAAAAAACATCGCCGA (SEQ ID NO: |
| | 328) |
| crRNA4 | GGTATATGTCTGACAATTCC (SEQ ID NO: |
| | 329) |

In some embodiments at least one gene editing molecule is a gRNA or a gDNA.

In some embodiments at least one gene editing molecule is a gRNA for transcription activation with SAM.

In some embodiments at least one gene editing molecule is an activator RNA.

The following gRNA sequences listed in Table 5 uniquely and robustly activate transcription of the endogenous CFTR gene within the human genome when used with the CRISPR/Cas9 Synergistic Activation Mediators (SAM) complex. These gRNA specifically target the first 200 bp upstream of the transcription start site (TSS). These validated sgRNA sequences were published in Konermann S et al. Genome-scale transcriptional activation by an engineered CRISPR-Cas9 complex. Nature, 2015 Jan. 29; 517(7536):583-8.

TABLE 6
gRNA for transcription activation with SAM

| SAM gRNA name | SAM gRNA sequence |
|---|---|
| CFTR SAM guide RNA 1 | CGCTAGAGCAAATTTGGGGC |
| | (SEQ ID NO: 330) |
| CFTR SAM guide RNA 2 | GGGCGGCGAGGGAGCGAAGG |
| | (SEQ ID NO: 331) |
| CFTR SAM guide RNA 3 | TGGCGGGGGTGCGTAGTGGG |
| | (SEQ ID NO: 332) |

In some embodiments the sequence specific nuclease is selected from a nucleic acid-guided nuclease, zinc finger nuclease (ZFN), a meganuclease, a transcription activator-like effector nuclease (TALEN), or a megaTAL.

In some embodiments the sequence specific nuclease is a nucleic acid-guided nuclease selected from a single-base editor, an RNA-guided nuclease, and a DNA-guided nuclease.

The nucleases described herein can be altered, e.g., engineered to design sequence specific nuclease (see e.g., U.S. Pat. No. 8,021,867). Nucleases can be designed using the methods described in e.g., Certo, M T et al. Nature Methods (2012) 9:073-975; U.S. Pat. Nos. 8,304,222; 8,021,867; 8,119,381; 8,124,369; 8,129,134; 8,133,697; 8,143,015; 8,143,016; 8,148,098; or 8,163,514, the contents of each are incorporated herein by reference in their entirety. Alternatively, nuclease with site specific cutting characteristics can be obtained using commercially available technologies e.g., Precision BioSciences' Directed Nuclease Editor™ genome editing technology.

In certain embodiments, the vector construct comprises a homology directed repair template, the guide RNA and/or Cas enzyme, or any other nuclease, are delivered in trans, e.g. by administering i) a nucleic acid encoding a guide RNA, ii) or an mRNA encoding a the desired nuclease, e.g. Cas enzyme, or other nuclease iii) or by administering a ribonucleotide protein (RNP) complex comprising a Cas enzyme and a guide RNA, or iv) e.g., delivery of recombinant nuclease proteins by vector, e.g. viral, plasmid, or another vector.

In some embodiments the nucleic acid-guided nuclease is a CRISPR nuclease.

In one embodiment, a vector can comprise an endonuclease (e.g., Cas9) that is transcriptionally regulated by an inducible promoter. In some embodiments, the endonuclease is on a separate vector, which can be administered to a subject with a vector comprising homology arms and a donor sequence, which can optionally also comprise guide RNA (sgRNAs).

In some embodiments the CRISPR nuclease is a Cas nuclease.

In one embodiment, one can administer a cocktail of vectors. For example a combination different gene editing molecules.

In another embodiment, one can administer gene editing molecules and a second vector containing a therapeutic CFTR gene, such as a truncated CFTR gene.

Immune Barriers

Innate and adaptive immune responses are major obstacles for successful gene transfer. The lung has multilayered, sophisticated defense mechanisms which protect the host from pathogens. Important players in this response include macrophages, dendritic cells, neutrophils, and lymphocytes. Pathogen recognition receptors trigger acute and transient innate immune responses through detection of pathogen-associated molecular patterns. Toll-like receptors, the antiviral cytoplasmic helicases (RIG-I and MDA5), and nucleotide oligomerization domain-like receptors are among the pathogen recognition receptors expressed in the airway epithelium. The recognition of pathogen molecules, as well as some gene transfer vectors, results in the secretion of inflammatory cytokines and maturation of antigen presenting cells.

Physical Barriers

Since the CFTR gene was first cloned in 1989, several gene therapy strategies for correction of CF lung disease have been investigated. However, the delivery of the vector systems has been difficult. This is due, in part, to the multiple, sophisticated pulmonary airway barriers that have evolved to clear or prevent the uptake of foreign particles including but not limited to thick secretions and the secondary effects of chronic infection and inflammation in the CF lung present additional barriers to gene transfer.

The lungs have evolved multiple barriers to prevent foreign particles and pathogens from accessing airway cells. The conducting airway surface is lined by a ciliated epithelium. Cilia are bathed in the periciliary fluid layer. The mucus layer, another important physical barrier, covers the periciliary fluid layer. Mucins, which are secreted by surface airway goblet cells and submucosal glands, are primary components of mucus. The mucus layer traps inhaled particles and removes them by mucociliary clearance. An apical surface glycocalyx, composed of carbohydrate, glycoproteins, and polysaccharides, is another barrier. It binds inhaled particles and prevents them from reaching cell surface receptors.

Described herein is a method for treating cystic fibrosis (CF) comprising administering a viral vector, wherein the viral vector is an Adeno-Associated Virus (AAV) vector containing a therapeutic transgene in a capsid to a subject by bronchial artery catheterization delivery.

The term “modulating” as used herein means increasing or decreasing, e.g. activity, by a measurable amount. Compounds that modulate CFTR activity, by increasing the activity of the CFTR anion channel, are called agonists. Compounds that modulate CFTR activity, by decreasing the activity of the CFTR anion channel, are called antagonists.

The phrase “treating or reducing the severity of an CFTR mediated disease” refers both to treatments for diseases that are directly caused by CFTR activities and alleviation of symptoms of diseases not directly caused by CFTR anion channel activities. Examples of diseases whose symptoms may be affected by CFTR activity include, but are not limited to, Cystic fibrosis, Hereditary emphysema, Hereditary hemo-chromatosis, Coagulation-Fibrinolysis deficiencies, such as Protein C deficiency, Type 1 hereditary angioedema, Lipid processing deficiencies, such as Familial hypercholesterolemia, Type 1 chylomicronemia, Abetalipoproteinemia, Lysosomal storage diseases, such as I-cell disease/Pseudo-Hurler, Mucopolysaccharidoses, Sandhof/Tay-Sachs, Crigler-Najjar type II, Polyendocrinopathy/Hyperinsulemia, Diabetes mellitus, Laron dwarfism, Myleoperoxidase deficiency, Primary hypoparathyroidism, Melanoma, Glycanosis CDG type 1, Hereditary emphysema, Congenital hyperthyroidism, Osteogenesis imperfecta, Hereditary hypofibrinogenemia, ACT deficiency, Diabetes insipidus (DI), Neurophyseal DI, Neprogenic DI, Charcot-Marie Tooth syndrome, Perlizaeus-Merzbacher disease, neurodegenerative diseases such as Alzheimer's disease, Parkinson's disease, Amyotrophic lateral sclerosis, Progressive supranuclear plasy, Pick's disease, several polyglutamine neurological disorders asuch as Huntington, Spinocerebullar ataxia type I, Spinal and bulbar muscular atrophy, Dentatorubal pallidoluysian, and Myotonic dystrophy, as well as Spongiform encephalo-pathies, such as Hereditary Creutzfeldt-Jakob disease, Fabry disease, Straussler-Scheinker syndrome, COPD, dry-eye disease, and Sjogren's disease.

CF Disease-Specific Therapies

The following disease-specific therapies include KALYDECO® (ivacaftor) tablets for oral use. Initial U.S. Approval: 2012 directed to milder (and rarer) mutations that still produce CFTR protein on the epithelial cell surface, ORKAMBI® (lumacaftor/ivacaftor) tablets for oral use. U.S. Approval: 2015 for treatment of CF patients with two copies of the F508del mutation (F508del/F508del) directed to the most common severe mutation, and SYMDEKO™ (tezacaftor/ivacaftor) tablets for oral use. Initial U.S. Approval: 2018 directed to treatment of single F508del heterozygotes and some other mutations not covered by Kalydeco.

Symptomatic Treatments

Sympomatic treatments include nebulized hypertonic saline, dornase alfa and mannitol dry powder to reduce viscosity of airway mucus; antibiotics (often nebulized) to treat endemic Pseudomonas aeruginosa infections; bronchodilators to improve airway patency, steroids, daily chest massage, vibration and pounding to loosen secretions.

Thus there are significant unmet medical need, particularly for the most common, severe mutations.

Intravenous Delivery of the CFTR Gene

Considering the non-airway studies; intravenous vector delivery has been studied in mice but has resulted pre-dominantly in alveolar gene transfer and only low level gene delivery to the epithelia of the bronchial tree.

Delivery of the of the CFTR Gene Via Bronchial Arteries

As described herein, delivery of AAV vectors targeting the systemic arterial route, via the bronchial arteries to the mucous producing bronchial airways will overcome the current limitations of gene therapy vector.

As described herein is a method for treating cystic fibrosis (CF) comprising administering a viral vector, wherein the viral vector is an Adeno-Associated Virus (AAV) vector containing a therapeutic transgene in a capsid to a subject by bronchial artery catheterization delivery.

The bronchial arteries supply arterial blood to the lung and arise most commonly from the descending aorta, although a number of anomalous origins are described. The bronchial arteries run parallel to the airways within the bronchovascular sheath, where small branches supply capillary networks to the structural airways, the mucosa, airway smooth muscle, and adventitia. The largest-diameter bronchial arteries can be seen in the adventitia of the airway. Submucosal capillaries arising from these branches are nearly imperceptible. On the venous side the bronchial capillaries form a complex pattern of anastomoses with the pulmonary venous capillaries and venules, azygous vein and in the proximal airways with a limited complex of bronchial veins. —most, but not all, venous blood flowing to the pulmonary veins and returning to the left atrium.

Of the possible animal models, sheep have lungs closest to human anatomy and physiology and have been extensively used for the study of the bronchial circulation physiology, tolerating vascular studies well in experienced hands. In sheep, the bronchial artery arises as a single large carinal vessel that supplies 80% of the systemic flow to both lungs. The ostial diameter of this artery varies from 1-6 mm and would accept 5 French guiding catheters for vector delivery. The artery descends into the lung supplying blood via branches to the main and minor bronchi as far as the distal terminal bronchioles providing a rich peribronchial capillary plexus of thin vessels (5-20 um in diameter) which lies just below the respiratory epithelium in the sub-mucosa surrounding the mucous secreting glands. At the microscopic level the bronchial artery branches are histologically distinct from their pulmonary arterial counterparts in that they have no clearly defined external elastic lamina. The endothelium of the capillaries arising from these arterioles is of the fenestrated type enhancing the passage of fluid into the bronchial mucosa, as well as the passage of neutrophils across the capillaries via active transport through endothelial cell junctions. These anatomical factors highlight why AAV vectors delivered via the bronchial arteries should have an excellent chance of reaching the sub-mucosal layer of all bronchii and thereby all target cells.

Bronchial Artery Approaches in Humans

As used herein, the term “bronchial artery” refers to arteries that supply the structural elements of the lungs with nutrition and oxygenated blood. The bronchial arterial supply in humans is somewhat variable. There are usually two bronchial arteries that run to the left lung, and one to the right lung. The left bronchial arteries (superior and inferior) arise directly from the thoracic aorta. The single right bronchial artery usually arises from one of the following: 1) the thoracic aorta at a common trunk with the right 3rd posterior intercostal artery 2) the superior bronchial artery on the left side 3) any number of the right intercostal arteries mostly the third right posterior. The bronchial arteries supply blood to the bronchi and connective tissue of the lungs. They travel with and branch with the bronchi, generally ending at the level of the respiratory bronchioles. After supplying nutrients and oxygen to the bronchi and bronchioles the bronchial capillaries anastomose with branches of the pulmonary venules, thereby returning to the pulmonary venous circulation. The bronchial vasculature also supplies the visceral pleura of the lung. Since much of the blood supplied by the bronchial arteries is returned via the pulmonary veins rather than to the right-sided circulation blood returning to the left heart is slightly less oxygenated than blood found at the level of the pulmonary capillary beds.

Bronchial Arterial Catheterization

Bronchial arterial catheterization in humans via a percutaneous approach has been practiced for 33 years, initially for direct chemotherapy treatment for bronchial malignancies and subsequently for the embolisation of patients with severe haemoptysis. Bronchial artery catheterisation is an established technique amongst vascular interventionists. It is regularly performed on cystic fibrosis patients who experience episodes of hemoptysis and would be feasible for therapeutic delivery particularly as their bronchial arteries are considerably dilated (Burke T C. and Mauro M A. (2004) Bronchial artery embolization. Semin Intervent Radiol. 2004 March; 21(1):43-8.)

In one embodiment, the present invention provides a catheter having a drug delivery unit at the distal end thereof to effectively shorten the distance a therapeutic agent must travel through the catheter to reach the target site.

Bronchial Artery System

As used herein, the term “bronchioles” or “bronchiole” refers to passageways by which air passes through the nose or mouth to the alveoli (air sacs) of the lungs, in which branches no longer contain cartilage or glands in their submucosa. They are branches of the bronchi, and are part of the conducting zone of the respiratory system. The bronchioles divide further into smaller terminal bronchioles which are still in the conducting zone and these then divide into the smaller respiratory bronchioles which mark the beginning of the respiratory region.

As described herein, “bronchioles” include terminal and respiratory bronchioles.

The primary bronchi, in each lung, which are the left and right bronchus, give rise to secondary bronchi. These in turn give rise to tertiary bronchi. The tertiary bronchi subdivide into the bronchioles. These are histologically distinct from the tertiary bronchi in that their walls do not have hyaline cartilage and they have club cells in their epithelial lining The epithelium starts as a simple ciliated columnar epithelium and changes to simple ciliated cuboidal epithelium as the bronchioles decreases in size. The diameter of the bronchioles is often said to be less than 1 mm, though this value can range from 5 mm to 0.3 mm. As stated, these bronchioles do not have hyaline cartilage to maintain their patency. Instead, they rely on elastic fibers attached to the surrounding lung tissue for support. The inner lining (lamina propria) of these bronchioles is thin with no glands present, and is surrounded by a layer of smooth muscle. As the bronchioles get smaller they divide into terminal bronchioles. These bronchioles mark the end of the conducting zone, which covers the first division through the sixteenth division of the respiratory tract. Alveoli only become present when the conducting zone changes to the respiratory zone, from the sixteenth through the twenty-third division of the tract.

Terminal Bronchioles

The terminal bronchiole is the most distal segment of the conducting zone. It branches off the lesser bronchioles. Each of the terminal bronchioles divides to form respiratory bronchioles which contain a small number of alveoli. Terminal bronchioles are lined with simple cuboidal epithelium containing club cells. Terminal bronchioles contain a limited number of ciliated cells and no goblet cells. Club cells are non-ciliated, rounded protein-secreting cells. Their secretions are a non-sticky, proteinaceous compound to maintain the airway in the smallest bronchioles. The secretion, called surfactant, reduces surface tension, allowing for bronchioles to expand during inspiration and keeping the bronchioles from collapsing during expiration. Club cells, a stem cell of the respiratory system, produce enzymes that detoxify substances dissolved in the respiratory fluid.

Respiratory Bronchioles

The respiratory bronchioles are the narrowest airways of the lungs, one fiftieth of an inch across. The bronchi divide many times before evolving into the bronchioles. The bronchioles deliver air to the exchange surfaces of the lungs. They are interrupted by alveoli which are thin walled evaginations. Alveolar ducts are distal continuations of the respiratory bronchioles.

Lungs

The lungs are the primary organs of the respiratory system in humans and many other animals including a few fish and some snails. In mammals and most other vertebrates, two lungs are located near the backbone on either side of the heart. Their function in the respiratory system is to extract oxygen from the atmosphere and transfer it into the bloodstream, and to release carbon dioxide from the bloodstream into the atmosphere, in a process of gas exchange. Respiration is driven by different muscular systems in different species. Mammals, reptiles and birds use their different muscles to support and foster breathing. In early tetrapods, air was driven into the lungs by the pharyngeal muscles via buccal pumping, a mechanism still seen in amphibians. In humans, the main muscle of respiration that drives breathing is the diaphragm. The lungs also provide airflow that makes vocal sounds including human speech possible.

The lungs are located in the chest on either side of the heart in the rib cage. They are conical in shape with a narrow rounded apex at the top, and a broad concave base that rests on the convex surface of the diaphragm. The apex of the lung extends into the root of the neck, reaching shortly above the level of the sternal end of the first rib. The lungs stretch from close to the backbone in the rib cage to the front of the chest and downwards from the lower part of the trachea to the diaphragm. The left lung shares space with the heart, and has an indentation in its border called the cardiac notch of the left lung to accommodate this. The front and outer sides of the lungs face the ribs, which make light indentations on their surfaces. The medial surfaces of the lungs face towards the centre of the chest, and lie against the heart, great vessels, and the carina where the trachea divides into the two main bronchi. The cardiac impression is an indentation formed on the surfaces of the lungs where they rest against the heart.

Both lungs have a central recession called the hilum at the root of the lung, where the blood vessels and airways pass into the lungs. There are also bronchopulmonary lymph nodes at the hilum.

The lungs are surrounded by the pulmonary pleurae. The pleurae are two serous membranes; the outer parietal pleura lines the inner wall of the rib cage and the inner visceral pleura directly lines the surface of the lungs. Between the pleurae is a potential space called the pleural cavity containing a thin layer of lubricating pleural fluid. Each lung is divided into lobes by the infoldings of the pleura as fissures. The fissures are double folds of pleura that section the lungs and help in their expansion.

The main or primary bronchi enter the lungs at the hilum and initially branch into secondary bronchi also known as lobar bronchi that supply air to each lobe of the lung. The lobar bronchi branch into tertiary bronchi also known as segmental bronchi and these supply air to the further divisions of the lobes known as bronchopulmonary segments. Each bronchopulmonary segment has its own (segmental) bronchus and arterial supply. Segments for the left and right lung are shown in the table. The segmental anatomy is useful clinically for localising disease processes in the lungs. A segment is a discrete unit that can be surgically removed without seriously affecting surrounding tissue.

The lungs are part of the lower respiratory tract, and accommodate the bronchial airways when they branch from the trachea. The lungs include the bronchial airways that terminate in alveoli, the lung tissue in between, and veins, arteries, nerves and lymphatic vessels. The trachea and bronchi have plexuses of lymph capillaries in their mucosa and submucosa. The smaller bronchi have a single layer and they are absent in the alveoli.

All of the lower respiratory tract including the trachea, bronchi, and bronchioles is lined with respiratory epithelium. This is a ciliated epithelium interspersed with goblet cells which produce mucus, and club cells with actions similar to macrophages. Incomplete rings of cartilage in the trachea and smaller plates of cartilage in the bronchi, keep these airways open. Bronchioles are too narrow to support cartilage and their walls are of smooth muscle, and this is largely absent in the narrower respiratory bronchioles which are mainly just of epithelium. The respiratory tract ends in lobules. Each lobule consists of a respiratory bronchiole, which branches into alveolar ducts and alveolar sacs, which in turn divide into alveoli.

The epithelial cells throughout the respiratory tract secrete epithelial lining fluid (ELF), the composition of which is tightly regulated and determines how well mucociliary clearance works. Alveoli consist of two types of alveolar cell and an alveolar macrophage. The two types of cell are known as type I and type II alveolar cells (also known as pneumocytes). Types I and II make up the walls and alveolar septa. Type I cells provide 95% of the surface area of each alveoli and are flat (“squamous”), and Type II cells generally cluster in the corners of the alveoli and have a cuboidal shape.

Type I are squamous epithelial cells that make up the alveolar wall structure. They have extremely thin walls that enable an easy gas exchange. These type I cells also make up the alveolar septa which separate each alveolus. The septa consist of an epithelial lining and associated basement membranes. Type I cells are not able to divide, and consequently rely on differentiation from Type II cells. Type II are larger and they line the alveoli and produce and secrete epithelial lining fluid, and lung surfactant. Type II cells are able to divide and differentiate to Type I cells.

The alveolar macrophages have an important immunological role. They remove substances which deposit in the alveoli including loose red blood cells that have been forced out from blood vessels. The lung is surrounded by a serous membrane of visceral pleura, which has an underlying layer of loose connective tissue attached to the substance of the lung.

The lower respiratory tract is part of the respiratory system, and consists of the trachea and the structures below this including the lungs. The trachea receives air from the pharynx and travels down to a place where it splits (the carina) into a right and left bronchus. These supply air to the right and left lungs, splitting progressively into the secondary and tertiary bronchi for the lobes of the lungs, and into smaller and smaller bronchioles until they become the respiratory bronchioles. These in turn supply air through alveolar ducts into the alveoli, where the exchange of gases take place. Oxygen breathed in, diffuses through the walls of the alveoli into the enveloping capillaries and into the circulation, and carbon dioxide diffuses from the blood into the lungs to be breathed out.

The bronchi in the conducting zone are reinforced with hyaline cartilage in order to hold open the airways. The bronchioles have no cartilage and are surrounded instead by smooth muscle. Air is warmed to 37° C. (99° F.), humidified and cleansed by the conducting zone; particles from the air being trapped on the mucous layer, then removed by the cilia on the respiratory epithelium lining the passageways.

Pulmonary stretch receptors in the smooth muscle of the airways initiate a reflex known as the Hering-Breuer reflex that prevents the lungs from over-inflation, during forceful inspiration.

Bronchial and Pulmonary Circulation

The lungs have a dual blood supply provided by a bronchial and a pulmonary circulation. The bronchial circulation supplies oxygenated blood to the structural elements and airways of the lungs, through the bronchial arteries that originate from the aorta. There are usually three arteries, two to the left lung and one to the right, and they branch alongside the bronchi and bronchioles. The pulmonary circulation carries deoxygenated blood from the heart to the lungs and returns the oxygenated blood to the heart to supply the rest of the body. The blood volume of the lungs, is about 450 millilitres on average, about 9 percent of the total blood volume of the entire circulatory system. This quantity can easily fluctuate from between one-half and twice the normal volume.

Bronchial Artery

The lungs are served by a dual vascular system: (1) The low pressure pulmonary system (15-30 mmHg) comprises the pulmonary artery arising from the right ventricle carrying de-oxygenated blood (100% of the cardiac output) to the alveoli for gas exchange, then returning oxygenated blood to the left atrium for systemic delivery by the left ventricle. (2) The bronchial arterial system is part of the high pressure left (systemic) circulation (110-140 mmHg) arising from arterial branches on the thoracic aorta. Representing only 0.5% of the cardiac output in normal people, the bronchial arteries are the sole nutrient supply for the airway structures, including the bronchial and bronchiolar epithelium from the trachea to the respiratory bronchioles (1-23 branches of the airway).

The bronchial arteries typically arise from the thoracic aorta at the T3 to T8 levels and also supply the bronchi, vagus nerve, posterior mediastinum, and esophagus. Eighty percent of arteries arise from the T5 to T6 level. There are many bronchial artery anatomic variations described. The more common combinations include a single right intercostobronchial (ICB) trunk with single left bronchial artery, single right ICB truck, and single left bronchial artery arising from a common trunk, and a single right ICB trunk with two left bronchial arteries. Left ICB trunks have not been identified, whereas the right bronchial artery frequently shares origins with an intercostal artery. As many as 20% of bronchial arteries have anomalous origins other than the aorta. Aberrant origins include the subclavian, thyrocervical, internal mammary, innominate, pericardiophrenic, superior intercostals, abdominal aorta, and inferior phrenic arteries. Bronchopulmonary arterial anastomoses can be quite prominent in patients with chronic inflammation or pulmonary hypertension. The pulmonary parenchyma may receive arterial blood supply from transpleural systemic collateral to the bronchial circulation via intercostals, mammary, phrenic, thyro-cervical, axillary, and subclavian arteries.

As described herein, the capillary bed of the bronchial system lies immediately beneath the basement membrane of the pseudo-columnar epithelium of the airways at a distance of ≈5-15 μm, representing the primary source of diffusible nutrients for this cell layer.

An important feature of the bronchial arterial system is that there is no corresponding bronchial vein for return of blood to the heart. Instead, bronchial capillaries, through a complex set of shunting vessels fuse with the small veins of the systemic pulmonary venous system back to the left atrium—some also branch into the azygous vein. This provides the opportunity during a therapeutic delivery to impede flow (and increase vector diffusion) in the bronchial arterial capillary bed by compressing the pulmonary (alveolar) capillaries by over-inflating the anesthetic reservoir bag during the infusion procedure.

Since the airway epithelium is pseudo-columnar, all cells, whether basal epithelial cells, putative progenitor cells, Clara cells (mucus producing), ciliated epithelial cells, or rare cell types such as ionocytes (putative Cl− ion expressing cells) all attach directly to the basement membrane with equal access to the underlying bronchial capillaries.

The turnover rates of the various epithelial cells are poorly understood, particularly in disease states such as CF. Further, it remains unclear which of the cell types provides the bulk of the Cl⁻ ions secreted to the epithelial surface. Recent work suggests that newly discovered ionocytes may be a major source, at least in upper airways.

Animal Models of CF

CF models have been generated in a variety of species (e.g., mice, rats, ferrets, sheep and pigs).

CF Pig Models

Recently, new CF animal models have been developed. Rogers and colleagues generated CFTR-null and CFTR-ΔF508 hetero-zygote pigs and subsequently CFTR-ΔF508 homozygous animals. Advantages of the pig as a CF model include lung anatomy, physiology, histology, and biochemistry that are more similar to humans.

In addition, pigs are more homologous to humans genetically, have a larger body size, and longer life spans. CF pigs manifest several phenotypes present in humans with CF. Loss of CFTR function in pigs results in exocrine pancreatic destruction, pancreatic insufficiency, focal biliary cirrhosis, and micro gallbladder. The penetrance of meconium ileus is 100% in CF pigs. This form of intestinal obstruction is observed in about 15% of newborn humans with CF. CF pig lungs exhibit no inflammation at birth, but interestingly their lung tissue was less frequently sterile compared to wild-type littermates.

When challenged with Staphylococcus aureus intratracheally, CF pigs exhibit reduced bacterial eradication compared to wild-type. The animals spontaneously develop lung disease within the first month after birth characterized by bacterial infection, inflammation, airway injury, and remodeling. The lung disease manifestations are heterogeneous and severity varied from mild to severe.

Ferret Models

Another new CF animal model is the ferret. CFTR^{-/-} ferrets develop meconium ileus with 75% penetrance, pancreatic disease, liver disease, and their lungs are often spontaneously colonized with bacteria including Streptococcus and Staphylococcus species within the first 4 weeks after birth. Progressive development of lung disease, as well as defects in bacterial clearance have also been observed in newborn CF ferrets challenged with bacteria.

Sheep Models

Of the possible animal models, sheep have lungs closest to human anatomy and physiology and have been extensively used for the study of the bronchial circulation physiology, tolerating vascular studies well in experienced hands. Sheep models for CF using CRISPR/Cas9 genome editing and somatic cell nuclear transfer (SCNT) techniques have been generated. CFTR knockout sheep develop severe disease consistent with CF pathology in humans. Of particular relevance were pancreatic fibrosis, intestinal obstruction, and absence of the vas deferens. Also, substantial liver and gallbladder disease may reflect CF liver disease that is evident in humans.

In sheep, the bronchial artery arises as a single large carnal vessel that supplies 80% of the systemic flow to both lungs. The ostial diameter of this artery varies from 1-6 mm and would accept 5 French guiding catheters for vector delivery. The artery descends into the lung supplying blood via branches to the main and minor bronchi up to the distal terminal bronchioles providing a rich peribronchial capillary plexus of thin vessels (which lies just below the respiratory epithelium in the sub-mucosa surrounding the mucous secreting glands). At the microscopic level the bronchial artery branches are histologically distinct from their pulmonary arterial counterparts in that they have no clearly defined external elastic lamina. The endothelium of their capillaries is of the fenestrated type and investigators have demonstrated the passage of fluid into the bronchial mucosa, as well as the passage of neutrophils across the capillaries via active transport through endothelial cell junctions. Sheep may be therefore be a particularly relevant animal to model CF in humans due to the similarities in lung anatomy and development in the two species.

In some embodiments, the population of viral vectors is administered by slow infusion over one to five minutes.

In particular embodiments, repeated catheterizations would for example, need to be spaced at least one week apart with a maximum of ten procedures over one, over two, over three, over four, over five, over ten years. (e.g., at least one, at least two, at least three, at least four, at least five, at least six, at least seven, at least eight, at least nine, at least ten etc., or more administrations) may be employed to achieve the desired level of gene expression over a period of various intervals, e.g., hourly, daily, weekly, monthly, yearly, etc. Dosing can be single dosage or cumulative (serial dosing), and can be readily determined by one skilled in the art. For instance, treatment of a disease or disorder may comprise a one-time administration of an effective dose of a pharmaceutical composition viral vector disclosed herein. Alternatively, treatment of a disease or disorder may comprise multiple administrations of an effective dose of a viral vector carried out over a range of time periods, such as, e.g., once daily, twice daily, trice daily, once every few days, or weekly.

The timing of administration can vary from individual to individual, depending upon such factors as the severity of an individual's symptoms. For example, an effective dose of a viral vector disclosed herein can be administered to an individual once every six months for an indefinite period of time, or until the individual no longer requires therapy. A person of ordinary skill in the art will recognize that the condition of the individual can be monitored throughout the course of treatment and that the effective amount of a virus vector disclosed herein that is administered can be adjusted accordingly.

In some embodiments, the rAAV vectors and/or rAAV genome as disclosed herein can be formulated in a solvent, emulsion or other diluent in an amount sufficient to suspend an rAAV vector disclosed herein. In other aspects of this embodiment, the rAAV vectors and/or rAAV genome as disclosed herein can herein may be formulated in a solvent, emulsion or a diluent in an amount of, e.g., less than about 90% (v/v), less than about 80% (v/v), less than about 70% (v/v), less than about 65% (v/v), less than about 60% (v/v), less than about 55% (v/v), less than about 50% (v/v), less than about 45% (v/v), less than about 40% (v/v), less than about 35% (v/v), less than about 30% (v/v), less than about 25% (v/v), less than about 20% (v/v), less than about 15% (v/v), less than about 10% (v/v), less than about 5% (v/v), or less than about 1% (v/v). In other aspects, the rAAV vectors and/or rAAV genome as disclosed herein can disclosed herein may comprise a solvent, emulsion or other diluent in an amount in a range of, e.g., about 1% (v/v) to 90% (v/v), about 1% (v/v) to 70% (v/v), about 1% (v/v) to 60% (v/v), about 1% (v/v) to 50% (v/v), about 1% (v/v) to 40% (v/v), about 1% (v/v) to 30% (v/v), about 1% (v/v) to 20% (v/v), about 1% (v/v) to 10% (v/v), about 2% (v/v) to 50% (v/v), about 2% (v/v) to 40% (v/v), about 2% (v/v) to 30% (v/v), about 2% (v/v) to 20% (v/v), about 2% (v/v) to 10% (v/v), about 4% (v/v) to 50% (v/v), about 4% (v/v) to 40% (v/v), about 4% (v/v) to 30% (v/v), about 4% (v/v) to 20% (v/v), about 4% (v/v) to 10% (v/v), about 6% (v/v) to 50% (v/v), about 6% (v/v) to 40% (v/v), about 6% (v/v) to 30% (v/v), about 6% (v/v) to 20% (v/v), about 6% (v/v) to 10% (v/v), about 8% (v/v) to 50% (v/v), about 8% (v/v) to 40% (v/v), about 8% (v/v) to 30% (v/v), about 8% (v/v) to 20% (v/v), about 8% (v/v) to 15% (v/v), or about 8% (v/v) to 12% (v/v).

In some embodiment, the rAAV vectors and/or rAAV genome as disclosed herein, of any serotype, including but not limited to encapsulated by any AAV2, AAV9 capsid comprise a therapeutic compound in a therapeutically effective amount. In an embodiment, as used herein, without limitation, the term “effective amount” is synonymous with “therapeutically effective amount”, “effective dose”, or “therapeutically effective dose.” In an embodiment, the effectiveness of a therapeutic compound disclosed herein to treat cystic fibrosis can be determined, without limitation, by observing an improvement in an individual based upon one or more clinical symptoms, and/or physiological indicators associated with CF.

To facilitate delivery of a rAAV vector and/or rAAV genome as disclosed herein, it can be mixed with a carrier or excipient. Carriers and excipients that might be used include saline (especially sterilized, pyrogen-free saline) saline buffers (for example, citrate buffer, phosphate buffer, acetate buffer, and bicarbonate buffer), amino acids, urea, alcohols, ascorbic acid, phospholipids, proteins (for example, serum albumin), EDTA, sodium chloride, liposomes, mannitol, sorbitol, and glycerol. USP grade carriers and excipients are particularly useful for delivery of virions to human subjects.

Pharmaceutical compositions of the present invention comprise an effective amount of one or more modified virus vector(s) (e.g., rAAV vectors) or additional agent(s) dissolved or dispersed in a pharmaceutically acceptable carrier. The phrases “pharmaceutical or pharmacologically acceptable” refer to molecular entities and compositions that do not produce an adverse, allergic or other undesirable reaction, biological effect, when administered to an animal, such as, for example, a human, as appropriate.

The preparation of a pharmaceutical composition that contains at least one modified rAAV vector or additional active ingredient will be known to those of skill in the art in light of the present disclosure, as exemplified by Remington's Pharmaceutical Sciences, 18th Ed., Mack Printing Company, 1990, incorporated herein by reference. Moreover, for animal (e.g., human) administration, it will be understood that preparations should meet sterility, pyrogenicity, general safety and purity standards as required by the U.S. FDA Office of Biological Standards or equivalent governmental regulations in other countries, where applicable.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, and like materials and combinations thereof, as would be known to one of ordinary skill in the art (see, for example, Remington's Pharmaceutical Sciences, 18th Ed., Mack Printing Company, 1990, pp. 1289-1329, incorporated herein by reference). Except insofar as any conventional carrier is incompatible with the active ingredient, its use in the therapeutic or pharmaceutical compositions is contemplated.

The modified vector and/or an agent may be formulated into a pharmaceutical composition in a free base, neutral or salt form. Pharmaceutically acceptable salts include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as sodium, potassium, ammonium, calcium or ferric hydroxides; or such organic bases as isopropylamine, trimethylamine, histidine or procaine.

The practitioner responsible for administration will determine the concentration of active ingredient(s) in a pharmaceutical composition and appropriate dose(s) for the individual subject using routine procedures. In certain embodiments, pharmaceutical compositions may comprise, for example, at least about 0.1% of an active compound (e.g., a modified viral vector, e.g., rAAV vector, a therapeutic agent). In other embodiments, the active compound may comprise between about 2% to about 75% of the weight of the unit, or between about 25% to about 60%, for example, and any range derivable therein.

In one aspect of methods of the present invention a heterologous nucleic acid is delivered to a cell of the vasculature or vascular tissue in vitro for purposes of administering the modified cell to a subject, e.g. through grafting or implantation of tissue. The virus particles may be introduced into the cells at the appropriate multiplicity of infection according to standard transduction methods appropriate. Titers of virus to administer can vary, depending upon the target cell type and number, and the particular virus vector, and can be determined by those of skill in the art without undue experimentation. In one embodiment, 10² infectious units, or at least about 10² infectious units, or at least about 10⁵ infectious units are introduced to a cell.

A “therapeutically effective” amount as used herein is an amount that is sufficient to provide some improvement or benefit to the subject. Alternatively stated, a “therapeutically effective” amount is an amount that will provide some alleviation, mitigation, or decrease in at least one clinical symptom in the subject. Those skilled in the art will appreciate that the therapeutic effects need not be complete or curative, as long as some benefit is provided to the subject. In certain embodiments, the therapeutically effective amount is not curative.

Administration of the virus vectors according to the present invention to a human subject or an animal in need thereof can be by any means known in the art. Preferably, the virus vector is delivered in a therapeutically effective dose in a pharmaceutically acceptable carrier. In one embodiment the vector is administered by way of a stent coated with the modified \ vector, or stent that contains the modified \ vector. A delivery sheath for delivery of vectors to the vasculature is described in U.S. patent application publication 20040193137, which is herein incorporated by reference.

Dosages of the virus vector to be administered to a subject depends upon the mode of administration, the disease or condition to be treated, the individual subject's condition, the particular therapeutic nucleic acid to be delivered, and can be determined in a routine manner. Exemplary doses for achieving therapeutic effects are delivery of virus titers of at least about 10⁵, 10⁶, 10⁷, 10⁸, 10⁹, 10¹⁰, 10¹¹, 10¹², 10¹³, 10¹⁴, 10¹⁵, transducing units or more, and any integer derivable therein, and any range derivable therein. In one embodiment, the dose for administration is about 10⁸-10¹³ transducing units. In one embodiment, the dose for administration is about 10³-10⁸ transducing units.

The dose of modified virions required to achieve a particular therapeutic effect in the units of dose in vector genomes/per kilogram of body weight (vg/kg), will vary based on several factors including, but not limited to: the route of modified virion administration, the level of nucleic acid (encoding untranslated RNA or protein) expression required to achieve a therapeutic effect, the specific disease or disorder being treated, a host immune response to the virion, a host immune response to the expression product, and the stability of the heterologous nucleic acid product. One of skill in the art can readily determine a recombinant virion dose range to treat a patient having a particular disease or disorder based on the aforementioned factors, as well as other factors that are well known in the art.

In particular embodiments, more than one administration (e.g., two, three, four or more administrations) may be employed weekly, monthly, yearly, etc.

Injectables can be prepared in conventional forms, either as liquid solutions or suspensions, solid forms suitable for solution or suspension in liquid prior to injection, or as emulsions. The vector can be delivered locally or systemically. In one embodiment the vector is administered in a depot or sustained-release formulation. Further, the virus vector can be delivered adhered to a surgically implantable matrix (e.g., as described in U.S. Patent Publication No. US-2004-0013645-A1).

The modified parvovirus vectors (e.g AAV vectors or other parvoviruses) disclosed herein may be administered by bronchial artery catherization. See, e.g., U.S. Pat. No. 5,585,362.

In one embodiment, bronchial artery delivery is accompanied by a pulmonary wedge pressure catheterization to determine left atrial pressure.

In one embodiment, the population of viral vectors is administered by slow infusion over one to five minutes.

In one embodiment, pressure is applied to the airway outflow either in periodic intervals or pulsed intervals during infusion.

In one embodiment, pressure is supplied every second to fifth breath for up to 15 seconds.

In one embodiment the pressure is 2-15 mmHg.

In one embodiment the proximity of capillaries carrying the vector to the target site is 5 to 10 microns.

In one embodiment, the modified vector of the invention is administered by a catheter in fluid communication with an inflatable balloon formed from a microporous membrane and delivering through the catheter a solution containing a vector comprising the gene of interest, see for example U.S. patent application publication 2003/0100889, which is herein incorporated by reference in its entirety.

In certain embodiments, in order to increase the effectiveness of the modified recombinant vector of the present invention, it may be desirable to combine the methods of the invention with administration of another agent, or other procedure, effective in the treatment of vascular disease or disorder. For example, in some embodiments, it is contemplated that a conventional therapy or agent including, but not limited to, a pharmacological therapeutic agent, a surgical procedure or a combination thereof, may be combined with vector administration. In a non-limiting example, a therapeutic benefit comprises reduced hypertension in a vascular tissue, or reduced restenosis following vascular or cardiovascular intervention, such as occurs during a medical or surgical procedure.

This process may involve administering the agent(s) and the vector at the same time (e.g., substantially simultaneously) or within a period of time wherein separate administration of the vector and an agent to a cell, tissue or subject produces a desired therapeutic benefit. Administration can be done with a single pharmacological formulation that includes both a modified vector and one or more agents, or by administration to the subject two or more distinct formulations, wherein one formulations includes a vector and the other includes one or more agents. In certain embodiments, the agent is an agent that reduces the immune response, e.g. a TLR-9 inhibitor, cGAS inhibitor, or rapamycin.

Administration of the modified vector may precede, be co-administered with, and/or follow the other agent(s) by intervals ranging from minutes to weeks. In embodiments where the vector and other agent(s) are applied separately to a cell, tissue or subject, one would generally ensure that a significant period of time did not expire between the time of each delivery, such that the vector and agent(s) would still be able to exert an advantageously combined effect on the cell, tissue or subject.

Administration of pharmacological therapeutic agents and methods of administration, dosages, and the like are well known to those of skill in the art (see for example, the “Physicians Desk Reference,” Goodman & Gilman's “The Pharmacological Basis of Therapeutics,” “Remington's Pharmaceutical Sciences,” and “The Merck Index, Eleventh Edition,” incorporated herein by reference in relevant parts), and may be combined with the invention in light of the disclosures herein. Some variation in dosage will necessarily occur depending on the condition of the subject being treated. The person responsible for administration will, in any event, determine the appropriate dose for the individual subject, and such individual determinations are within the skill of those of ordinary skill in the art.

Administration

Dosages of the a viral vector, e.g., rHIV, rAAV vector or rAAV genome as disclosed herein to be administered to a subject depend upon the mode of administration, the disease or condition to be treated and/or prevented, the individual subject's condition, the particular virus vector or capsid, and the nucleic acid to be delivered, and the like, and can be determined in a routine manner. Exemplary doses for achieving therapeutic effects are titers of at least about 10⁵, 10⁶, 10⁷, 10⁸, 10⁹, 10¹⁰, 10¹¹, 10¹², 10¹³, 10¹⁴, 10¹⁵ transducing units, optionally about 10⁸ to about 10¹³ transducing units.

In a further embodiment, administration of viral vector, e.g., rAAV or rHIV vector or rAAV genome as disclosed herein to a subject results in a circulatory half-life of said vector of 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 2 days, 3 days, 4 days, 5 days, 6 days, 7 days, 1 week, 2 weeks, 3 weeks, 4 weeks, one month, two months, three months, four months or more.

In an embodiment, the period of administration of a viral vector, e.g., rAAV vector or rAAV genome as disclosed herein to a subject is an infusion of 1 minute to several hours.

In a further embodiment, gene expression is stopped for a period of time. For example, for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more.

In another embodiment, administration of a viral vector, e.g., rAAV vector or rAAV genome as disclosed herein for the treatment of CF results in an increase in weight by, e.g., at least 0.5 pounds, at least 1 pound, at least 1.5 pounds, at least 2 pounds, at least 2.5 pounds, at least 3 pounds, at least 3.5 pounds, at least 4 pounds, at least 4.5 pounds, at least 5 pounds, at least 5.5 pounds, at least 6 pounds, at least 6.5 pounds, at least 7 pounds, at least 7.5 pounds, at least 8 pounds, at least 8.5 pounds, at least 9 pounds, at least 9.5 pounds, at least 10 pounds, at least 10.5 pounds, at least 11 pounds, at least 11.5 pounds, at least 12 pounds, at least 12.5 pounds, at least 13 pounds, at least 13.5 pounds, at least 14 pounds, at least 14.5 pounds, at least 15 pounds, at least 20 pounds, at least 25 pounds, at least 30 pounds, at least 50 pounds. In another embodiment, an AAV CFTR of any serotype, as disclosed herein for the treatment of CF results in an increase in weight by, e.g., from 0.5 pounds to 50 pounds, from 0.5 pounds to 30 pounds, from 0.5 pounds to 25 pounds, from 0.5 pounds to 20 pounds, from 0.5 pounds to 15 pounds, from 0.5 pounds to ten pounds, from 0.5 pounds to 7.5 pounds, from 0.5 pounds to 5 pounds, from 1 pound to 15 pounds, from 1 pound to 10 pounds, from 1 pound to 7.5 pounds, form 1 pound to 5 pounds, from 2 pounds to ten pounds, from 2 pounds to 7.5 pounds.

Optimized rAAV Vector Genome

In an embodiment, an optimized viral vector, e.g., rAAV vector genome is created from any of the elements disclosed herein and in any combination, including an ITR, a promoter, a secretary peptide, a receptor ligand, a truncated transgene, a microRNA, a poly-A tail, elements capable of increasing or decreasing expression of a heterologous gene, in one embodiment, a therapeutic gene and elements to reduce immunogenicity. Such an optimized viral vector, e.g., rAAV vector genome can be used with any AAV capsid that has tropism for the tissue and cells in which the viral vector, e.g., rAAV vector genome is to be transduced and expressed.

The following non-limiting examples are provided for illustrative purposes only in order to facilitate a more complete understanding of representative embodiments now contemplated. These examples are intended to be a mere subset of all possible contexts in which the viral vectors, e.g., AAV vectors or virions and rAAV vectors may be utilized. Thus, these examples should not be construed to limit any of the embodiments described in the present specification, including those pertaining to AAV virions and rAAV vectors and/or methods and uses thereof. Ultimately, the AAV virions and vectors may be utilized in virtually any context where gene delivery is desired.

It is understood that the foregoing description and the following examples are illustrative only and are not to be taken as limitations upon the scope of the invention. Various changes and modifications to the disclosed embodiments, which will be apparent to those of skill in the art, may be made without departing from the spirit and scope of the present invention. Further, all patents, patent applications, and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents are based on the information available to the applicants and do not constitute any admission as to the correctness of the dates or contents of these documents.

All patents and other publications identified are expressly incorporated herein by reference for the purpose of describing and disclosing, for example, the methodologies described in such publications that could be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

Some embodiments of the technology described herein can be defined according to any of the following numbered paragraphs:
1. A method for treating cystic fibrosis (CF) comprising:
   administering a population of vectors to a plurality of target sites in a subject wherein the vector contains a therapeutic nucleic acid, and wherein the vectors are administered by bronchial artery catheterization delivery comprising,
   placing a catheter into a first bronchial artery and administering a first dose of vector into the catheter to target basal laminar target sites in the family of bronchioles subtended by said bronchial artery,
   and placing the same or different catheter into at least a second bronchial artery to target a second family of bronchioles containing a second population of basal lamina cells.
2. The method of paragraph 1, further comprising placing the same or different catheter into a third bronchial artery to target a third family of bronchioles containing a third population of basal lamina cells, if needed.
3. The method of paragraph 2, further comprising placing the same or different catheter into a fourth bronchial artery to target a fourth family of bronchioles containing a fourth population of basal lamina cells, if needed.
4. The method of paragraph 2, further comprising placing the same or different catheter into a fifth bronchial artery to target a fifth family of bronchioles containing a fifth population of basal lamina cells, if needed.
5. The method of paragraph 1, wherein the first dose is proportional to the first bronchial artery volume (the bronchial vessel blood flow volume including the vessel branches) and the second dose is proportional to the second bronchial artery volume.
6. The method of paragraphs 1-5, wherein a first dose of vector is administered into the catheter to target the first basal lamina target site of a basal/progenitor cell, a club cell, or a ciliated cell in a first set of bronchioles.
7. The method of paragraph 1, wherein the therapeutic nucleic acid is a therapeutic Cystic Fibrosis Transmembrane Conductance Regulator (CFTR) gene.
8. The method of paragraph 1, wherein the therapeutic nucleic acid is a truncated therapeutic Cystic Fibrosis Transmembrane Conductance Regulator (CFTR) gene.
9. The method of paragraph 8, wherein the truncated therapeutic Cystic Fibrosis Transmembrane Conductance Regulator (CFTR) gene is a N-tail processing mutants of CFTR.
10. The method of paragraph 8, wherein the truncated therapeutic Cystic Fibrosis Transmembrane Conductance Regulator (CFTR) gene can specifically rescue the processing of ΔF508-CFTR.
11. The method of paragraph 1, wherein the vector is a DNA or RNA nucleic acid vector.
12. The method of paragraph 1, wherein the vector is a viral vector.
13. The method paragraph 9, wherein the viral vector is selected from any of: an adeno associated virus (AAV), adenovirus, lentivirus vector, or a herpes simplex virus (HSV).
14. The method of paragraph 9, wherein the viral vector is a recombinant AAV (rAAV).
15. The method of paragraph 1, wherein the therapeutic nucleic acid is a gene editing molecule.
16. The method of paragraph 15, wherein the gene editing molecule is selected from a nuclease, a guide RNA (gRNA), a guide DNA (gDNA), and an activator RNA.
17. The gene editing molecule of paragraph 15, wherein at least one gene editing molecule is a gRNA or a gDNA.
18. The method of paragraph 17, wherein the guide RNA is targeting a pathology-causing CFTR mutation.
19. The method of paragraph 18, wherein the guide RNA is selected from Table 4.
20. The gene editing molecule of paragraph 15, wherein the sequence specific nuclease is selected from a nucleic acid-guided nuclease, zinc finger nuclease (ZFN), a meganuclease, a transcription activator-like effector nuclease (TALEN), or a megaTAL.
21. The gene editing molecule of paragraph 15, wherein the sequence specific nuclease is a nucleic acid-guided nuclease selected from a single-base editor, an RNA-guided nuclease, and a DNA-guided nuclease.
22. The gene editing molecule of paragraph 15, wherein at least one gene editing molecule is an activator RNA.
23. The gene editing molecule of paragraph 15, wherein the nucleic acid-guided nuclease is a CRISPR nuclease.
24. The gene editing molecule of paragraph 15, wherein the CRISPR nuclease is a Cas nuclease.
25. The method of paragraphs 1-24, wherein the bronchial artery delivery is accompanied by a pulmonary wedge pressure catheterization and measurement.
26. The method of paragraph 25, wherein the population of viral vectors is administered by slow infusion over one to thirty minutes.
27. The method of paragraph 25, wherein pressure is applied to the respiratory reservoir bag every second to fifth breath for up to fifteen seconds in periodic or pulsed intervals during infusion.
28. The method of paragraph 27, wherein the pressure is supplied every second to fifth breath for up to 15 seconds.
29. The method of paragraph 27, wherein the pressure is 2-15 mmHg.
30. The method of paragraphs 1-29, wherein the proximity to the target site is 5 to 10 microns.
31. The method of paragraphs 1-30, wherein the vector is an AAV capsid containing a nucleic acid sequence containing at least one pair of AAV ITRs flanking a segment encoding CFTK operably linked to a promoter, and wherein at least one capsid protein is selected from the group consisting of VP1, VP2, and VP3 is from the same or different AAV serotype.
32. The method of paragraphs 1-30, further comprising administration of a permeabilization agent.
33. The method of paragraph 31, wherein at least one of the capsid proteins is AAV serotype 9.
34. The method of paragraph 31, wherein all the capsid proteins are AAV serotype 9.
35. The method of paragraph 31, wherein one of the other capsid proteins is from a different serotype.
36. The method of paragraphs 31-34, wherein the AAV ITRs are from different serotypes than at least one capsid protein.
37. The method of paragraphs 31-34, wherein the AAV ITRs are from at least one of the same serotypes as the capsid proteins.

### EXAMPLES

### Example 1: Administering Recombinant AAV9 (rAAV9) Vector Containing the CFTR Gene to CFTR Knockout Pigs by Bronchial Artery Catheterization Delivery

The CF lung is the primary target for gene therapy, as it is the most severely affected organ in CF. As described herein, a CF pig model lacking any CFTR function will be used. The CFTR knockout pig model develops spontaneous lung infections similar to that experienced by human patients with CF.

The bronchial arteries typically arise from the thoracic aorta at the T3 to T8 levels and also supply the bronchi, vagus nerve, posterior mediastinum, and esophagus. Eighty percent of arteries arise from the T5 to T6 level. There are many bronchial artery anatomic variations described. The more common combinations include a single right intercostobronchial (ICB) trunk with single left bronchial artery, or a single right ICB truck, and single left bronchial artery arising from a common trunk, or a single right ICB trunk with two left bronchial arteries. Two bronchial arteries can be seen on either the right or left. Left ICB trunks have not been identified, whereas the right bronchial artery frequently shares origins with an intercostals artery.

As described herein, recombinant AAV9 virus carrying a wildtype CFTR gene copy (rAAV9-wtCFTR) will be delivered to a single segment of a dependent lobe of the lungs of a CFTR knockout pig using bronchial artery catheterization delivery as described in Brinson G M et al. Am J Respir Crit Care Med. (1998) Am J Respir Crit Care Med. 1998 June; 157(6 Pt 1):1951-8. and Burke T C. and Mauro M A. (2004) Semin Intervent Radiol. 2004 March; 21(1):43-8. Additionally, a recombinant AAV9-lacZ virus (rAAV9-lacZ) will be used so that the distribution of gene expression in the whole lung can be evaluated using sensitive and specific histochemical stains.

Recombinant AAV9 Virus Administration and Histochemical Assessment.

The animals will be intubated with a 9 mm cuffed endotracheal tube by oral route. Benzocaine (20%) will be sprayed into the endotracheal tube. An Olympus BF 1T20 flexible fiberoptic bronchoscope will be introduced into the airway. For the bronchial artery catheterization delivery of the rAAV9-wtCFTR a catheter will be inserted from the aorta into a first bronchial artery under fluoroscopic control. A first dose of recombinant AAV9 virus carrying a wildtype CFTR gene copy (rAAV9-wtCFTR) will be administered via the catheter to target the basal lamina cells (basal/progenitor cells, club cells, and ciliated cells etc.) in the first set of bronchioles subtended by the said first bronchial artery. Then the same or different catheter will be introduced into a second bronchial vessel to target a second set of bronchioles with a second dose of viral vectors targeting a second set of basolateral cells (basal/progenitor cells club cells, and ciliated cells). If necessary a third and possibly fourth catheterization will be performed to complete the procedure. The total dose delivered will be divided in proportion to the estimated flow to each bronchial artery based on vessel diameters measured from contrast enhanced fluoroscopic images.

The catheter and scope will be removed and animals will be kept in the supine position for another 10 minutes. The lobes of the CFTR knockout pigs infected with rAAV9-wtCFTR and rAAV9-lacZ by bronchial artery catheterization delivery will be compared weekly for 6 weeks by chest x-ray. Necropsies will be performed at 6 weeks. The lung will be fixed and stained using Xgal staining. Histological sections will show recombinant gene expression primarily in the cells of conducting airways. Biodistribution of the LacZ marker and the response of the airways to the wtCFTR treatment versus the lac-Z vector control will be compared.

### Example 2: Administering Recombinant AAV9 (rAAV9) Vector Containing the CFTR Gene in a Capsid to Wild Type and CFTR Knockout Sheep by Bronchial Artery Catheterization Delivery

The CF lung is the primary target for gene therapy, as it is the most severely affected organ in CF. As described herein, a CF sheepmodel lacking any CFTR function will be used. The CFTR knockout sheep model develops spontaneous lung infections similar to that in human patients with CF.

Sheep generally have a single bronchial artery arising from the aorta at the T2-8 level. The branches of the primary vessel than supply the bronchi, vagus nerve, posterior mediastinum, and esophagus.

As described herein, recombinant AAV9 virus carrying either the wildtype CFTR gene copy (rAAV9-wtCFTR) or the AAV9-lacZ marker will be delivered to individual CFTR knockout sheep or in combination using bronchial artery catheterization delivery as described in Brinson G M et al. Am J Respir Crit Care Med. (1998) Am J Respir Crit Care Med. 1998 June; 157(6 Pt 1):1951-8. and Burke T C. and Mauro M A. (2004) Semin Intervent Radiol. 2004 March; 21(1):43-8.

Recombinant AAV9 virus administration and histochemical assessment.

The animals will be intubated with a 9 mm cuffed endotracheal tube by oral route. Benzocaine (20%) will be sprayed into the endotracheal tube. An Olympus BF 1T20 flexible fiberoptic bronchoscope will be introduced into the airway. For the bronchial artery catheterization delivery of the vector(s) a catheter will be inserted from the aorta into the single bronchial artery. The full dose of recombinant AAV9 virus carrying the wildtype CFTR gene copy (rAAV9-wtCFTR) and/or the lac-Z gene will be administered via the catheter to target the basal lamina target site, (basal/progenitor cell, club cells, and ciliated cells etc.) in the entire population of bronchioles.

The catheter and scope will be removed. The animal will be kept in the supine position for another 10 minutes. The lobes of the CFTR knockout sheep infected with rAAV9-wtCFTR and rAAV9-lacZ by bronchial artery catheterization delivery will be assessed weekly for 6 weeks by chest x-ray.

Necropsies will be performed at 6 weeks. The lung will be fixed and stained using Xgal staining. Histological sections will show recombinant gene expression primarily in alveolar cells conducting airway. Biodistribution of the LacZ marker and the response of the airways to the wtCFTR treatment versus the lac-Z vector control will be compared.

### Example 3: Administering Recombinant AAV9 (rAAV9) Vector Containing the CFTR Gene CF Patients by Bronchial Artery Catheterization Delivery

As described herein is a protocol for human clinical trials for gene therapy using a recombinant AAV9 vector containing an inserted wildtype CFTR gene.

Patient selection. Various criteria will be used in evaluating cystic fibrosis patients for gene therapy using the rAAV9 vectors of the present invention. The following criteria should be generally met by patients undergoing the clinical trials:

(1) Proven diagnosis of cystic fibrosis. Proof will consist of documentation of both, sweat sodium or chloride greater than 60 mEq/I by the pilocarpine iontophoresis method or cystic fibrosis genotype and clinical manifestations of cystic fibrosis.

(2) Gender. Males or females may be used. Only patients who have no chance of procreating during the screening period and six months post AAV treatment will be entered into the study. Over 95% of males with cystic fibrosis have congenital atrophy of the vas deferens and are infertile as a result. Females will be eligible if they are negative on a pregnancy test and use a certified method of birth control during the study.

(3) Severity of disease. To be eligible, a patient must be in adequate clinical condition to safely undergo the planned procedures, i.e. aortic catheterizations/bronchoscopies. An acceptable reserve is defined as having a clinical condition such that the estimated 2-year survival is greater than 50%. Patients will be excluded from clinical trials if they exhibit:

(1) Risk of Complications. Conditions which would place them at increased risk for complications from participating in the study. These conditions include: a) Pneumothorax within the last 12 months; b) Insulin-dependent diabetes; c) Asthma or allergic bronchopulmonary aspergillosis requiring glucocorticoid therapy within the last two months; d) Sputum culture growing a pathogen which does not have in vitro sensitivity to at least two types of antibiotics which could be administered to the patient; e) History of major hemoptysis: Coughing up greater that 250 ml of blood within a 24 hour period during the last year; and f) Any medical condition or laboratory abnormality which, according to the opinion of the investigators, would place the patient at increased risk for complications.

Drug therapy. Patients will be excluded if they have been treated with systemic glucocorticoids within two months prior to initiation of the study.

Inability to comply with protocol. Patients will be excluded if, in the opinion of the investigators, the patient has characteristics which would make compliance with the protocol unlikely, e.g. drug abuse, alcoholism, psychiatric instability, inadequate motivation.

Participation in Other Studies. Patients will be excluded if they have participated in another investigational therapeutic study within the previous 90 days.

Patient evaluation. The following evaluations will be performed at various times throughout the study:

History and physical examination. A history relevant to the manifestations of both cystic fibrosis and unrelated diseases is taken. A full review of systems, medication usage, and drug allergy history is obtained.

Clinical laboratory evaluations: a) Blood: hemoglobin, hematocrit, white blood cell count, white blood cell differential count, platelet count, Westergren sedimentation rate, serum electrolytes (sodium, potassium, chloride, bicarbonate), BUN, creatinine, glucose, uric acid, total protein, albumin, calcium, phosphate, total bilirubin, conjugated bilirubin, AST, ALT, alkaline phosphatase, LDH; b) urine analysis: qualitative protein, blood, glucose, ketones, pH and microscopic examination.

Pulmonary function tests. Testing will meet the standards set by the American Thoracic Society (1987a, 1987b): a) spiromerry using the normal predicted values of Crapo et al. (1981); b) absolute lung volumes (total lung capacity, thoracic gas volume, residual volume); and c) diffusion capacity, single breath. Arterial blood gases and pulse oximetry while breathing room air. (5) Electrocardiogram (12-lead). Postero-anterior and lateral chest X-ray. Thin-cut computerized tomography of the chest. Aerobic bacterial culture of sputum with antibiotic to sensitivities.

Shwachman-Kulczycki score calculation. Sperm count for males. If a sperm count has not been done previously with the results documented, semen analysis will be performed by the Department of Urology,

Bronchoscopy. Patients will be allowed nothing by mouth for 6 hours prior to the procedure. They will be premedicated with 0.2 mg glycopyrrolate and 50 mg meperidine intravenously 30 minutes before broncho-scopy. Electrocardiogram, pulse rate, and pulse oximetry will be continuously monitored. Blood pressure will be monitored every 5 minutes by an automated noninvasive system. Viscous lidocaine 2% (30 ml) will be gargled and expectorated. Lidocaine 4% will be sprayed onto the posterior pharynx and larynx by a hand held atomizer. The bronchoscope will be introduced through the nose in patients without nasal obstruction or evidence of polyps. If the nasal approach cannot be used, the bronchoscope will be introduced orally. 0.05% will be applied topically to the mucosa of one nasal passage with a cotton swab. Lidocaine jelly 2% will be instilled into the same nasal passage. Supplemental oxygen by cannula will be administered at the mouth at 6 liters/minute. Midazolam will be administered intravenously in 1 mg boluses over 15 seconds every 5 minutes until the patient is relaxed but still arousable by verbal stimuli. Additional midazolam will be administered in 1 mg boluses up to every 15 minutes to maintain this level of sedation. A flexible fiberoptic bronchoscope will be introduced transnassally. Lidocaine 2% will be injected through the bronchoscope to anesthetize the larynx and airways as needed.

Bronchoalveolar lavage. 50 ml aliquots of normal saline will be injected through the bronchoscope that has been gently wedged into segmental bronchus. The lavagate will be aspirated into a suction trap. The procedure will be repeated until three aliquots have been administered and recovered.

Bronchial Artery Catherization

Beginning two weeks prior to the bronchial artery catheterization, the patient will start an intensified treatment protocol to reduce respiratory infection and maximize overall condition. For two weeks, the patient will receive two anti-Pseudomonal antibiotics to which their cultured organism is sensitive. Twice a day postural drainage and percussion will be performed. The patient will continue on the remainder of their chronic treatment regimen. This phase will be accomplished either as an inpatient or outpatient. During the subsequent studies, the patient will continue on their previously prescribed medical program. This includes continuation of any oral antibiotics, pancreatic enzymes, theophylline, and vitamin supplements. Aerosolized bronchodilators and antibiotics will also be continued.

A chest X-ray and thin cut CT scan will be used to select an anatomical pulmonary segment that: a) has a degree of disease involvement average for that patient; and b) is in a location such that the patient can be positioned at bronchoscopy so that the segmental bronchus is gravitationally dependent.

For the bronchial artery catheterization delivery of the rAAV9-wtCFTR a catheter will be advanced into the descending aorta from a femoral artery under fluoroscopic control. After identifying the bronchial arterial branching pattern from an aotic angiogram and estimating proportional doses, the catheter will be advanced into the first bronchial vessel and a first dose of recombinant AAV9 virus carrying a wildtype CFTR gene copy (rAAV9-wtCFTR) will be administered to target the first basal lamina target site, (basal/progenitor cells, club cells, and ciliated cells etc.) in the bronchioles subtended by the first bronchial artery. Then, the same or a different catheter will be advanced into a second bronchial vessel to target a second set of bronchioles, followed by a third, fourth or fifth delivery as necessary. The doses delivered to each bronchial artery will be in proportion to the estimated blood flow for each vessel as judged from angiography.

Doses and concentrations of rAAV-wtCFTR will be informed by previous large animal experience in pigs and sheep and previous experience with human CF xenografts Englehardt et al., Nature Genetics 4:27-34 (1993).

Post Bronchial Artery Catherization

Vital signs including blood pressure, pulse, temperature, and respiratory rate will be measured and recorded every five minutes for the first hour, every 15 minutes for the next two hours, every one hour for the next six hours, and every two hours for the next 15 hours, and every four hours for the rest of the week post-transfection. Continuous electrocardiographic and pulse oximetry will be measured for the first 24 hours. The clinical laboratory blood tests that will be listed above, pulse oximetry, and PA and lateral chest X-rays will be performed daily for the first week, twice a week for the second week, and weekly thereafter for six weeks. Thin-cut CT scans will be performed.

Following the administration of the virus, the patients will be kept in an isolation room with full respiratory precautions. The isolation room is a negative pressure room in which the air is filtered and delivered outside. Anyone entering the room will be wearing a gown, mask, eye protection, and gloves. The patient will be in isolation for at least 10 days after initiation of therapy. While in the hospital the patient will have his or her sputum, nasal swab, urine and stool analyzed for shedding of rAAV9-wildtype CFTR recombinant virus using a PCR assay, known in the art.

The following samples and measurements will be obtained during post-transfection bronchoscopies: a) transepithelial electrical potential difference at four sites within the transfected segment and within the segmental bronchus of its mirror image in the opposite lung: b) bronchoalveolar lavage of transfected segment and its mirror image in the opposite lung; c) six cytological brushings of alveolar surface from the transfected segment; and d) six transbronchial biopsies from the transfected segment.

Evaluation of Therapy.

The patient will be carefully monitored for toxicity, immunological response to CFTR protein or adenoviral proteins and efficiency and stability of gene transfer.

### REFERENCES

Brinson G M et al. Am J Respir Crit Care Med. (1998) Am J Respir Crit Care Med. 1998 June; 157(6 Pt 1):1951-8.
Burke T C. and Mauro M A. (2004) Semin Intervent Radiol. 2004 March; 21(1):43-8.
Wilson, J M and Engelhardt, J. U.S. Pat. No. 5,585,362
Oakland M et al. (2012) Mol Ther. 20(6):1108-15.
Cebotaru L et al. (2013) J Biol Chem. April 12; 288(15):10505-12.
Strayer M. et al. (2002) Am J Physiol Lung Cell Mol Physiol 282(3):L394-404.
Venkatesh V C et al. (1995) Am J Physiol. 1995 April; 268(4 Pt 1):L674-82.

1. A method for treating cystic fibrosis (CF) comprising:
administering a population of vectors to a plurality of target sites in a subject wherein the vector contains a therapeutic nucleic acid, and wherein the vectors are administered by bronchial artery catheterization delivery comprising,
placing a catheter into a first bronchial artery and administering a first dose of vector into the catheter to target basal laminar target sites in the family of bronchioles subtended by said bronchial artery,
and placing the same or different catheter into at least a second bronchial artery to target a second family of bronchioles containing a second population of basal lamina cells.

2. The method of claim 1, further comprising placing the same or different catheter into a third bronchial artery to target a third family of bronchioles containing a third population of basal lamina cells; and
if needed further comprising placing the same or different catheter into a fourth bronchial artery to target a fourth family of bronchioles containing a fourth population of basal lamina cells; and
if needed further comprising placing the same or different catheter into a fifth bronchial artery to target a fifth family of bronchioles containing a fifth population of basal lamina cells.

3. (canceled)

4. (canceled)

5. The method of claim 1, wherein the first dose is proportional to the first bronchial artery volume and the second dose is proportional to the second bronchial artery volume.

6. The method of claim 1, wherein a first dose of vector is administered into the catheter to target the first basal lamina target site of a basal/progenitor cell, a club cell, or a ciliated cell in a first set of bronchioles.

7. The method of claim 1, wherein the therapeutic nucleic acid is a therapeutic Cystic Fibrosis Transmembrane Conductance Regulator (CFTR) gene, or is a truncated therapeutic Cystic Fibrosis Transmembrane Conductance Regulator (CFTR) gene, or a gene editing molecule.

8. (canceled)

9. (canceled)

10. The method of claim 7, wherein the truncated therapeutic Cystic Fibrosis Transmembrane Conductance Regulator (CFTR) gene can specifically rescue the processing of ΔF508-CFTR.

11. (canceled)

12. The method of claim 1, wherein the vector is a viral vector.

13. The method claim 12, wherein the viral vector is selected from any of: an adeno-associated virus (AAV), adenovirus, lentivirus vector, or a herpes simplex virus (HSV).

14. (canceled)

15. (canceled)

16. The method of claim 7, wherein the gene editing molecule is selected from a nuclease, a guide RNA (gRNA), a guide DNA (gDNA), and an activator RNA.

17. The method of claim 7, wherein at least one gene editing molecule is a gRNA or a gDNA.

18. The method of claim 17, wherein the guide RNA targets a pathology-causing CFTR mutation and/or is selected from Table 4.

19. (canceled)

20. The method of claim 16, wherein the nuclease is a sequence specific nuclease selected from a nucleic acid-guided nuclease, zinc finger nuclease (ZFN), a meganuclease, a transcription activator-like effector nuclease (TALEN), or a megaTAL, a nucleic acid-guided nuclease selected from a single-base editor, an RNA-guided nuclease, and a DNA-guided nuclease

21. The method of claim 20, wherein the sequence specific nuclease is a nucleic acid-guided nuclease selected from a single-base editor, an RNA-guided nuclease, and a DNA-guided nuclease, and or the nucleic acid-guided nuclease is a CRISPR nuclease.

22. (canceled)

23. (canceled)

24. The method of claim 21, wherein the CRISPR nuclease is a Cas nuclease.

25. The method of claim 1, wherein
a) the bronchial artery delivery is accompanied by a pulmonary wedge pressure catheterization and measurement; and/or
b) the proximity to the target site is 5 to 10 microns.

26. The method of claim 25, wherein:
a) the population of viral vectors is administered by slow infusion over one to thirty minutes; and/or
b) pressure is applied to the respiratory reservoir bag every second to fifth breath for up to fifteen seconds in periodic or pulsed intervals during infusion.

27. (canceled)

28. The method of claim 26, wherein:
a) the pressure is supplied every second to fifth breath for up to 15 seconds; and/or
b) the pressure is 2-15 mmHg.

29. (canceled)

30. (canceled)

31. The method of claim 1, wherein the vector is an AAV particle comprising a capsid encapsidating a nucleic acid sequence containing at least one pair of AAV ITRs flanking a segment encoding CFTK operably linked to a promoter, and wherein the capsid comprises at least one capsid protein selected from the group consisting of VP1, VP2, and VP3, that are each from the same or different AAV serotype.

32. The method of claim 31, wherein the at least one capsid protein is from a serotype selected from the group consisting of AAV serotype 1, AAV serotype 2, AAV serotype 3, AAV serotype 3A, AAV serotype 3B, AAV serotype 4, AAV serotype 5, AAV serotype 6, AAV serotype 7, AAV serotype 8, AAV serotype 9, AAV serotype 10, AAV serotype 11, AAV serotype 12, AAV serotype 13, avian AAV, bovine AAV, canine AAV, equine AAV and/or ovine AAV.

33. (canceled)

34. The method of claim 32, wherein the at least one capsid protein is from AAV serotype 9.

35. (canceled)

36. (canceled)

37. (canceled)

38. (canceled)
